SEQUENCE LISTING (176 sequences)

SEQ ID NO: 1 - protein, 26 aa, synthetic construct
CPKILKKCRRDSDCPGACICRGNGYC

SEQ ID NO: 2 - protein, 26 aa, synthetic construct
CPKILQRCRRDSDCPGACICRGNGYC

SEQ ID NO: 3 - protein, 26 aa, synthetic construct
CPRILKKCRRDSDCPGACICRGNGYC

SEQ ID NO: 4 - protein, 26 aa, synthetic construct
CPKILQRCRRDSDCPGACICLGNGYC

SEQ ID NO: 5 - protein, 26 aa, synthetic construct
CPKILKKCRHDSDCPGACICRGNGYC

SEQ ID NO: 6 - protein, 26 aa, synthetic construct
CFRILKKCRRDSDCPGACICRGNGYC

SEQ ID NO: 7 - protein, 26 aa, synthetic construct
CFRIWKKCRRDSDCPGACICRGNGYC

SEQ ID NO: 8 - protein, 34 aa, synthetic construct
DGGICPRIGRLCRRDSDCPGACICRATRFCGSGY

SEQ ID NO: 9 - protein, 34 aa, synthetic construct
GGICPRIGRLCRRDSDCPGACICRATRFCGSGYD

SEQ ID NO: 10 - protein, 34 aa, synthetic construct
GGICPRIGRLCRRDSDCPGACICRATRFCGSGSD

SEQ ID NO: 11 - protein, 34 aa, synthetic construct
DGGICPRILVYCRRDSDCPGACICIRRTYCGSGS

SEQ ID NO: 12 - protein, 34 aa, synthetic construct
GGICPRILVYCRRDSDCPGACICIRRTYCGSGSD

SEQ ID NO: 13 - protein, 34 aa, synthetic construct
DGGRCPRLLRWCRRDSDCPGACICARGGLCGSGS

SEQ ID NO: 14 - protein, 34 aa, synthetic construct
GGRCPRLLRWCRRDSDCPGACICARGGLCGSGSD

SEQ ID NO: 15 - protein, 34 aa, synthetic construct
DGGVCPRVGWRCRRDSDCPGACICYPTKWCGSGS

SEQ ID NO: 16 - protein, 34 aa, synthetic construct
GGVCPRVGWRCRRDSDCPGACICYPTKWCGSGSD

SEQ ID NO: 17 - protein, 34 aa, synthetic construct
DGGRCCGGYLVCRRDSDCPGACICVFKKHCGSGS

SEQ ID NO: 18 - protein, 34 aa, synthetic construct
GGRCCGGYLVCRRDSDCPGACICVFKKHCGSGSD

SEQ ID NO: 19 - protein, 34 aa, synthetic construct
DGGICPRIGRLCRRDSDCPGACICRGNGYCGSGS

SEQ ID NO: 20 - protein, 34 aa, synthetic construct
GGICPRIGRLCRRDSDCPGACICRGNGYCGSGSD

SEQ ID NO: 21 - protein, 34 aa, synthetic construct
DGGVCPKILKKCRRDSDCPGACICRATRFCGSGS

SEQ ID NO: 22 - protein, 34 aa, synthetic construct
GGVCPKILKKCRRDSDCPGACICRATRFCGSGSD

SEQ ID NO: 23 - protein, 29 aa, synthetic construct
RICPRIGRLCRRDSDCPGACICRATRFCG

SEQ ID NO: 24 - protein, 34 aa, synthetic construct
GGICPRIGRLCKRDSDCPGACICRATRFCGSGSD

SEQ ID NO: 25 - protein, 34 aa, synthetic construct
GGICPRIGRLCRKDSDCPGACICRATRFCGSGSD

SEQ ID NO: 26 - protein, 34 aa, synthetic construct
GGICPRIGRLCRRDSDCPGACICRATRFCGSGKD

SEQ ID NO: 27 - protein, 34 aa, synthetic construct
GGICPRFGRLCRRDSDCPGACICRATRFCGSGSD

SEQ ID NO: 28 - protein, 34 aa, synthetic construct
GGRCPRIGRLCRRDSDCPGACICRATRFCGSGSD

SEQ ID NO: 29 - protein, 29 aa, synthetic construct
  MOD_RES 6: note = 4-fluoro-L-phenylalanine
RVCPRXEKKCRRDSDCPGACICRGNGYCG

SEQ ID NO: 30 - protein, 29 aa, synthetic construct
  MOD_RES 6: note = 4-fluoro-L-phenylalanine
RVCPRXVKKCRRDSDCPGACICRGNGYCG

SEQ ID NO: 31 - protein, 29 aa, synthetic construct
  MOD_RES 6: note = 4-fluoro-L-phenylalanine
RVCPRXWKKCRRDSDCPGACICRGNGYCG

SEQ ID NO: 32 - protein, 29 aa, synthetic construct
  MOD_RES 6: note = 4-fluoro-L-phenylalanine
RVCPRXERKCRRDSDCPGACICRGNGYCG

SEQ ID NO: 33 - protein, 29 aa, synthetic construct
  MOD_RES 6: note = 4-fluoro-L-phenylalanine
RVCPRXVRKCRRDSDCPGACICRGNGYCG

SEQ ID NO: 34 - protein, 29 aa, synthetic construct
  MOD_RES 6: note = 4-fluoro-L-phenylalanine
RVCPRXWRKCRRDSDCPGACICRGNGYCG

SEQ ID NO: 35 - protein, 29 aa, synthetic construct
  MOD_RES 6: note = 4-fluoro-L-phenylalanine
  MOD_RES 7: note = Norleucine
RVCPRXXKACRRDSDCPGACICRGNGYCG

SEQ ID NO: 36 - protein, 34 aa, synthetic construct
  MOD_RES 7: note = 4-fluoro-L-phenylalanine
GGVCPRXEKKCRRDSDCPGACICRGNGYCGSGSD

SEQ ID NO: 37 - protein, 6 aa, synthetic construct
PKILKK

SEQ ID NO: 38 - protein, 6 aa, synthetic construct
PKILQR

SEQ ID NO: 39 - protein, 6 aa, synthetic construct
PRILKK

SEQ ID NO: 40 - protein, 6 aa, synthetic construct
PRILKQ

SEQ ID NO: 41 - protein, 6 aa, synthetic construct
FRILKK

SEQ ID NO: 42 - protein, 6 aa, synthetic construct
FRIWKK

SEQ ID NO: 43 - protein, 34 aa, synthetic construct
DGGVCPKILKKCRRDSDCPGACICRGNGYCGSGS

SEQ ID NO: 44 - protein, 34 aa, synthetic construct
GGVCPKILKKCRRDSDCPGACICRGNGYCGSGSD

SEQ ID NO: 45 - protein, 34 aa, synthetic construct
GGICPRIGRLCQRDSDCPGACICRATRFCGSGSD

SEQ ID NO: 46 - protein, 34 aa, synthetic construct
GGICPRIGRLCRQDSDCPGACICRATRFCGSGSD

SEQ ID NO: 47 - protein, 34 aa, synthetic construct
GGICPRIGRLCRRDSDCPGACICRATQFCGSGSD

SEQ ID NO: 48 - protein, 34 aa, synthetic construct
GGICPRIGRLCQQDSDCPGACICRATRFCGSGSD

SEQ ID NO: 49 - protein, 34 aa, synthetic construct
GGICPRIGRLCQRDSDCPGACICRATQFCGSGSD

SEQ ID NO: 50 - protein, 34 aa, synthetic construct
GGICPRIGRLCRQDSDCPGACICRATQFCGSGSD

SEQ ID NO: 51 - protein, 26 aa, synthetic construct
CPRILKKCRRDSDCPGACVCKGNGYC

SEQ ID NO: 52 - protein, 26 aa, synthetic construct
CPKILQRCRRDSDCPSACICRGNGYC

SEQ ID NO: 53 - protein, 26 aa, synthetic construct
CPRILKKCRRDSDCPGACVCRGNGYC

SEQ ID NO: 54 - protein, 34 aa, synthetic construct
GGICPRIGRLCRRDSDCPGACKCRATRFCGSGSD

SEQ ID NO: 55 - protein, 34 aa, synthetic construct
GGQCFRILKKCRRDSDCPGACICRGNGYCGSGSD

SEQ ID NO: 56 - protein, 34 aa, synthetic construct
GGQCSRILKKCRRDSDCPGACICRGNGYCGSGSD

SEQ ID NO: 57 - protein, 34 aa, synthetic construct
GGQCFRIWKKCRRDSDCPGACICRGNGYCGSGSD

SEQ ID NO: 58 - protein, 34 aa, synthetic construct
KGQCFRIWKKCRRDSDCPGACICRGNGYCGSGSD

SEQ ID NO: 59 - protein, 34 aa, synthetic construct
DGGVCPKILQRCRRDSDCPGACICRGNGYCGSGS

SEQ ID NO: 60 - protein, 26 aa, synthetic construct
CPRILKKCRRDSDCPGECICQGNGYC

SEQ ID NO: 61 - protein, 30 aa, synthetic construct
QRACPRILKKCRRDSDCPGECICQGNGYCG

SEQ ID NO: 62 - protein, 26 aa, synthetic construct
CPRILMPCKVDSDCLPNCTCRPNGFC

SEQ ID NO: 63 - protein, 26 aa, synthetic construct
CPRILMPCKVNDDCLRGCKCLSNGYC

SEQ ID NO: 64 - protein, 26 aa, synthetic construct
CPRILMPCKTDDDCMLDCRCLSNGYC

SEQ ID NO: 65 - protein, 26 aa, synthetic construct
CPRILMKCKTDRDCLTGCTCKRNGYC

SEQ ID NO: 66 - protein, 26 aa, synthetic construct
CPRILMKCKTDRDCLAGCTCKRNGYC

SEQ ID NO: 67 - protein, 26 aa, synthetic construct
CPRILMPCKSDHDCLSGCTCKRNGYC

SEQ ID NO: 68 - protein, 26 aa, synthetic construct
CPRILKKCRRDSDCPGECICKGNGYC

SEQ ID NO: 69 - protein, 26 aa, synthetic construct
CPRILKKCRRDSDCPGECICKGNGYC

SEQ ID NO: 70 - protein, 26 aa, synthetic construct
CPRILKKCRRDSDCPGECICQGNGYC

SEQ ID NO: 71 - protein, 34 aa, synthetic construct; identical to SEQ ID NO: 43
  SITE 1: note = N-terminus is amide bonded to the C-terminus of the C-terminal serine
  SITE 34: note = C-terminus is amide bonded to the N-terminus of the N-terminal aspartic acid

SEQ ID NO: 72 - protein, 34 aa, synthetic construct; identical to SEQ ID NO: 8

SEQ ID NO: 73 - protein, 34 aa, synthetic construct; identical to SEQ ID NO: 8
  SITE 1: note = N-terminus is amide bonded to the C-terminus of the C-terminal tyrosine
  SITE 34: note = C-terminus is amide bonded to the N-terminus of the N-terminal aspartic acid

SEQ ID NO: 74 - protein, 34 aa, synthetic construct; identical to SEQ ID NO: 11

SEQ ID NO: 75 - protein, 34 aa, synthetic construct; identical to SEQ ID NO: 11
  SITE 1: note = N-terminus is amide bonded to the C-terminus of the C-terminal serine
  SITE 34: note = C-terminus is amide bonded to the N-terminus of the N-terminal aspartic acid

SEQ ID NO: 76 - protein, 34 aa, synthetic construct; identical to SEQ ID NO: 13

SEQ ID NO: 77 - protein, 34 aa, synthetic construct; identical to SEQ ID NO: 13
  SITE 1: note = N-terminus is amide bonded to the C-terminus of the C-terminal serine
  SITE 34: note = C-terminus is amide bonded to the N-terminus of the N-terminal aspartic acid

SEQ ID NO: 78 - protein, 34 aa, synthetic construct; identical to SEQ ID NO: 15

SEQ ID NO: 79 - protein, 34 aa, synthetic construct; identical to SEQ ID NO: 15
  SITE 1: note = N-terminus is amide bonded to the C-terminus of the C-terminal serine
  SITE 34: note = C-terminus is amide bonded to the N-terminus of the N-terminal aspartic acid

SEQ ID NO: 80 - protein, 34 aa, synthetic construct; identical to SEQ ID NO: 17

SEQ ID NO: 81 - protein, 34 aa, synthetic construct; identical to SEQ ID NO: 17
  SITE 1: note = N-terminus is amide bonded to the C-terminus of the C-terminal serine
  SITE 34: note = C-terminus is amide bonded to the N-terminus of the N-terminal aspartic acid

SEQ ID NO: 82 - protein, 34 aa, synthetic construct; identical to SEQ ID NO: 19
  SITE 1: note = N-terminus is amide bonded to the C-terminus of the C-terminal serine
  SITE 34: note = C-terminus is amide bonded to the N-terminus of the N-terminal aspartic acid

SEQ ID NO: 83 - protein, 34 aa, synthetic construct; identical to SEQ ID NO: 21
  SITE 1: note = N-terminus is amide bonded to the C-terminus of the C-terminal serine
  SITE 34: note = C-terminus is amide bonded to the N-terminus of the N-terminal aspartic acid

SEQ ID NO: 84 - protein, 29 aa, synthetic construct
RICPRIGRLCRRDSDCPGACICRATRFCG

SEQ ID NO: 85 - protein, 34 aa, synthetic construct; identical to SEQ ID NO: 24
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 86 - protein, 34 aa, synthetic construct; identical to SEQ ID NO: 25
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 87 - protein, 34 aa, synthetic construct; identical to SEQ ID NO: 26
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 88 - protein, 29 aa, synthetic construct
RVCPRILKKCRRDSDCPGACICRGNGYCG

SEQ ID NO: 89 - protein, 29 aa, synthetic construct
  MOD_RES 6: note = 4-fluoro-L-phenylalanine
RVCPRXEKKCRRDSDCPGACICRGNGYCG

SEQ ID NO: 90 - protein, 29 aa, synthetic construct
  MOD_RES 6: note = 4-fluoro-L-phenylalanine
RVCPRXVKKCRRDSDCPGACICRGNGYCG

SEQ ID NO: 91 - protein, 29 aa, synthetic construct
  MOD_RES 6: note = 4-fluoro-L-phenylalanine
RVCPRXWKKCRRDSDCPGACICRGNGYCG

SEQ ID NO: 92 - protein, 29 aa, synthetic construct
  MOD_RES 6: note = 4-fluoro-L-phenylalanine
RVCPRXERKCRRDSDCPGACICRGNGYCG

SEQ ID NO: 93 - protein, 29 aa, synthetic construct
  MOD_RES 6: note = 4-fluoro-L-phenylalanine
RVCPRXVRKCRRDSDCPGACICRGNGYCG

SEQ ID NO: 94 - protein, 29 aa, synthetic construct
  MOD_RES 6: note = 4-fluoro-L-phenylalanine
RVCPRXWRKCRRDSDCPGACICRGNGYCG

SEQ ID NO: 95 - protein, 29 aa, synthetic construct
  MOD_RES 6: note = 4-fluoro-L-phenylalanine
  MOD_RES 7: note = Norleucine
RVCPRXXKACRRDSDCPGACICRGNGYCG

SEQ ID NO: 96 - protein, 34 aa, synthetic construct; identical to SEQ ID NO: 36
  MOD_RES 7: note = 4-fluoro-L-phenylalanine
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 97 - protein, 34 aa, synthetic construct; identical to SEQ ID NO: 27
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 98 - protein, 37 aa, synthetic construct
MDGGICPRIGRLCRRDSDCPGACICRATRFCGSGSGS

SEQ ID NO: 99 - protein, 34 aa, synthetic construct
DGGICPRIGRLCRRDSDCPGACICRATRFCGSGS

SEQ ID NO: 100 - DNA, 80 bp, synthetic construct
  source 1..80: mol_type = other DNA
ctttaagaaggagatatacatatggatggtggcnnktgcnnknnknnknnknnknnktgccgtcgtgattctgactgccc

SEQ ID NO: 101 - DNA, 82 bp, synthetic construct
  source 1..82: mol_type = other DNA
gccccccgtcctagctgcccgaacctgacccgcamnnmnnmnnmnnmnngcaaatgcacgcaccggggcagtcagaatcacg

SEQ ID NO: 102 - DNA, 46 bp, synthetic construct
  source 1..46: mol_type = other DNA
taatacgactcactatagggttaactttaagaaggagatatacata

SEQ ID NO: 103 - DNA, 22 bp, synthetic construct
  source 1..22: mol_type = other DNA
tttccgccccccgtcctagctg

SEQ ID NO: 104 - DNA, 20 bp, synthetic construct
  source 1..20: mol_type = other DNA
  modified_base 17: mod_base = OTHER; note = (PEG18)5
  modified_base 20: mod_base = OTHER; note = Puromycin
ctcccgccccccgtccnccn

SEQ ID NO: 105 - DNA, 78 bp, synthetic construct
  source 1..78: mol_type = other DNA
ctttaagaaggagatatacatatggatggtggcgtatgccccaaaattctgaagaagtgccgtcgtgattctgactgc

SEQ ID NO: 106 - DNA, 87 bp, synthetic construct
  source 1..87: mol_type = other DNA
gccccccgtcctagctgcccgaacctgacccgcaatatccgtttccacggcaaatgcacgcaccggggcagtcagaatcacgacggc

SEQ ID NO: 107 - DNA, 48 bp, synthetic construct
  source 1..48: mol_type = other DNA
taatacgactcactatagggttaactttaagaaggagatatacatatg

SEQ ID NO: 108 - DNA, 63 bp, synthetic construct
  source 1..63: mol_type = other DNA
tttccgccccccgtcctaagaaccagaaccagaacctgcatagtcgggcacgtcgtatgggta

SEQ ID NO: 109 - DNA, 36 bp, synthetic construct
  source 1..36: mol_type = other DNA
tttccgccccccgtcctaagaaccagaaccagaacc

SEQ ID NO: 110 - DNA, 55 bp, synthetic construct
  source 1..55: mol_type = other DNA
cactctttccctacacgacgctcttccgatctaactttaagaaggagatatacat

SEQ ID NO: 111 - DNA, 54 bp, synthetic construct
  source 1..54: mol_type = other DNA
gactggagttcagacgtgtgctcttccgatctagtcgggcacgtcgtatgggta

SEQ ID NO: 112 - DNA, 89 bp, synthetic construct
  source 1..89: mol_type = other DNA
ctttaagaaggagatatacatatggatggtggcatttgcccgcggattgggcggctttgccgtcgtgattctgactgccccggtgcgtg

SEQ ID NO: 113 - DNA, 89 bp, synthetic construct
  source 1..89: mol_type = other DNA
ctttaagaaggagatatacatatgnnkggtggcatttgcccgcggattgggcggctttgccgtcgtgattctgactgccccggtgcgtg

SEQ ID NO: 114 - DNA, 89 bp, synthetic construct
  source 1..89: mol_type = other DNA
ctttaagaaggagatatacatatggatnnkggcatttgcccgcggattgggcggctttgccgtcgtgattctgactgccccggtgcgtg

SEQ ID NO: 115 - DNA, 89 bp, synthetic construct
  source 1..89: mol_type = other DNA
ctttaagaaggagatatacatatggatggtnnkatttgcccgcggattgggcggctttgccgtcgtgattctgactgccccggtgcgtg

SEQ ID NO: 116 - DNA, 89 bp, synthetic construct
  source 1..89: mol_type = other DNA
ctttaagaaggagatatacatatggatggtggcnnktgcccgcggattgggcggctttgccgtcgtgattctgactgccccggtgcgtg

SEQ ID NO: 117 - DNA, 89 bp, synthetic construct
  source 1..89: mol_type = other DNA
ctttaagaaggagatatacatatggatggtggcattnnkccgcggattgggcggctttgccgtcgtgattctgactgccccggtgcgtg

SEQ ID NO: 118 - DNA, 89 bp, synthetic construct
  source 1..89: mol_type = other DNA
ctttaagaaggagatatacatatggatggtggcatttgcnnkcggattgggcggctttgccgtcgtgattctgactgccccggtgcgtg

SEQ ID NO: 119 - DNA, 89 bp, synthetic construct
  source 1..89: mol_type = other DNA
ctttaagaaggagatatacatatggatggtggcatttgcccgnnkattgggcggctttgccgtcgtgattctgactgccccggtgcgtg

SEQ ID NO: 120 - DNA, 89 bp, synthetic construct
  source 1..89: mol_type = other DNA
ctttaagaaggagatatacatatggatggtggcatttgcccgcggnnkgggcggctttgccgtcgtgattctgactgccccggtgcgtg

SEQ ID NO: 121 - DNA, 89 bp, synthetic construct
  source 1..89: mol_type = other DNA
ctttaagaaggagatatacatatggatggtggcatttgcccgcggattnnkcggctttgccgtcgtgattctgactgccccggtgcgtg

SEQ ID NO: 122 - DNA, 89 bp, synthetic construct
  source 1..89: mol_type = other DNA
ctttaagaaggagatatacatatggatggtggcatttgcccgcggattgggnnkctttgccgtcgtgattctgactgccccggtgcgtg

SEQ ID NO: 123 - DNA, 89 bp, synthetic construct
  source 1..89: mol_type = other DNA
ctttaagaaggagatatacatatggatggtggcatttgcccgcggattgggcggnnktgccgtcgtgattctgactgccccggtgcgtg

SEQ ID NO: 124 - DNA, 89 bp, synthetic construct
  source 1..89: mol_type = other DNA
ctttaagaaggagatatacatatggatggtggcatttgcccgcggattgggcggcttnnkcgtcgtgattctgactgccccggtgcgtg

SEQ ID NO: 125 - DNA, 89 bp, synthetic construct
  source 1..89: mol_type = other DNA
ctttaagaaggagatatacatatggatggtggcatttgcccgcggattgggcggctttgcnnkcgtgattctgactgccccggtgcgtg

SEQ ID NO: 126 - DNA, 89 bp, synthetic construct
  source 1..89: mol_type = other DNA
ctttaagaaggagatatacatatggatggtggcatttgcccgcggattgggcggctttgccgtnnkgattctgactgccccggtgcgtg

SEQ ID NO: 127 - DNA, 89 bp, synthetic construct
  source 1..89: mol_type = other DNA
ctttaagaaggagatatacatatggatggtggcatttgcccgcggattgggcggctttgccgtcgtnnktctgactgccccggtgcgtg

SEQ ID NO: 128 - DNA, 89 bp, synthetic construct
  source 1..89: mol_type = other DNA
ctttaagaaggagatatacatatggatggtggcatttgcccgcggattgggcggctttgccgtcgtgatnnkgactgccccggtgcgtg

SEQ ID NO: 129 - DNA, 70 bp, synthetic construct
  source 1..70: mol_type = other DNA
gcacgtcgtatgggtagctgccgctgccgcaaaacctagtagccctgcaaatgcacgcaccggggcagtc

SEQ ID NO: 130 - DNA, 72 bp, synthetic construct
  source 1..72: mol_type = other DNA
ctttaagaaggagatatacatatggatggtggcatttgcccgcggattgggcggctttgccgtcgtgattct

SEQ ID NO: 131 - DNA, 87 bp, synthetic construct
  source 1..87: mol_type = other DNA
gcacgtcgtatgggtagctgccgctgccgcaaaacctagtagccctgcaaatgcacgcaccggggcamnnagaatcacgacggcaaa

SEQ ID NO: 132 - DNA, 87 bp, synthetic construct
  source 1..87: mol_type = other DNA
gcacgtcgtatgggtagctgccgctgccgcaaaacctagtagccctgcaaatgcacgcaccgggmnngtcagaatcacgacggcaaa

SEQ ID NO: 133 - DNA, 87 bp, synthetic construct
  source 1..87: mol_type = other DNA
gcacgtcgtatgggtagctgccgctgccgcaaaacctagtagccctgcaaatgcacgcaccmnngcagtcagaatcacgacggcaaa

SEQ ID NO: 134 - DNA, 87 bp, synthetic construct
  source 1..87: mol_type = other DNA
gcacgtcgtatgggtagctgccgctgccgcaaaacctagtagccctgcaaatgcacgcmnnggggcagtcagaatcacgacggcaaa

SEQ ID NO: 135 - DNA, 87 bp, synthetic construct
  source 1..87: mol_type = other DNA
gcacgtcgtatgggtagctgccgctgccgcaaaacctagtagccctgcaaatgcamnnaccggggcagtcagaatcacgacggcaaa

SEQ ID NO: 136 - DNA, 87 bp, synthetic construct
  source 1..87: mol_type = other DNA
gcacgtcgtatgggtagctgccgctgccgcaaaacctagtagccctgcaaatmnncgcaccggggcagtcagaatcacgacggcaaa

SEQ ID NO: 137 - DNA, 87 bp, synthetic construct
  source 1..87: mol_type = other DNA
gcacgtcgtatgggtagctgccgctgccgcaaaacctagtagccctgcamnngcacgcaccggggcagtcagaatcacgacggcaaa

SEQ ID NO: 138 - DNA, 87 bp, synthetic construct
  source 1..87: mol_type = other DNA
gcacgtcgtatgggtagctgccgctgccgcaaaacctagtagccctmnnaatgcacgcaccggggcagtcagaatcacgacggcaaa

SEQ ID NO: 139 - DNA, 87 bp, synthetic construct
  source 1..87: mol_type = other DNA
gcacgtcgtatgggtagctgccgctgccgcaaaacctagtagcmnngcaaatgcacgcaccggggcagtcagaatcacgacggcaaa

SEQ ID NO: 140 - DNA, 87 bp, synthetic construct
  source 1..87: mol_type = other DNA
gcacgtcgtatgggtagctgccgctgccgcaaaacctagtmnncctgcaaatgcacgcaccggggcagtcagaatcacgacggcaaa

SEQ ID NO: 141 - DNA, 87 bp, synthetic construct
  source 1..87: mol_type = other DNA
gcacgtcgtatgggtagctgccgctgccgcaaaacctmnnagccctgcaaatgcacgcaccggggcagtcagaatcacgacggcaaa

SEQ ID NO: 142 - DNA, 87 bp, synthetic construct
  source 1..87: mol_type = other DNA
gcacgtcgtatgggtagctgccgctgccgcaaaamnnagtagccctgcaaatgcacgcaccggggcagtcagaatcacgacggcaaa

SEQ ID NO: 143 - DNA, 87 bp, synthetic construct
  source 1..87: mol_type = other DNA
gcacgtcgtatgggtagctgccgctgccgcamnncctagtagccctgcaaatgcacgcaccggggcagtcagaatcacgacggcaaa

SEQ ID NO: 144 - DNA, 87 bp, synthetic construct
  source 1..87: mol_type = other DNA
gcacgtcgtatgggtagctgccgctgccmnnaaacctagtagccctgcaaatgcacgcaccggggcagtcagaatcacgacggcaaa

SEQ ID NO: 145 - DNA, 87 bp, synthetic construct
  source 1..87: mol_type = other DNA
gcacgtcgtatgggtagctgccgctmnngcaaaacctagtagccctgcaaatgcacgcaccggggcagtcagaatcacgacggcaaa

SEQ ID NO: 146 - DNA, 87 bp, synthetic construct
  source 1..87: mol_type = other DNA
gcacgtcgtatgggtagctgccmnngccgcaaaacctagtagccctgcaaatgcacgcaccggggcagtcagaatcacgacggcaaa

SEQ ID NO: 147 - DNA, 87 bp, synthetic construct
  source 1..87: mol_type = other DNA
gcacgtcgtatgggtagctmnngctgccgcaaaacctagtagccctgcaaatgcacgcaccggggcagtcagaatcacgacggcaaa

SEQ ID NO: 148 - DNA, 87 bp, synthetic construct
  source 1..87: mol_type = other DNA
gcacgtcgtatgggtamnngccgctgccgcaaaacctagtagccctgcaaatgcacgcaccggggcagtcagaatcacgacggcaaa

SEQ ID NO: 149 - protein, 34 aa, synthetic construct
GGICPRIGRLCRRDSDCPGACICRPTRFCGSGSD

SEQ ID NO: 150 - protein, 34 aa, synthetic construct
GGICPRIGRLCRRDSDCPGACICRKTRFCGSGSD

SEQ ID NO: 151 - protein, 34 aa, synthetic construct
GGICPRIGRLCRRDSDCPGACICRATGFCGSGSD

SEQ ID NO: 152 - protein, 34 aa, synthetic construct
GGICPRIGRLCRRDSDCPGACICRATHFCGSGSD

SEQ ID NO: 153 - protein, 34 aa, synthetic construct
GGICPRIGRLCVRDSDCPGACICRATRFCGSGSD

SEQ ID NO: 154 - protein, 34 aa, synthetic construct
GGICPRIGRLCERDSDCPGACICRATRFCGSGSD

SEQ ID NO: 155 - protein, 34 aa, synthetic construct
GGICPRIGRLCTRDSDCPGACICRATRFCGSGSD

SEQ ID NO: 156 - protein, 34 aa, synthetic construct
GGICPRIGRLCRRDSDCPGACICRATRFCGSGSP

SEQ ID NO: 157 - protein, 34 aa, synthetic construct; identical to SEQ ID NO: 10
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 158 - protein, 34 aa, synthetic construct; identical to SEQ ID NO: 149
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 159 - protein, 34 aa, synthetic construct; identical to SEQ ID NO: 150
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 160 - protein, 34 aa, synthetic construct; identical to SEQ ID NO: 151
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 161 - protein, 34 aa, synthetic construct; identical to SEQ ID NO: 152
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 162 - protein, 34 aa, synthetic construct; identical to SEQ ID NO: 153
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 163 - protein, 34 aa, synthetic construct; identical to SEQ ID NO: 154
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 164 - protein, 34 aa, synthetic construct; identical to SEQ ID NO: 155
  SITE 1: note = N-terminus is amide bonded to the C-terminus of the C-terminal aspartic acid
  SITE 34: note = C-terminus is amide bonded to the N-terminus of the N-terminal glycine

SEQ ID NO: 165 - protein, 34 aa, synthetic construct; identical to SEQ ID NO: 156
  SITE 1: note = N-terminus is amide bonded to the C-terminus of the C-terminal proline
  SITE 34: note = C-terminus is amide bonded to the N-terminus of the N-terminal glycine

SEQ ID NO: 166 - protein, 26 aa, synthetic construct
  MOD_RES 2: note = X is selected from P and modified forms thereof; C and modified forms thereof; and F and modified forms thereof
  MOD_RES 3: note = X is selected from basic amino acid residues including K, R, H and modified forms thereof; and small amino acid residues including S, T, A, G and modified forms thereof
  MOD_RES 4: note = X is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Nle and modified forms thereof
  MOD_RES 5: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and acidic amino acid residues including D, E, hGlu and modified forms thereof
  MOD_RES 6: note = X is selected from small amino acid residues including S, T, A, G and modified forms thereof; aromatic amino acid residues including F, Y, W and modified forms thereof; hydrophobic amino acid residues including V, L, I, M, Nle and modified forms thereof; basic amino acid residues including K, R, H and modified forms thereof; and amide containing amino acid residues including N, Q and modified forms thereof
  MOD_RES 7: note = X is selected from any amino acid residue
  MOD_RES 9: note = X is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof; and hydrophobic amino acid residues including V, L, I, Nle and modified forms thereof
  MOD_RES 10: note = X is selected from basic amino acid residues including K, R, H and modified forms thereof; and amide containing amino acid residues including N, Q and modified forms thereof
  MOD_RES 21: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and basic amino acid residues including K, R, H and modified forms thereof
  MOD_RES 22: note = X is selected from P and modified forms thereof; small amino acid residues including S, T, A, G and modified forms thereof; aromatic amino acid residues including F, Y, W and modified forms thereof; and basic amino acid residues including K, R, H and modified forms thereof
  MOD_RES 23: note = X is selected from amide containing amino acid residues including N, Q and modified forms thereof; small amino acid residues including S, T, A, G and modified forms thereof; and basic amino acid residues including K, R, H and modified forms thereof
  MOD_RES 24: note = X is selected from small amino acid residues including S, T, A, G and modified forms thereof; basic amino acid residues including K, R, H and modified forms thereof; and amide containing amino acid residues including N, Q and modified forms thereof
  MOD_RES 25: note = X is selected from basic amino acid residues including K, R, H and modified forms thereof; aromatic amino acid residues including F, Y, W and modified forms thereof; and hydrophobic amino acid residues including V, L, I, Nle and modified forms thereof
CXXXXXXCXXDSDCPGACICXXXXXC

SEQ ID NO: 167 - protein, 35 aa, synthetic construct
  MOD_RES 1: note = X is absent or is selected from any amino acid residue
  MOD_RES 2: note = X is absent or is selected from any amino acid residue
  MOD_RES 3: note = X is absent or is selected from any amino acid residue
  MOD_RES 4: note = X is absent or is selected from any amino acid residue
  MOD_RES 6: note = X is selected from P and modified forms thereof; C and modified forms thereof; and F and modified forms thereof
  MOD_RES 7: note = X is selected from basic amino acid residues including K, R, H and modified forms thereof; and small amino acid residues including S, T, A, G and modified forms thereof
  MOD_RES 8: note = X is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Nle and modified forms thereof
  MOD_RES 9: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and acidic amino acid residues including D, E, hGlu and modified forms thereof
  MOD_RES 10: note = X is selected from small amino acid residues including S, T, A, G and modified forms thereof; aromatic amino acid residues including F, Y, W and modified forms thereof; hydrophobic amino acid residues including V, L, I, M, Nle and modified forms thereof; basic amino acid residues including K, R, H and modified forms thereof; and amide containing amino acid residues including N, Q and modified forms thereof
  MOD_RES 11: note = X is selected from any amino acid residue
  MOD_RES 13: note = X is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof (e.g. E); and hydrophobic amino acid residues including V, L, I, Nle and modified forms thereof (e.g. V)
  MOD_RES 14: note = X is selected from basic amino acid residues including K, R, H and modified forms thereof; and amide containing amino acid residues including N, Q and modified forms thereof
  MOD_RES 25: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and basic amino acid residues including K, R, H and modified forms thereof
  MOD_RES 26: note = X is selected from P and modified forms thereof; small amino acid residues including S, T, A, G and modified forms thereof; aromatic amino acid residues including F, Y, W and modified forms thereof; and basic amino acid residues including K, R, H and modified forms thereof
  MOD_RES 27: note = X is selected from amide containing amino acid residues including N, Q and modified forms thereof; small amino acid residues including S, T, A, G and modified forms thereof; and basic amino acid residues including K, R, H and modified forms thereof
  MOD_RES 28: note = X is selected from small amino acid residues including S, T, A, G and modified forms thereof; basic amino acid residues including K, R, H and modified forms thereof; and amide containing amino acid residues including N, Q and modified forms thereof
  MOD_RES 29: note = X is selected from basic amino acid residues including K, R, H and modified forms thereof; aromatic amino acid residues including F, Y, W and modified forms thereof; and hydrophobic amino acid residues including V, L, I, Nle and modified forms thereof
  MOD_RES 31: note = X is absent or is selected from any amino acid residue
  MOD_RES 32: note = X is absent or is selected from any amino acid residue
  MOD_RES 33: note = X is absent or is selected from any amino acid residue
  MOD_RES 34: note = X is absent or is selected from any amino acid residue
  MOD_RES 35: note = X is absent or is selected from any amino acid residue
XXXXCXXXXXXCXXDSDCPGACICXXXXXCXXXXX

SEQ ID NO: 168 - protein, 29 aa, synthetic construct
  MOD_RES 1: note = X is absent or is selected from any amino acid residue
  MOD_RES 2: note = X is absent or is selected from any amino acid residue
  MOD_RES 4: note = X is selected from P and modified forms thereof; C and modified forms thereof; and F and modified forms thereof
  MOD_RES 5: note = X is selected from basic amino acid residues including K, R, H and modified forms thereof; and small amino acid residues including S, T, A, G and modified forms thereof
  MOD_RES 6: note = X is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Nle and modified forms thereof
  MOD_RES 7: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and acidic amino acid residues including D, E, hGlu and modified forms thereof
  MOD_RES 8: note = X is selected from small amino acid residues including S, T, A, G and modified forms thereof; aromatic amino acid residues including F, Y, W and modified forms thereof; hydrophobic amino acid residues including V, L, I, M, Nle and modified forms thereof; basic amino acid residues including K, R, H and modified forms thereof; and amide containing amino acid residues including N, Q and modified forms thereof
  MOD_RES 9: note = X is selected from any amino acid residue
  MOD_RES 11: note = X is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof (e.g. E); and hydrophobic amino acid residues including V, L, I, Nle and modified forms thereof (e.g. V)
  MOD_RES 12: note = X is selected from basic amino acid residues including K, R, H and modified forms thereof; and amide containing amino acid residues including N, Q and modified forms thereof
  MOD_RES 23: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and basic amino acid residues including K, R, H and modified forms thereof
  MOD_RES 24: note = X is selected from P and modified forms thereof; small amino acid residues including S, T, A, G and modified forms thereof; aromatic amino acid residues including F, Y, W and modified forms thereof; and basic amino acid residues including K, R, H and modified forms thereof
  MOD_RES 25: note = X is selected from amide containing amino acid residues including N, Q and modified forms thereof; small amino acid residues including S, T, A, G and modified forms thereof; and basic amino acid residues including K, R, H and modified forms thereof
  MOD_RES 26: note = X is selected from small amino acid residues including S, T, A, G and modified forms thereof; basic amino acid residues including K, R, H and modified forms thereof; and amide containing amino acid residues including N, Q and modified forms thereof
  MOD_RES 27: note = X is selected from basic amino acid residues including K, R, H and modified forms thereof; aromatic amino acid residues including F, Y, W and modified forms thereof; and hydrophobic amino acid residues including V, L, I, Nle and modified forms thereof
  MOD_RES 29: note = X is absent or is selected from any amino acid residue
XXCXXXXXXCXXDSDCPGACICXXXXXCX

SEQ ID NO: 169 - protein, 28 aa, synthetic construct
  MOD_RES 1: note = X is absent or is selected from at least one of a proteinaceous moiety consisting of from about 1 to about 50 amino acid residues (and all integer residues in between), and a protecting moiety
  MOD_RES 3: note = X is selected from P and modified forms thereof; C and modified forms thereof; and F and modified forms thereof
  MOD_RES 4: note = X is selected from basic amino acid residues including K, R, H and modified forms thereof; and small amino acid residues including S, T, A, G and modified forms thereof
  MOD_RES 5: note = X is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Nle and modified forms thereof
  MOD_RES 6: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and acidic amino acid residues including D, E, hGlu and modified forms thereof
  MOD_RES 7: note = X is selected from small amino acid residues including S, T, A, G and modified forms thereof; aromatic amino acid residues including F, Y, W and modified forms thereof; hydrophobic amino acid residues including V, L, I, M, Nle and modified forms thereof; basic amino acid residues including K, R, H and modified forms thereof; and amide containing amino acid residues including N, Q and modified forms thereof
  MOD_RES 8: note = X is selected from any amino acid residue
  MOD_RES 10: note = X is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof (e.g. E); and hydrophobic amino acid residues including V, L, I, Nle and modified forms thereof (e.g. V)
  MOD_RES 11: note = X is selected from basic amino acid residues including K, R, H and modified forms thereof; and amide containing amino acid residues including N, Q and modified forms thereof
  MOD_RES 22: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and basic amino acid residues including K, R, H and modified forms thereof
  MOD_RES 23: note = X is selected from P and modified forms thereof; small amino acid residues including S, T, A, G and modified forms thereof; aromatic amino acid residues including F, Y, W and modified forms thereof; and basic amino acid residues including K, R, H and modified forms thereof
  MOD_RES 24: note = X is selected from amide containing amino acid residues including N, Q and modified forms thereof; small amino acid residues including S, T, A, G and modified forms thereof; and basic amino acid residues including K, R, H and modified forms thereof
  MOD_RES 25: note = X is selected from small amino acid residues including S, T, A, G and modified forms thereof; basic amino acid residues including K, R, H and modified forms thereof; and amide containing amino acid residues including N, Q and modified forms thereof
  MOD_RES 26: note = X is selected from basic amino acid residues including K, R, H and modified forms thereof; aromatic amino acid residues including F, Y, W and modified forms thereof; and hydrophobic amino acid residues including V, L, I, Nle and modified forms thereof
  MOD_RES 28: note = X is absent or is selected from at least one of a proteinaceous moiety consisting of from about 1 to about 50 amino acid residues (and all integer residues in between), and a protecting moiety
XCXXXXXXCXXDSDCPGACICXXXXXCX

SEQ ID NO: 170 - protein, 26 aa, synthetic construct
  MOD_RES 2: note = X is selected from P and modified forms thereof; C and modified forms thereof; and F and modified forms thereo
  MOD_RES 3: note = X is selected from basic amino acid residues including K, R, H and modified forms thereof; and small amino acid residues including S, T, A, G and modified forms thereof
  MOD_RES 4: note = X is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Nle and modified forms thereof
  MOD_RES 5: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and acidic amino acid residues including D, E, hGlu and modified forms thereof
  MOD_RES 6: note = X is selected from small amino acid residues including S, T, A, G and modified forms thereof; aromatic amino acid residues including F, Y, W and modified forms thereof; hydrophobic amino acid residues including V, L, I, M, Nle and modified forms thereof; basic amino acid residues including K, R, H and modified forms thereof; and amide containing amino acid residues including N, Q and modified forms thereof
  MOD_RES 7: note = X is selected from any amino acid residue
  MOD_RES 9: note = X is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof (e.g. E); and hydrophobic amino acid residues including V, L, I, Nle and modified forms thereof (e.g. V)
  MOD_RES 10: note = X is selected from basic amino acid residues including K, R, H and modified forms thereof; and amide containing amino acid residues including N, Q and modified forms thereof
  MOD_RES 15: note = X is selected from P and modified forms thereof; and hydrophobic amino acid residues including V, L, I, M, Nle and modified forms thereof
  MOD_RES 16: note = X is selected from small amino acid residues including S, T, A, G and modified forms thereof
  MOD_RES 17: note = X is selected from small amino acid residues including S, T, A, G and modified forms thereof; basic amino acid residues including K, R, H and modified forms thereof; amide containing amino acid residues including N, Q and modified forms thereof; and acidic amino acid residues including D, E, hGlu and modified forms thereof
  MOD_RES 19: note = X is selected from hydrophobic amino acid residues including V, L, I, M, Nle and modified forms thereof; small amino acid residues including S, T, A, G and modified forms thereof; and basic amino acid residues including K, R, H and modified forms thereof
  MOD_RES 21: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and basic amino acid residues including K, R, H and modified forms thereof
  MOD_RES 22: note = X is selected from P and modified forms thereof; small amino acid residues including S, T, A, G and modified forms thereof; aromatic amino acid residues including F, Y, W and modified forms thereof; and basic amino acid residues including K, R, H and modified forms thereof
  MOD_RES 23: note = X is selected from amide containing amino acid residues including N, Q and modified forms thereof; small amino acid residues including S, T, A, G and modified forms thereof; and basic amino acid residues including K, R, H and modified forms thereof
  MOD_RES 24: note = X is selected from small amino acid residues including S, T, A, G and modified forms thereof; basic amino acid residues including K, R, H and modified forms thereof; and amide containing amino acid residues including N, Q and modified forms thereof
  MOD_RES 25: note = X is selected from basic amino acid residues including K, R, H and modified forms thereof; aromatic amino acid residues including F, Y, W and modified forms thereof; and hydrophobic amino acid residues including V, L, I, Nle and modified forms thereof
CXXXXXXCXXDSDCXXXCXCXXXXXC

SEQ ID NO: 171 - protein, 35 aa, synthetic construct
  MOD_RES 1: note = X is absent or is selected from any amino acid residue
  MOD_RES 2: note = X is absent or is selected from any amino acid residue
  MOD_RES 3: note = X is absent or is selected from any amino acid residue
  MOD_RES 4: note = X is absent or is selected from any amino acid residue
  MOD_RES 6: note = X is selected from P and modified forms thereof; C and modified forms thereof; and F and modified forms thereof
  MOD_RES 7: note = X is selected from basic amino acid residues including K, R, H and modified forms thereof; and small amino acid residues including S, T, A, G and modified forms thereof
  MOD_RES 8: note = X is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Nle and modified forms thereof
  MOD_RES 9: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and acidic amino acid residues including D, E, hGlu and modified forms thereof
  MOD_RES 10: note = X is selected from small amino acid residues including S, T, A, G and modified forms thereof; aromatic amino acid residues including F, Y, W and modified forms thereof; hydrophobic amino acid residues including V, L, I, M, Nle and modified forms thereof; basic amino acid residues including K, R, H and modified forms thereof; and amide containing amino acid residues including N, Q and modified forms thereof
  MOD_RES 11: note = X is selected from any amino acid residue
  MOD_RES 13: note = X is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof (e.g. E); and hydrophobic amino acid residues including V, L, I, Nle and modified forms thereof (e.g. V)
  MOD_RES 14: note = X is selected from basic amino acid residues including K, R, H and modified forms thereof; and amide containing amino acid residues including N, Q and modified forms thereof
  MOD_RES 19: note = X is selected from P and modified forms thereof; and hydrophobic amino acid residues including V, L, I, M, Nle and modified forms thereof
  MOD_RES 20: note = X is selected from small amino acid residues including S, T, A, G and modified forms thereof
  MOD_RES 21: note = X is selected from small amino acid residues including S, T, A, G and modified forms thereof; basic amino acid residues including K, R, H and modified forms thereof; amide containing amino acid residues including N, Q and modified forms thereof; and acidic amino acid residues including D, E, hGlu and modified forms thereof
  MOD_RES 23: note = X is selected from hydrophobic amino acid residues including V, L, I, M, Nle and modified forms thereof; small amino acid residues including S, T, A, G and modified forms thereof; and basic amino acid residues including K, R, H and modified forms thereof
  MOD_RES 25: note = X is selected from small amino acid residues including S, T, A, G and modified forms thereof; aromatic amino acid residues including F, Y, W and modified forms thereof; hydrophobic amino acid residues including V, L, I, Nle and modified forms thereof; and basic amino acid residues including K, R, H and modified forms thereof
  MOD_RES 26: note = X is selected from P and modified forms thereof; small amino acid residues including S, T, A, G and modified forms thereof; aromatic amino acid residues including F, Y, W and modified forms thereof; and basic amino acid residues including K, R, H and modified forms thereof
  MOD_RES 27: note = X is selected from amide containing amino acid residues including N, Q and modified forms thereof; small amino acid residues including S, T, A, G and modified forms thereof; and basic amino acid residues including K, R, H and modified forms thereof
  MOD_RES 28: note = X is selected from small amino acid residues including S, T, A, G and modified forms thereof; basic amino acid residues including K, R, H and modified forms thereof; and amide containing amino acid residues including N, Q and modified forms thereof
  MOD_RES 29: note = X is selected from basic amino acid residues including K, R, H and modified forms thereof; aromatic amino acid residues including F, Y, W and modified forms thereof; and hydrophobic amino acid residues including V, L, I, Nle and modified forms thereof
  MOD_RES 31: note = X is absent or is selected from any amino acid residue
  MOD_RES 32: note = X is absent or is selected from any amino acid residue
  MOD_RES 33: note = X is absent or is selected from any amino acid residue
  MOD_RES 34: note = X is absent or is selected from any amino acid residue
  MOD_RES 35: note = X is absent or is selected from any amino acid residue
XXXXCXXXXXXCXXDSDCXXXCXCXXXXXCXXXXX

SEQ ID NO: 172 - protein, 26 aa, synthetic construct
  MOD_RES 7: note = X is selected from L, V, T, I and modified forms of any of the foregoing amino acids
  MOD_RES 9: note = X is selected from R, W, V, T, S, Q, N, M, Nle, L, K, I, F, E, D, A and modified forms of any of the foregoing amino acids
  MOD_RES 10: note = X is selected from R, Y, V, T, Q, M, Nle, L, K, I, H, F, E, A and modified forms of any of the foregoing amino acids
  MOD_RES 11: note = X is selected from D, T, N, H and modified forms of any of the foregoing amino acids
  MOD_RES 12: note = X is selected from S, T, A and modified forms of any of the foregoing amino acids
  MOD_RES 13: note = X is selected from D, E and modified forms of any of the foregoing amino acids
  MOD_RES 15: note = X is selected from P, Y, M, Nle, L, I, F and modified forms of any of the foregoing amino acids
  MOD_RES 19: note = X is selected from I, V, K and modified forms of any of the foregoing amino acids
  MOD_RES 22: note = X is selected from A, V, T, S, R, P, K and modified forms of any of the foregoing amino acids
  MOD_RES 24: note = X is selected from R, K, H, G and modified forms of any of the foregoing amino acids
CPRIGRXCXXXXXCXGACXCRXTXFC

SEQ ID NO: 173 - protein, 35 aa, synthetic construct
  MOD_RES 1: note = X is absent or is selected from any amino acid residue
  MOD_RES 2: note = X is absent or is selected from any amino acid residue
  MOD_RES 3: note = X is absent or is selected from any amino acid residue
  MOD_RES 4: note = X is absent or is selected from any amino acid residue
  MOD_RES 11: note = X is selected from L, V, T, I and modified forms of any of the foregoing amino acids
  MOD_RES 13: note = X is selected from R, W, V, T, S, Q, N, M, Nle, L, K, I, F, E, D, A and modified forms of any of the foregoing amino acids
  MOD_RES 14: note = X is selected from R, Y, V, T, Q, M, Nle, L, K, I, H, F, E, A and modified forms of any of the foregoing amino acids
  MOD_RES 15: note = X is selected from D, T, N, H and modified forms of any of the foregoing amino acids
  MOD_RES 16: note = X is selected from S, T, A and modified forms of any of the foregoing amino acids
  MOD_RES 17: note = X is selected from D, E and modified forms of any of the foregoing amino acids
  MOD_RES 19: note = X is selected from P, Y, M, Nle, L, I, F and modified forms of any of the foregoing amino acids
  MOD_RES 23: note = X is selected from I, V, K and modified forms of any of the foregoing amino acids
  MOD_RES 26: note = X is selected from A, V, T, S, R, P, K and modified forms of any of the foregoing amino acids
  MOD_RES 28: note = X is selected from R, K, H, G and modified forms of any of the foregoing amino acids
  MOD_RES 31: note = X is absent or is selected from any amino acid residue
  MOD_RES 32: note = X is absent or is selected from any amino acid residue
  MOD_RES 33: note = X is absent or is selected from any amino acid residue
  MOD_RES 34: note = X is absent or is selected from any amino acid residue
  MOD_RES 35: note = X is absent or is selected from any amino acid residue
XXXXCPRIGRXCXXXXXCXGACXCRXTXFCXXXXX

SEQ ID NO: 174 - protein, 28 aa, synthetic construct
  MOD_RES 1: note = X is absent or is selected from at least one of a proteinaceous moiety consisting of from about 1 to about 50 amino acid residues (and all integer residues in between), and a protecting moiety
  MOD_RES 8: note = X is selected from L, V, T, I and modified forms of any of the foregoing amino acids
  MOD_RES 10: note = X is selected from R, W, V, T, S, Q, N, M, Nle, L, K, I, F, E, D, A and modified forms of any of the foregoing amino acids
  MOD_RES 11: note = X is selected from R, Y, V, T, Q, M, Nle, L, K, I, H, F, E, A and modified forms of any of the foregoing amino acids
  MOD_RES 12: note = X is selected from D, T, N, H and modified forms of any of the foregoing amino acids
  MOD_RES 13: note = X is selected from S, T, A and modified forms of any of the foregoing amino acids
  MOD_RES 14: note = X is selected from D, E and modified forms of any of the foregoing amino acids
  MOD_RES 16: note = X is selected from P, Y, M, Nle, L, I, F and modified forms of any of the foregoing amino acids
  MOD_RES 20: note = X is selected from I, V, K and modified forms of any of the foregoing amino acids
  MOD_RES 23: note = X is selected from A, V, T, S, R, P, K and modified forms of any of the foregoing amino acids
  MOD_RES 25: note = X is selected from R, K, H, G and modified forms of any of the foregoing amino acids
  MOD_RES 28: note = X is absent or is selected from at least one of a proteinaceous moiety consisting of from about 1 to about 50 amino acid residues (and all integer residues in between), and a protecting moiety
XCPRIGRXCXXXXXCXGACXCRXTXFCX

SEQ ID NO: 175 - protein, 11 aa, synthetic construct
  MOD_RES 2: note = X is 1 to 10 amino acid residues, wherein each amino acid residue is selected from any amino acid residue
  MOD_RES 4: note = X is 1 to 10 amino acid residues, wherein each amino acid residue is selected from any amino acid residue
  MOD_RES 6: note = X is 1 to 10 amino acid residues, wherein each amino acid residue is selected from any amino acid residue
  MOD_RES 8: note = X is 1 to 10 amino acid residues, wherein each amino acid residue is selected from any amino acid residue
  MOD_RES 10: note = X is 1 to 10 amino acid residues, wherein each amino acid residue is selected from any amino acid residue
CXCXCXCXCXC

SEQ ID NO: 176 - protein, 12 aa, synthetic construct
  MOD_RES 2: note = X is 1 to 10 amino acid residues, wherein each amino acid residue is selected from any amino acid residue
  MOD_RES 4: note = X is 1 to 10 amino acid residues, wherein each amino acid residue is selected from any amino acid residue
  MOD_RES 6: note = X is 1 to 10 amino acid residues, wherein each amino acid residue is selected from any amino acid residue
  MOD_RES 8: note = X is 1 to 10 amino acid residues, wherein each amino acid residue is selected from any amino acid residue
  MOD_RES 10: note = X is 1 to 10 amino acid residues, wherein each amino acid residue is selected from any amino acid residue
  MOD_RES 12: note = X is 1 to 10 amino acid residues, wherein each amino acid residue is selected from any amino acid residue
  SITE 1: note = N-terminus is amide bonded to the C-terminus of the C-terminal amino acid residue
  SITE 12: note = C-terminus is amide bonded to the N-terminus of the N-terminal cysteine
CXCXCXCXCXCX

(19) United States
(12) Patent Application Publication
(10) Pub. No.: US 2024/0344241 A1
(43) Pub. Date: Oct. 17, 2024

(54) PROTEINACEOUS MOLECULES AND USES THEREFOR

(71) Applicants: THE UNIVERSITY OF QUEENSLAND, Queensland (AU); The University of Tokyo, Tokyo (JP)

(72) Inventors: David James CRAIK, Queensland (AU); Simon John DE VEER, Queensland (AU); Yen-Hua HUANG, Queensland (AU); Joakim Erik SWEDBERG, Queensland (AU); Hiroaki SUGA, Tokyo (JP); Wenyu LIU, Tokyo (JP); Toby PASSIOURA, Tokyo (JP)

(21) Appl. No.: 18/701,094

(22) PCT Filed: Oct. 14, 2022
(86) PCT No.: PCT/AU2022/051238

§ 371 Date: Apr. 12, 2024

(30) Foreign Application Priority Data
Oct. 14, 2021 (AU) 2021903307

(51) Int. Cl. C40B 30/04; C12N 15/10; C12Q 1/02; C12R 1/19; G01N 33/68

(52) U.S. Cl. CPC C40B 30/04; C12N 15/1062; C12Q 1/02; G01N 33/6845; C12R 2001/19; G01N 2500/04

(57) ABSTRACT

Disclosed are proteinaceous coagulation factor XIIa (FXIIa) inhibitors and their use for treating or inhibiting the development of a condition in which inhibiting FXIIa stimulates or effects treatment or inhibition of the development of the condition. Suitable conditions include thromboembolism-associated conditions such as acute coronary syndrome, stroke, deep vein thrombosis and pulmonary embolism, a thrombosis, a thrombosis-associated hematologic disorder such as sickle cell disease or thrombophilia, and an inflammatory condition or a condition related to the kallikrein-kinin system such as hereditary angioedema, multiple sclerosis, rheumatoid arthritis or lupus. The proteinaceous FXIIa inhibitors are also useful for treating or inhibiting thrombus and/or embolus formation. In vitro methods for identifying a disulfide rich peptide which binds to a target substance are also disclosed.

This application claims priority to Australian Provisional Patent Application No. 2021903307 entitled “Proteinaceous molecules and uses therefor” filed 14 Oct. 2021, the contents of which are incorporated herein by reference in their entirety.

### FIELD OF THE INVENTION

This invention relates generally to proteinaceous coagulation factor XIIa (FXIIa) inhibitors and their use for treating or inhibiting the development of a condition in which inhibiting FXIIa stimulates or effects treatment or inhibition of the development of the condition. Suitable conditions include thromboembolism-associated conditions such as acute coronary syndrome, stroke, deep vein thrombosis and pulmonary embolism, a thrombosis, a thrombosis-associated hematologic disorder such as sickle cell disease or thrombophilia, and an inflammatory condition or a condition related to the kallikrein-kinin system such as hereditary angioedema, multiple sclerosis, rheumatoid arthritis or lupus. The proteinaceous FXIIa inhibitors are also useful for treating or inhibiting thrombus and/or embolus formation.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Cyclotides are plant-derived head-to-tail cyclic peptides, which comprise a cystine knot motif wherein a ring formed by two of the disulfide bonds and the intervening sections of the peptide backbone is pierced by the third disulfide bond. Peptides comprising a cystine knot motif typically have high levels of chemical, thermal and proteolytic stability, which may be advantageous for therapeutic use. Indeed, some cyclotides have been shown to be orally bioactive and/or able to penetrate cells. Cyclotides have also been shown to exert potent biological effects, which makes them appealing scaffolds for therapeutic development. However, difficulties in development of cyclotide-based therapeutics have been encountered partly due to the complex nature of the cyclotide scaffold, which may hinder facile production and screening of engineered variants. As such, utilization of this scaffold has been limited.

Ischemic complications, such as myocardial infarction and stroke, are a major cause of death and disability. Typically, these ischemic events are caused by the rupture of an unstable atherosclerotic plaque, leading to exposure of thrombogenic material and the acute formation of vessel occluding thrombi. If circulation is not restored promptly, oxygen and nutrient deprivation, as well as the build-up of metabolic waste products will quickly lead to muscle damage and tissue death. While treatment such as percutaneous coronary intervention (PCI) is available and often successful in restoring blood flow, the risk of recurrent cardiovascular events remains high even under optimal medication. Furthermore, paradoxically early restoration of blood flow causes a localized overshooting inflammatory response, thereby resulting in substantial cardiac tissue damage, described with the term ischemia/reperfusion injury. Strategies to reduce the risk of recurrent events consist among other medications of single or combined anti-platelet and anti-coagulant therapies. However, current therapies carry a substantial risk of major bleeding. Accordingly, new therapies with an improved safety profile are needed.

Coagulation factor XIIa (FXIIa) is a serine protease that initiates the intrinsic pathway of the coagulation system via coagulation factor XI (FXI) activation and also plays a role in the kallikrein-kinin system through prekallikrein activation. FXIIa has recently been identified as a promising target for the development of therapies for conditions associated with thrombus and/or embolus formation and inflammatory conditions. FXIIa is a particularly attractive target for therapeutic development, as FXIIa deficiency is not associated with a bleeding disorder, which suggests that targeting FXIIa could lead to the development of therapeutics with an improved safety profile that affect thrombosis without influencing hemostasis. FXIIa is also a target for development of therapeutics which treat inflammatory conditions, such as hereditary angioedema.

Accordingly, there is an unmet medical need for improved therapeutic agents that can be used for treating conditions that require inhibition of thrombus and/or embolus formation, and inflammatory conditions associated with FXIIa activity.

### SUMMARY OF THE INVENTION

The present invention is predicated in part on the design and discovery of proteinaceous molecules derived from the cyclotide, Momordica cochinchinensis trypsin inhibitor-II (MCoTI-II) that inhibit FXIIa activity. Notably, these proteinaceous molecules have high affinity for FXIIa and/or are selective for FXIIa over one or more other serine proteases, such as trypsin. Accordingly, the inventors have conceived that the proteinaceous molecules may be useful for treating or inhibiting the development of a condition associated with FXIIa activity, including thromboembolism-associated conditions such as acute coronary syndrome, stroke, deep vein thrombosis and pulmonary embolism, a thrombosis, a thrombosis-associated hematologic disorder such as sickle cell disease or thrombophilia, or an inflammatory condition or a condition related to the kallikrein-kinin system such as hereditary angioedema, multiple sclerosis, rheumatoid arthritis or lupus, as well as for treating or inhibiting thrombus and/or embolus formation.

Accordingly, in one aspect, there is provided a proteinaceous molecule comprising an amino acid sequence represented by Formula I:

CX₁X₂X₃X₄X₅X₆CX₇X₈DSDCPGACICX₉X₁₀X₁₁X₁₂X₁₃C (I)

wherein:
X₁ is selected from P and modified forms thereof; C and modified forms thereof; and F and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; hydrophobic amino acid residues including V, L, I, Ne and modified forms thereof; and acidic amino acid residues including D, E, hGlu and modified forms thereof;
X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; basic amino acid residues including K, R, H and modified forms thereof; and amide containing amino acid residues including N, Q and modified forms thereof;
X₆ is selected from any amino acid residue;
X₇ is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof; and hydrophobic amino acid residues including V, L, I, Ne and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof;
wherein the proteinaceous molecule is other than a proteinaceous molecule comprising or consisting of the amino acid sequence of any one of SEQ ID NOs: 1 to 7:

| [SEQ ID NO: 1] | |
|---|---|
| | CPKILKKCRRDSDCPGACICRGNGYC; |
| [SEQ ID NO: 2] | |
| | CPKILQRCRRDSDCPGACICRGNGYC; |
| [SEQ ID NO: 3] | |
| | CPRILKKCRRDSDCPGACICRGNGYC; |
| [SEQ ID NO: 4] | |
| | CPKILQRCRRDSDCPGACICLGNGYC; |
| [SEQ ID NO: 5] | |
| | CPKILKKCRHDSDCPGACICRGNGYC; |
| [SEQ ID NO: 6] | |
| | CFRILKKCRRDSDCPGACICRGNGYC; |
| | or |
| [SEQ ID NO: 7] | |
| | CFRIWKKCRRDSDCPGACICRGNGYC. |

In some embodiments, X₇ is selected from basic amino acid residues including K, R, H and modified forms thereof; amide containing amino acid residues including N, Q and modified forms thereof; and small amino acid residues including S, T, A, G and modified forms thereof.

In another aspect, there is provided a proteinaceous molecule comprising an amino acid sequence represented by Formula I:

CX₁X₂X₃X₄X₅X₆CX₇X₈DSDCPGACICX₉X₁₀X₁₁X₁₂X₁₃C (I)

wherein:
X₁ is selected from P and modified forms thereof; C and modified forms thereof; and F and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; hydrophobic amino acid residues including V, L, I, Ne and modified forms thereof; and acidic amino acid residues including D, E, hGlu and modified forms thereof;
X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; basic amino acid residues including K, R, H and modified forms thereof; and amide containing amino acid residues including N, Q and modified forms thereof;
X₆ is selected from any amino acid residue;
X₇ is selected from basic amino acid residues including K, R, H and modified forms thereof; amide containing amino acid residues including N, Q and modified forms thereof; and small amino acid residues including S, T, A, G and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Nle and modified forms thereof; wherein the proteinaceous molecule is other than a proteinaceous molecule comprising or consisting of the amino acid sequence of any one of SEQ ID NOs: 1 to 7:

| [SEQ ID NO: 1] | |
|---|---|
| | CPKILKKCRRDSDCPGACICRGNGYC; |
| [SEQ ID NO: 2] | |
| | CPKILQRCRRDSDCPGACICRGNGYC; |
| [SEQ ID NO: 3] | |
| | CPRILKKCRRDSDCPGACICRGNGYC; |
| [SEQ ID NO: 4] | |
| | CPKILQRCRRDSDCPGACICLGNGYC; |
| [SEQ ID NO: 5] | |
| | CPKILKKCRHDSDCPGACICRGNGYC; |
| [SEQ ID NO: 6] | |
| | CFRILKKCRRDSDCPGACICRGNGYC; |
| | or |
| [SEQ ID NO: 7] | |
| | CFRIWKKCRRDSDCPGACICRGNGYC. |

In some embodiments, X₁ is P or C; X₂ is R, G or K, especially R; X₃ is I, L, V, F, G, Nle, 4-F-Phe or 4-Me-Phe; X₄ is G, L, E, Y, V, W or Nle; X₅ is selected from aromatic amino acid residues including F, Y, W and modified forms thereof, hydrophobic amino acid residues including V, L, I, Nle and modified forms thereof, and basic amino acid residues including K, R, H and modified forms thereof, especially wherein X₅ is R, K, V, W or L; X₆ is selected from small amino acid residues including S, T, A, G and modified forms thereof, aromatic amino acid residues including F, Y, W and modified forms thereof, hydrophobic amino acid residues including V, L, I, Nle and modified forms thereof, and basic amino acid residues including K, R, H and modified forms thereof, especially wherein X₆ is K, L, Y, W, R, A or V; X₇ is K or R; X₈ is K or R; X₉ is R, I, A, Y or V; X₁₀ is G, A, R, P or F; X₁₁ is N, T, R, G or K; X₁₂ is G, R, T or K; and/or X₁₃ is Y, F, L, W or H.

In particular embodiments, X₁ is P; X₂ is R; X₃ is I, F, L, V or 4-F-Phe; X₄ is L, E, Nle, V, W or G; X₅ is K, R, V or W; X₆ is K, L, Y, W, R or A; X₇ is R or K; X₈ is R or K; X₉ is R, I, A or Y; X₁₀ is G, A, R or P; X₁₁ is N, T, R or G; X₁₂ is R, T, G or K; and X₁₃ is Y, F, L or W.

In further embodiments, X₁ is P; X₂ is R; X₃ is I, F, or 4-F-Phe; X₄ is L, E, Nle, V, W or G; X₅ is K or R; X₆ is K, L or A; X₇ is R or K; X₈ is R or K; X₉ is R; X₁₀ is G or A; X₁₁ is N or T; X₁₂ is R or G; and X₁₃ is Y or F.

In some embodiments, the proteinaceous molecule comprises, consists or consists essentially of an amino acid sequence represented by any one of SEQ ID NOs: 8-36:

[SEQ ID NO: 8]

DGGICPRIGRLCRRDSDCPGACICRATRFCGSGY;

[SEQ ID NO: 9]

GGICPRIGRLCRRDSDCPGACICRATRFCGSGYD;

[SEQ ID NO: 10]

GGICPRIGRLCRRDSDCPGACICRATRFCGSGSD;

[SEQ ID NO: 11]

DGGICPRILVYCRRDSDCPGACICIRRTYCGSGS;

[SEQ ID NO: 12]

GGICPRILVYCRRDSDCPGACICIRRTYCGSGSD;

[SEQ ID NO: 13]

DGGRCPRLLRWCRRDSDCPGACICARGGLCGSGS;

[SEQ ID NO: 14]

GGRCPRLLRWCRRDSDCPGACICARGGLCGSGSD;

[SEQ ID NO: 15]

DGGVCPRVGWRCRRDSDCPGACICYPTKWCGSGS;

[SEQ ID NO: 16]

GGVCPRVGWRCRRDSDCPGACICYPTKWCGSGSD;

[SEQ ID NO: 17]

DGGRCCGGYLVCRRDSDCPGACICVFKKHCGSGS;

[SEQ ID NO: 18]

GGRCCGGYLVCRRDSDCPGACICVFKKHCGSGSD;

[SEQ ID NO: 19]

DGGICPRIGRLCRRDSDCPGACICRGNGYCGSGS;

[SEQ ID NO: 20]

GGICPRIGRLCRRDSDCPGACICRGNGYCGSGSD;

[SEQ ID NO: 21]

DGGVCPKILKKCRRDSDCPGACICRATRFCGSGS;

[SEQ ID NO: 22]

GGVCPKILKKCRRDSDCPGACICRATRFCGSGSD;

[SEQ ID NO: 23]

RICPRIGRLCRRDSDCPGACICRATRFCG;

[SEQ ID NO: 24]

GGICPRIGRLCKRDSDCPGACICRATRFCGSGSD;

[SEQ ID NO: 25]

GGICPRIGRLCRKDSDCPGACICRATRFCGSGSD;

[SEQ ID NO: 26]

GGICPRIGRLCRRDSDCPGACICRATRFCGSGKD;

[SEQ ID NO: 27]

GGICPRFGRLCRRDSDCPGACICRATRFCGSGSD;

[SEQ ID NO: 28]

GGRCPRIGRLCRRDSDCPGACICRATRFCGSGSD;

[SEQ ID NO: 29]

RVCPR[4-F-Phe]EKKCRRDSDCPGACICRGNGYCG;

[SEQ ID NO: 30]

RVCPR[4-F-Phe]VKKCRRDSDCPGACICRGNGYCG;

[SEQ ID NO: 31]

RVCPR[4-F-Phe]WKKCRRDSDCPGACICRGNGYCG;

[SEQ ID NO: 32]

RVCPR[4-F-Phe]ERKCRRDSDCPGACICRGNGYCG;

[SEQ ID NO: 33]

RVCPR[4-F-Phe]VRKCRRDSDCPGACICRGNGYCG;

[SEQ ID NO: 34]

RVCPR[4-F-Phe]WRKCRRDSDCPGACICRGNGYCG;

[SEQ ID NO: 35]

RVCPR[4-F-Phe][Nle]KACRRDSDCPGACICRGNGYCG;
or

[SEQ ID NO: 36]

GGVCPR[4-F-Phe]EKKCRRDSDCPGACICRGNGYCGSGSD.

In particular embodiments, the proteinaceous molecule comprises, consists or consists essentially of an amino acid sequence represented by SEQ ID NO: 8 or 19.

In another aspect, there is provided a proteinaceous molecule comprising, consisting or consisting essentially of an amino acid sequence represented by Formula VII:

CPRIGRX₆CX₇X₈X₂₇X₂₈X₂₉CX₂₃GACX₂₆CRX₁₀TX₁₂FC (VII)

wherein:
X₆ is selected from L, V, T, I and modified forms of any of the foregoing amino acids;
X₇ is selected from R, W, V, T, S, Q, N, M, Nle, L, K, I, F, E, D, A and modified forms of any of the foregoing amino acids;
X₈ is selected from R, Y, V, T, Q, M, Nle, L, K, I, H, F, E, A and modified forms of any of the foregoing amino acids;
X₂₇ is selected from D, T, N, H and modified forms of any of the foregoing amino acids;
X₂₈ is selected from S, T, A and modified forms of any of the foregoing amino acids;
X₂₉ is selected from D, E and modified forms of any of the foregoing amino acids;
X₂₃ is selected from P, Y, M, Nle, L, I, F and modified forms of any of the foregoing amino acids;
X₂₆ is selected from I, V, K and modified forms of any of the foregoing amino acids;
X₁₀ is selected from A, V, T, S, R, P, K and modified forms of any of the foregoing amino acids; and
X₁₂ is selected from R, K, H, G and modified forms of any of the foregoing amino acids.

While both cyclic and acyclic molecules are contemplated, in particular embodiments the proteinaceous molecule is a cyclic molecule, especially wherein the proteinaceous molecule is cyclized through N-to-C cyclization.

In some embodiments, the six cysteine residues in the proteinaceous molecule are bonded in pairs to form three disulfide bonds. In particular embodiments, the disulfide bonds are formed between the side chains of Cys 1 and Cys 18, Cys 8 and Cys 20, and Cys 14 and Cys 26 (numbered in accordance with Formula I) (i.e. Cys I and Cys IV, Cys II and Cys V, and Cys III and Cys VI).

In another aspect, there is provided a composition comprising, consisting or consisting essentially of a proteinaceous molecule of the invention and a pharmaceutically acceptable carrier or diluent.

Further provided herein, in another aspect, is a method of treating or inhibiting the development of a condition in which inhibiting FXIIa activity is associated with effective treatment or inhibition, comprising administering the proteinaceous molecule of the invention.

In particular embodiments, the condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, sickle cell disease, thrombophilia, and thrombosis resulting from a medical implant, device or extracorporeal circulation procedure in which blood is exposed to an artificial surface that promotes thrombosis.

In some embodiments, the condition is an inflammatory condition, such as hereditary angioedema, anaphylaxis, rheumatoid arthritis, pancreatitis, sepsis, multiple sclerosis or lupus.

In another aspect, there is provided a method of inhibiting an activity of FXIIa, comprising contacting FXIIa with a proteinaceous molecule of the invention.

In a further aspect, there is provided a method of treating or inhibiting the development of thrombosis in a subject, comprising administering a proteinaceous molecule of the invention to the subject.

Also provided is a method of inhibiting coagulation in a subject, comprising administering a proteinaceous molecule of the invention to the subject.

In another aspect, there is provided a method for inhibiting thrombus or embolus formation in a subject, comprising administering the proteinaceous molecule of the invention to the subject to thereby inhibit thrombus or embolus formation in the subject.

Further provided is a method for treating or inhibiting the development of a thromboembolism-associated condition in a subject, comprising administering the proteinaceous molecule of the invention to the subject.

Suitable thromboembolism-associated conditions include an arterial cardiovascular thromboembolic disorder, a venous cardiovascular or cerebrovascular thromboembolic disorder and a thromboembolic disorder in a chamber of the heart or in the peripheral circulation. In some embodiments, the thromboembolism-associated condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from a medical implant, device or extracorporeal circulation (extracorporeal membrane oxygenation (ECMO), cardiopulmonary bypass) procedure in which blood is exposed to an artificial surface that promotes thrombosis. The medical implant or device may, in some embodiments, be selected from a prosthetic valve, artificial valve, indwelling catheter, stent, blood oxygenator, shunt, vascular access port, ventricular assist device and artificial heart or heart chamber, and vessel graft. Suitable procedures include, for example, a cardiopulmonary bypass, percutaneous coronary intervention and hemodialysis.

In particular embodiments, the thromboembolism-associated condition is selected from acute coronary syndrome, stroke, deep vein thrombosis and pulmonary embolism.

In still another aspect, there is provided a method for treating or inhibiting the development of a thrombosis-associated hematologic disorder in a subject, comprising administering the proteinaceous molecule of the invention to the subject.

In some embodiments, the hematologic disorder is sickle cell disease or thrombophilia.

In another aspect, there is provided an in vitro method for identifying a disulfide rich peptide which binds to a target substance comprising:
a) preparing an mRNA library based on a disulfide rich peptide scaffold;
b) ligating mRNA in the library to puromycin to form mRNA-puromycin conjugates;
c) translating the mRNA-puromycin conjugates using a prokaryotic translation system to produce mRNA-puromycin-peptide conjugates;
d) reverse transcribing the conjugates to form mRNA:cDNA-puromycin-peptide conjugates;
e) performing affinity selection against the target substance to select for mRNA:cDNA-puromycin-peptide conjugates that bind to the target substance;
f) performing nucleic acid amplification on the cDNA of the selected mRNA:cDNA-puromycin-peptide conjugates to generate an enriched cDNA library; and
g) sequencing the enriched cDNA library to identify a disulfide rich peptide which binds to the target substance.

In some embodiments, the disulfide rich peptide contains at least six cysteine residues. In such embodiments, the disulfide rich peptide contains at least three disulfide bonds. In particular embodiments, the disulfide rich peptide contains a cystine knot motif.

In some embodiments, the disulfide rich peptide has at least about 2-fold greater binding affinity for the target substance than the disulfide rich peptide scaffold. In some embodiments, the disulfide rich peptide has at least about 2-fold greater selectivity for the target substance than the disulfide rich peptide scaffold.

In particular embodiments, the prokaryote is Escherichia coli.

In some embodiments, the prokaryotic translation system does not comprise release factor 1 (RF1).

In some embodiments, the prokaryotic translation system comprises tRNAs, initiation factors, elongation factors, release factors, T7 RNA polymerase, nucleoside triphosphates, aminoacyl-tRNA synthetases (ARS), ribosomes and the 20 natural amino acids. In particular embodiments, the tRNAs, initiation factors, elongation factors and/or release factors are from E. coli.

In specific embodiments, the prokaryotic translation system comprises E. coli ribosome, initiation factor 1 (IF1), initiation factor 2 (IF2), initiation factor 3 (IF3), elongation factor G (EF-G), elongation factor thermo unstable (EF-Tu), elongation factor thermo stable (EF-Ts), release factor 2 (RF2), release factor 3 (RF3), ribosome release factor (RRF), alanyl-tRNA synthetase (AlaRS), arginyl-tRNA synthetase (ArgRS), asparaginyl-tRNA synthetase (AsnRS), aspartyl-tRNA synthetase (AspRS), cysteinyl-tRNA synthetase (CysRS), glutamyl-tRNA synthetase (GluRS), glutaminyl-tRNA synthetase (GlnRS), glycyl-tRNA synthetase (GlyRS), histidyl-tRNA synthetase (HisRS), isoleucyl-tRNA synthetase (IleRS), leucyl-tRNA synthetase (LeuRS), lysyl-tRNA synthetase (LysRS), methionyl-tRNA synthetase (MetRS), phenylalanyl-tRNA synthetase (PheRS), prolyl-tRNA synthetase (ProRS), seryl-tRNA synthetase (SerRS), threonyl-tRNA synthetase (ThrRS), tryptophanyl-tRNA synthetase (TrpRS), tyrosyl-tRNA synthetase (TyrRS), valyl-tRNA synthetase (VaIRS), methionyl-tRNA formyltransferase (MTF), T7 RNA polymerase, E. coli total tRNA, adenosine triphosphate (ATP), guanosine triphosphate (GTP), cytidine triphosphate (CTP) and uridine triphosphate (UTP) and the 20 natural amino acids. In some embodiments, the translation system further comprises inorganic pyrophosphatase, nucleoside diphosphate kinase, creatine phosphate, 10-formyl-5,6,7,8-tetrahydrofolic acid, spermidine, dithiothreitol (DTT), potassium acetate, magnesium acetate, HEPES-KOH buffer, myokinase and creatine kinase.

In some embodiments, prior to step g), an mRNA library is prepared based on the enriched cDNA library produced in step f), and steps b) to f) are repeated. In particular embodiments, this process is repeated a further two times for a total of four rounds of selection.

### BRIEF DESCRIPTION OF THE DRAWINGS

FIG. 1 is an image illustrating the structure of MCoTI-II and the strategy for mRNA display. FIG. 1A shows the structure of the prototypic trypsin inhibitor cyclotide MCoTI-II (PDB 4GUX) showing the head-to-tail cyclic backbone and knotted arrangement of three disulfide bonds deriving from six conserved Cys residues (labelled I-VI). Backbone regions between the Cys residues are referred to as loops. FIG. 1B is a schematic illustration of mRNA display strategy for the discovery of FXIIa inhibitors based on the MCoTI-II scaffold whereby a single Val residue in loop 6, and all of loops 1 and 5 are varied (Pu=puromycin). FIG. 1C displays the sequence of native MCoTI-II showing the disulfide connectivity (black lines) and head-to-tail cyclic backbone (thick grey line). Key contact residues P4-P1 and P1′-P4′ sites (Schechter-Berger nomenclature) are indicated above the sequence. The lower sequence shows the regions of sequence varied in the display library (indicated by X).

FIG. 2 is a schematic illustration of the mRNA display approach used. In brief, a DNA library assembled from synthetic oligonucleotides was transcribed into mRNA and ligated to puromycin at the 3′ end. In vitro translation of this library led to the formation of an acyclic MCoTI-II-based peptide library in which each peptide was covalently linked to its cognate mRNA through the puromycin moiety, which was then reverse transcribed to generate mRNA:cDNA-peptide conjugates. Affinity selection was conducted against biotinylated human β-FXIIa embedded on magnetic dynabeads and an enriched DNA library was recovered by PCR. The whole process was repeated until increased rates of target binding were observed. Deconvolution of the library was achieved through sequencing of the final (and intermediate) enriched cDNA libraries.

FIG. 3 is a sequence alignment of the sequences of the randomized region in the top 19 most abundant peptides recovered from affinity selection against FXIIa. The right column population (%) indicates the proportion of each sequence in the total recovered library. The sequence of MCoTI-II is shown above the selected peptides. The lower numbers indicate the position of the residues in the peptide.

FIG. 4 is the 1D ¹H-NMR spectra of chemically synthesized cyclic MCoTI-II and acyclic and cyclic MCoFx1-5.

FIG. 5 is a graph showing the α-proton secondary chemical shifts analysis of MCoTI-II, cMCoFx1 and loop-replacing variants cMCoTI-fxL1 and cMCoTI-fxL5. The dotted lines represent secondary chemical shift values of −0.1 and 0.1 ppm. Sequences of the four peptides are shown below the chart. The regions identical to cMCoFx1 are loop 1 in cMCoTI-fxL1 and loop 5 in cMCoTI-fxL5. Six cysteines are highlighted, indicating the arrangement of three disulfide bonds.

FIG. 6 is a graph showing the cytotoxicity of MCoTI-II and cyclic MCoFx1 against human umbilical vein endothelial cells (HUVECs).

FIG. 7 is a graph of the inhibitory activity of (a) cMCoFx1 and (b) cMCoTI-fxL1 in activated partial thromboplastin time (aPTT) assays that measure clotting via the intrinsic pathway. The concentration of inhibitor required to double the clotting time observed in control assays (44.3 s, grey dashed line, buffer replaces addition of inhibitor) is shown as EC_{2×}.

FIG. 8 is a graph of the inhibitory activity measurement of cMCoFx1 and cMCoTI-fxL1 at concentrations of 5 μM and 10 μM in prothrombin time (PT) assays, which measure clotting via the extrinsic pathway. The control bar indicates the clotting time where buffer replaces addition of inhibitors.

FIG. 9 is a graph of the (a) stability of MCoTI-II, aMCoFx1, cMCoFx1, and the loop-grafted variant cMCoTI-fxL1 in human serum. Control indicates a linear peptide with sequence of EAIYAAPFAKKK which was fully degraded within 1 h. Time courses represent the percentage of peptide remaining after incubation in 100% human serum at 37° C. for up to 24 h. Results are the mean±SEM from three replicates. The activity of human serum after incubation at 37° C. for up to 24 h is verified in (b). Human serum was incubated for 0, 4, or 24 h (indicated at the top of the graph), and the percentage of control peptide remaining was measured at 0, 1, or 2 h after peptide addition.

FIG. 10 is a series of bar graphs showing the inhibitory activity of MCoTI-II variants against FXIIa, trypsin, matriptase and kallikrein-related peptidase 4 (KLK4) in a competitive inhibition assay, wherein the residues in position P1′, P2′, P3′ and P4′ (indicated at the top of the figure) were substituted with the residues indicated on the x-axis (wherein Ne=norleucine). The concentration of inhibitor was fixed for each protease (FXIIa: 25 nM, trypsin: 10 nM, matriptase: 5 nM, KLK4: 1.25 nM).

FIG. 11 is a graph showing the inhibitory activity of MCoTI-II variants against FXIIa, trypsin, matriptase and KLK4, in a competitive inhibition assay, wherein the residues of MCoTI-II in position P1′, P2′ and P3′ were substituted with the residues indicated on the x-axis (wherein 4-Fl=4-fluoro-L-phenylalanine; 4-Me=4-methyl-L-phenylalanine; and hGlu=homoglutamic acid). The concentration of inhibitor was fixed for each protease (FXIIa: 25 nM, trypsin: 10 nM, matriptase: 5 nM, KLK4: 1.25 nM).

FIG. 12 is a graph showing the inhibitory activity of MCoTI-II variants, M, 1, 2, 3, 4, 5, 6 and 7, against FXIIa, trypsin, matriptase and KLK4, in a competitive inhibition assay. Peptide sequences are listed in Table 12. All peptides were tested at 25 nM.

FIG. 13 illustrates the activity of MCoTI-II variants, 1, 3, and 7 in comparison with the template MCoTI-II peptide, M (“temp”). FIG. 13A illustrates the residues in positions P1, P1′, P2′, P3′ and P4′ of the sequences; FIG. 13B provides the Ki values for the variants against FXIIa, trypsin, matriptase and KLK4 (where less than 50% inhibition was observed at 10 μM, “>10 μM” is listed); and FIG. 13C is a graph showing the inhibitory activity of MCoTI-II variant, 1, in an activated partial thromboplastin time (aPTT) assay that measures clotting via the intrinsic pathway, and a prothrombin time (PT) assay, which measures clotting via the extrinsic pathway. The control line (grey dashed line) indicates the clotting time where buffer replaces addition of inhibitors.

FIG. 14 illustrates the W-scores of the single-position mutants of MCoFx1 in the saturation mutagenesis study, where each residue of MCoFx1 was replaced with each of the 20 naturally occurring amino acid residues. FIG. 14A is a W-score map and FIG. 14B contains the corresponding W-score values.

FIG. 15 is a graph showing the activity of cyclic MCoFx7 (2.5, 5, 10 and 20 μM) in an activated clotting time assay using human whole blood. The dotted lines indicate the therapeutic range for patients on ECMO receiving the standard-of-care anticoagulant, heparin. Data points represent the mean±SEM (n=3).

FIG. 16 is a graph showing the anticoagulant activity (clotting time) of cyclic MCoFx7 (2.5, 5, 10 and 20 μM) using thromboelastometry (TEM) that was measured after activation of the intrinsic pathway (INTEM). Data points represent the mean±SEM (n=3).

FIG. 17 is a graph showing the activity of cyclic MCoFx7 compared to the standard of care, heparin, in an ex vivo extracorporeal membrane oxygenation (ECMO) model. Average blood flow, pump speed, heater temperature, pump pressure and delta oxygenator pressure (delta oxygenator P) are provided (FIG. 17A), together with the delta oxygenator P over time (FIG. 17B). Cyclic MCoFx7 maintained similar blood flow rate, pump speed and pump pressure to heparin, and had a stable delta oxygenator P.

FIG. 18 is a series of graphs showing the clotting time of blood samples containing cyclic MCoFx7 or heparin from the ex vivo extracorporeal membrane oxygenation (ECMO) model using an ACT assay (FIG. 18A), INTEM assay (INTEM-CT; FIG. 18B) and HEPTEM assay (HEPTEM-CT; FIG. 18C).

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same composition. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents.

Amino acid residues are referred to herein interchangeably using their full name or the one or three letter codes standard in the art. Abbreviations used for unnatural or modified amino acid residues or derivatives thereof are defined herein where appropriate.

Amino acid residues are defined herein on the basis of the side chain classification in some instances. Families of amino acid residues having similar side chains have been defined in the art, which can be generally sub-classified as follows:

TABLE 1
AMINO ACID SUB-CLASSIFICATION

| Sub-Classes | Amino Acids |
|---|---|
| Acidic | Aspartic acid and Glutamic acid |
| Basic | Noncyclic: Arginine and Lysine; Cyclic: Histidine |
| Charged | Aspartic acid, Glutamic acid, Arginine, Lysine and |
| | Histidine |
| Small | Glycine, Serine, Alanine, Threonine and Proline; |
| | especially Glycine, Serine, Alanine and Threonine |
| Polar/neutral | Asparagine, Histidine, Glutamine, Cysteine, |
| | Serine and Threonine |
| Polar/large | Asparagine and Glutamine |
| Hydrophobic | Tyrosine, Valine, Isoleucine, Leucine, Methionine, |
| | Phenylalanine and Tryptophan; especially Valine, |
| | Isoleucine and Leucine |
| Aromatic | Tryptophan, Tyrosine and Phenylalanine |
| Residues that | Glycine and Proline |
| influence chain | |
| orientation | |
| Amide | Asparagine and Glutamine |
| containing amino | |
| acid residues | |

As used herein, the term “and/or” refers to and encompasses any and all possible combinations of one or more of the associated listed items, as well as the lack of combinations when interpreted in the alternative (or).

The term “antagonist” and grammatical equivalents thereof as used herein refers to a molecule that partially or completely inhibits, by any mechanism, an effect of another molecule such as an enzyme, receptor or intracellular mediator. In the context of the present invention, the term “antagonist” refers to a molecule that is a direct antagonist that binds to or otherwise interacts with FXIIa, especially β-FXIIa, most especially human β-FXIIa. Antagonism of FXIIa may inhibit or reduce FXIIa activity and/or function, including any one or more of enzymatic activity (e.g. proteolytic activity), coagulation factor XI (FXI) activation, prekallikrein activation, plasminogen activation and a downstream activity thereof such as bradykinin release through the kallikrein-kinin system and thrombus formation through the coagulation system. By way of example, “antagonize” can refer to a decrease of about 10%, 20%, 30%, 40%, 50%, 60%, 70%, 80%, 90% or 100% in an activity, or function relative to the activity or function of FXIIa in the absence of the antagonist.

The term “anti-coagulant” refers to the effect of a moiety or agent, which reduces or inhibits coagulation of the blood. Anti-coagulant moieties and agents may have anti-platelet and/or anti-thrombotic activity.

The term “any amino acid residue” is used herein to refer to any of the 20 naturally occurring amino acid residues and modified versions thereof, including residues with modified side chains, N-methyl amino acids, α-methyl amino acids, residues with acetylated N-termini, beta amino acids, and the like.

The term “coagulation” or “blood clotting” as used herein refers to the process by which blood changes from a liquid to a gel. It potentially results in hemostasis, the cessation of blood loss from a damaged vessel, followed by repair.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified for the listed elements. Thus, the phrase “consisting essentially of” indicates that the listed elements are required or mandatory, but that other elements are optional and may or may not be present depending upon whether or not they affect the activity or action of the listed elements.

By “derivative” is meant a molecule, such as a polypeptide, that has been derived from the basic molecule by modification, for example by conjugation or complexing with other chemical moieties or by post-translational modification techniques as would be understood in the art. The term “derivative” also includes within its scope alterations that have been made to a parent molecule including additions or deletions that provide for functionally equivalent molecules.

As used herein, the term “dosage unit form” refers to physically discrete units suited as unitary dosages for the subject to be treated, each unit containing a predetermined quantity of active material calculated to produce the desired therapeutic effect in association with the required pharmaceutically acceptable vehicle.

By “effective amount”, in the context of treating or inhibiting the development of a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “embolus” (plural “emboli”), as used herein, refers to a gaseous, liquid or solid (e.g. particulate) matter that acts as a traveling “clot” and usually refers to any detached intravascular matter that is capable of occluding a vessel. The occlusion can occur at a site distant from the point of origin. The composition of an embolus includes, but is not limited to, bubbles or CO₂⁻; oil; fat; cholesterol; debris, such as vessel debris, e.g. calcifications, tissue, or tumor fragments; coagulated blood; an organism such as bacteria or a parasite, or other infective agent; or foreign material. The term “bubbles” includes an embolus formed of air or other gas, or in certain instances, a liquid that is not blood or coagulated blood. A bubble may be spherical or non-spherical in shape. The term “microembolus” is encompassed by the term “embolus” as used herein, and refers to an embolus of microscopic size and may be comprised of the same materials as an embolus as defined above. A common example of an embolus is a platelet aggregate dislodged from an atherosclerotic lesion. The dislodged platelet aggregate is transported by the bloodstream through the cerebrovasculature until it reaches a vessel too small for further propagation. The clot remains there, clogging the vessel and preventing blood flow from entering the distal vasculature. Emboli can originate from distant sources such as the heart, lungs, and peripheral circulation, which may eventually travel within the cerebral blood vessels, obstructing flow and causing stroke. Other sources of emboli include atrial fibrillation and valvular disease.

The terms “hematological disease” or “hematological disorders” are used interchangeably herein, and refer to disorders that primarily affect the cells of hematological origin, in common language denoted as cells of the blood.

As used herein, the phrase “inhibit the development of” refers to a prophylactic treatment which increases the resistance of a subject to developing the disease, disorder or condition or, in other words, decreases the likelihood that the subject will develop the disease, disorder or condition as well as a treatment after the disease, disorder or condition has begun in order to reduce or eliminate it altogether or prevent it from becoming worse. This phrase also includes within its scope preventing the disease, disorder or condition from occurring in a subject which may be predisposed to the disease, disorder or condition but has not yet been diagnosed as having it.

The term “inhibitor” as used herein refers to an agent that decreases or inhibits at least one function or biological activity of a target molecule. For example, an FXIIa inhibitor is an agent that inhibits at least one function or biological activity of FXIIa, such as any one or more of enzymatic activity (e.g. proteolytic activity), factor XI activation, prekallikrein activation, plasminogen activation and a downstream activity thereof, such as bradykinin release through the kallikrein-kinin system and thrombus formation through the coagulation system.

As used herein, the term “isolated” refers to material that is substantially or essentially free from components that normally accompany it in its native state. For example, an “isolated proteinaceous molecule” refers to in vitro isolation and/or purification of a proteinaceous molecule from its natural cellular environment and from association with other components of the cell. “Substantially free” means that a preparation of proteinaceous molecule is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, the preparation of proteinaceous molecule has less than about 30, 25, 20, 15, 10, 9, 8, 7, 6, 5, 4, 3, 2 or 1% (by dry weight), of molecules that are not the subject of this invention. When the proteinaceous molecule is recombinantly produced, it is also desirably substantially free of culture medium, i.e., culture medium represents less than about 20, 15, 10, 5, 4, 3, 2 or 1% of the volume of the preparation. The invention includes isolated or purified preparations of at least 0.01, 0.1, 1.0, and 10 milligrams in dry weight.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, fillers, detergents, coloring agents, wetting or emulsifying agents, pH buffering agents, preservatives and the like.

Similarly, a “pharmacologically acceptable” salt, ester, amide, prodrug or derivative of a compound as provided herein is a salt, ester, amide, prodrug or derivative that this not biologically or otherwise undesirable.

As used herein, the terms “polypeptide”, “proteinaceous molecule”, “peptide” and “protein” are used interchangeably to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations, attachment of lipid or protecting/stabilizing moieties and the like. Soluble forms of the subject proteinaceous molecules are particularly useful. Included within the definition are, for example, polypeptides containing one or more analogues of an amino acid including, for example, unnatural amino acids, polypeptides with substituted linkages and polypeptides with PEG groups and lipophilic moieties.

The terms “reduce”, “inhibit”, “decrease”, “prevent”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. When these terms are used to refer to the action of a molecule or agent, the first sample may be a sample in the presence of the molecule or agent and the second sample may be a comparative sample without the molecule or agent. In one embodiment, the reduction may be determined subjectively, for example when a patient refers to their subjective perception of disease symptoms, such as pain, shortness of breath, motor symptoms, etc. In another embodiment, the reduction may be determined objectively, for example when the size of a thrombus in a sample from a patient is smaller than in an earlier sample from the patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

As used herein, the terms “salts” and “prodrugs” include any pharmaceutically acceptable salt, ester, hydrate or any other compound which, upon administration to the recipient, is capable of providing (directly or indirectly) a proteinaceous molecule of the invention, or an active metabolite or residue thereof. The term “pharmaceutically acceptable salts” refers without limitation to derivatives of the disclosed compounds wherein the parent compound is modified by converting an existing acid or base moiety to its salt form (e.g. by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate and valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present invention include the conventional non-toxic salts of the parent compound formed, for example, from non-toxic inorganic or organic acids. The pharmaceutically acceptable salt can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in, for example, Remington (1985) Remington's Pharmaceutical Sciences, Mack Publishing Co., Easton, Pa., 17th edition; Stahl and Wermuth (2002) Pharmaceutical Salts: Properties, Selection, and Use, Wiley-VCH; and Berge et al. (1977) Journal of Pharmaceutical Science, 66: 1-19, each of which is incorporated herein by reference in its entirety.

The term “sequence identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity.

“Similarity” refers to the percentage number of amino acids that are identical or constitute conservative substitutions as defined in Tables 1 and 2 herein. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polypeptides include “reference sequence,” “comparison window”, “sequence identity,” “percentage of sequence identity” and “substantial identity”. A “reference sequence” is at least 20 but frequently 25 to 34 amino acid residues in length. As two amino acid sequences may each comprise (1) a sequence (i.e. only a portion of the complete proteinaceous molecule) that is similar between the two proteinaceous molecules, and (2) a sequence that is divergent between the two proteinaceous molecules, sequence comparisons between two (or more) proteinaceous molecules are typically performed by comparing sequences of the two proteinaceous molecules over a “comparison window” to identify and compare local regions of sequence similarity. A “comparison window” refers to a conceptual segment of at least 6 contiguous positions in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al. (1997) Nucl. Acids Res. 25: 3389. A detailed discussion of sequence analysis can be found in Unit 19.3 of Ausubel et al. (1994-1998) Current Protocols in Molecular Biology, John Wiley & Sons Inc, Chapter 15; Lambert et al. (2003) Current Genomics, 4:131-146; and Bawano et al. (2017) Bioinformatics, Volume 1: Data, Sequence Analysis and Evolution (Methods in Molecular Biology (1525)), Humana Press, pages 167-189.

The term “subject” as used herein refers to a vertebrate subject, particularly a mammalian or avian (bird) subject, for whom therapy or prophylaxis is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular embodiments, the subject is a primate, suitably a human. However, it will be understood that the aforementioned terms do not imply that symptoms are present.

The term “thrombosis” as used herein refers to the formation of a blood clot inside a blood vessel that obstructs the flow of blood through the circulatory system.

The term “thrombus” (plural “thrombi”) or “blood clot” as used herein refers to a solid or semi-solid mass formed from the constituents of blood within the vascular system that is the product of blood coagulation. There are two components to a thrombus, aggregated platelets that form a platelet plug, and a mesh of cross-linked fibrin protein.

The term “translation system” is used herein to refer to a composition comprising components which enable translation of an mRNA sequence. For example, a translation system may comprise tRNAs, initiation factors, elongation factors, release factors, RNA polymerase, nucleoside triphosphates, aminoacyl-tRNA synthetases (ARS), ribosomes and amino acids. A “prokaryotic translation system” refers to a composition comprising at least one prokaryotic component, such as prokaryotic tRNAs, ribosomes, initiation factors, elongation factors and/or release factors. In particular embodiments, the prokaryote is E. coli.

As used herein, the terms “treatment”, “treating”, and the like, refer to obtaining a desired pharmacologic and/or physiologic effect. The effect may be therapeutic in terms of a partial or complete cure for a disease, disorder or condition and/or adverse effect attributable to the disease, disorder or condition. These terms also cover any treatment of a condition or disease in a subject, particularly in a human, and include: (a) inhibiting the disease or condition, i.e. arresting its development; or (b) relieving the disease or condition, i.e. causing regression of the disease or condition.

Each embodiment described herein is to be applied mutatis mutandis to each and every embodiment unless specifically stated otherwise.

### 2. Abbreviations

The following abbreviations are used throughout the application:
Ac=Acetyl
PEG=Poly(ethylene glycol)
Nle=Norleucine
4-F-Phe=4-fluoro-L-phenylalanine
4-Me-Phe=4-methyl-L-phenylalanine
hGlu=Homoglutamic acid
equiv=Equivalents
mins=minutes
h or hr=hour

### 3. Proteinaceous Molecules

The present invention is based, in part on the finding that particular proteinaceous molecules derived from MCoTI-II inhibit FXIIa activity. Notably, these proteinaceous molecules have high potency and/or selectivity for FXIIa over one or more other serine proteases. Based on this finding, the inventors consider that the proteinaceous molecules may be useful for treating or inhibiting the development of a condition associated with FXIIa activity, including thromboembolism-associated conditions such as acute coronary syndrome, stroke, deep vein thrombosis and pulmonary embolism, a thrombosis, a thrombosis-associated hematologic disorder, such as sickle cell disease or thrombophilia, or an inflammatory condition or a condition related to the kallikrein-kinin system, such as hereditary angioedema, multiple sclerosis, rheumatoid arthritis or lupus, as well as for treating or inhibiting thrombus and/or embolus formation.

Accordingly, in one aspect, there is provided a proteinaceous molecule comprising an amino acid sequence represented by Formula I:

CX₁X₂X₃X₄X₅X₆CX₇X₈DSDCPGACICX₉X₁₀X₁₁X₁₂X₁₃C (I)

wherein:
X₁ is selected from P and modified forms thereof; C and modified forms thereof; and F and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; hydrophobic amino acid residues including V, L, I, Ne and modified forms thereof; and acidic amino acid residues including D, E, hGlu and modified forms thereof;
X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; basic amino acid residues including K, R, H and modified forms thereof; and amide containing amino acid residues including N, Q and modified forms thereof;
X₆ is selected from any amino acid residue;
X₇ is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof (e.g. E); and hydrophobic amino acid residues including V, L, I, Ne and modified forms thereof (e.g. V);
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof.

In particular embodiments:
X₁ is selected from P and modified forms thereof; C and modified forms thereof; and F and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; hydrophobic amino acid residues including V, L, I, Ne and modified forms thereof; and acidic amino acid residues including D, E, hGlu and modified forms thereof;
X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; basic amino acid residues including K, R, H and modified forms thereof; and amide containing amino acid residues including N, Q and modified forms thereof;
X₆ is selected from any amino acid residue;
X₇ is selected from basic amino acid residues including K, R, H and modified forms thereof; amide containing amino acid residues including N, Q and modified forms thereof; and small amino acid residues including S, T, A, G and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof.

In some embodiments, the proteinaceous molecule is other than a proteinaceous molecule comprising or consisting of the amino acid sequence of any one of SEQ ID NOs: 1 to 7:

| [SEQ ID NO: 1] | |
|---|---|
| | CPKILKKCRRDSDCPGACICRGNGYC; |
| [SEQ ID NO: 2] | |
| | CPKILQRCRRDSDCPGACICRGNGYC; |
| [SEQ ID NO: 3] | |
| | CPRILKKCRRDSDCPGACICRGNGYC; |
| [SEQ ID NO: 4] | |
| | CPKILQRCRRDSDCPGACICLGNGYC; |
| [SEQ ID NO: 5] | |
| | CPKILKKCRHDSDCPGACICRGNGYC; |
| [SEQ ID NO: 6] | |
| | CFRILKKCRRDSDCPGACICRGNGYC; |
| | or |
| [SEQ ID NO: 7] | |
| | CFRIWKKCRRDSDCPGACICRGNGYC. |

In some embodiments, when X₁ is P, X₂ is K, X₃ is I, X₄ is L, X₅ is K and X₆ is K; X₉ is other than R, X₁₀ is other than G, X₁₁ is other than N, X₁₂ is other than G and/or X₁₃ is other than Y. In alternative embodiments, the proteinaceous molecule does not comprise an amino acid sequence of any one of SEQ ID NOs: 37 to 42:

| [SEQ ID NO: 37] | |
|---|---|
| | PKILKK; |
| [SEQ ID NO: 38] | |
| | PKILQR; |
| [SEQ ID NO: 39] | |
| | PRILKK; |
| [SEQ ID NO: 40] | |
| | PRILKQ; |
| [SEQ ID NO: 41] | |
| | FRILKK; |
| | or |
| [SEQ ID NO: 42] | |
| | FRIWKK. |

In some embodiments, the proteinaceous molecule is other than a proteinaceous molecule comprising or consisting of the amino acid sequence of SEQ ID NO: 43 or 44 or a cyclized proteinaceous molecule thereof:

| [SEQ ID NO: 43] | |
|---|---|
| | DGGVCPKILKKCRRDSDCPGACICRGNGYCGSGS; |
| [SEQ ID NO: 44] | |
| | GGVCPKILKKCRRDSDCPGACICRGNGYCGSGSD. |

In some embodiments, X₁ is selected from P and modified forms thereof; and C and modified forms thereof. In some embodiments, X₁ is P or C, especially P. In some embodiments, X₁ is P or a modified form thereof, especially P.

In some embodiments, X₂ is K, R, H, S, T, A or G. In particular embodiments, X₂ is R, G or K; especially R or G; most especially R. In some embodiments, X₂ is selected from basic amino acid residues including K, R, H and modified forms thereof, especially K, R or modified forms thereof; most especially R.

In some embodiments, X₃ is S, T, A, G, F, Y, W, 4-F-Phe, 4-Me-Phe, V, L, I or Ne. In some embodiments, X₃ is I, L, V, F, G, Nle, 4-F-Phe or 4-Me-Phe; especially I, F, 4-F-Phe or 4-Me-Phe; more especially I or 4-F-Phe; most especially I.

In particular embodiments, X₃ is selected from aromatic amino acid residues including F, Y, W, 4-F-Phe, 4-Me-Phe and modified forms thereof; and hydrophobic amino acid residues including V, L, I, Ne and modified forms thereof. In some embodiments, X₃ is F, Y, W, 4-F-Phe, 4-Me-Phe, V, L, I or Nle; especially I, L, V, F, Nle, 4-F-Phe or 4-Me-Phe; more especially I, F, 4-F-Phe or 4-Me-Phe; more especially I or 4-F-Phe; most especially I.

In some embodiments, X₄ is S, T, A, G, F, Y, W, V, L, I, Nle, D, E or hGlu. In particular embodiments, X₄ is G, L, E, Y, V, W or Nle; especially, G, L, E or Nle; most especially G or E.

In particular embodiments, X₄ is selected from small amino acid residues including S, T, A, G and modified forms thereof; hydrophobic amino acid residues including V, L, I, Ne and modified forms thereof; and acidic amino acid residues including D, E, hGlu and modified forms thereof; especially S, T, A, G, V, L, I, Nle, D, E or hGlu; most especially G, L, E or Nle; more especially G or E.

In some embodiments, X₅ is S, T, A, G, F, Y, W, V, L, I, M, Nle, K, R, H, N or Q; especially R, K, V, W or L; most especially R or K.

In some embodiments, X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; and basic amino acid residues including K, R, H and modified forms thereof. In some embodiments, X₅ is S, T, A, G, F, Y, W, V, L, I, Nle, K, R or H; especially R, K, V, W or L; most especially R or K.

In some embodiments, X₅ is selected from aromatic amino acid residues including F, Y, W and modified forms thereof; hydrophobic amino acid residues including V, L, I, M, Ne and modified forms thereof; and basic amino acid residues including K, R, H and modified forms thereof; especially F, Y, W, V, L, I, M, Nle, K, R or H; more especially R, K, V, W or L; most especially R or K.

In some embodiments, X₆ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof. In some embodiments, X₆ is S, T, A, G, F, Y, W, V, L, I, Nle, K, R or H; especially K, L, Y, W, R, A or V; especially L, A or K. In particular embodiments, X₆ is L or K; especially L.

In some embodiments, X₇ is K, R, H, N, Q, S, T, A, G, D, E, V, L, I or Nle; especially is K, R, H, N, Q, S, T, A, G, E or V. In particular embodiments, X₇ is K, R, H, N, Q, S, T, A or G; especially K, R, H, N or Q; more especially K or R; most especially R.

In some embodiments, X₇ is selected from basic amino acid residues including K, R, H and modified forms thereof; and amide containing amino acid residues including N, Q and modified forms thereof; especially K, R, H, N or Q.

In some embodiments, X₇ is selected from basic amino acid residues including K, R, H and modified forms thereof; especially K, R or H; more especially K or R; most especially R.

In alternative embodiments, X₇ is selected from acidic amino acid residues including D, E and modified forms thereof (e.g. E); and hydrophobic amino acid residues including V, L, I, Ne and modified forms thereof (e.g. V). In some embodiments, X₇ is selected from D, E, V, L, I and Nle; especially E or V.

In some embodiments, X₈ is K, R, H, N or Q; especially K or R; most especially R.

In some embodiments, X₈ is selected from basic amino acid residues including K, R, H and modified forms thereof; especially K, R or H; more especially K or R; most especially R.

In some embodiments, X₉ is S, T, A, G, F, Y, W, V, L, I, Nle, K, R or H. In particular embodiments, X₉ is R, I, A, Y or V; especially R.

In particular embodiments, X₉ is selected from basic amino acid residues including K, R, H and modified forms thereof; especially K, R or H; most especially R.

In some embodiments, X₁₀ is P, S, T, A, G, F, Y, W, K, R or H; especially G, A, R, P or F.

In particular embodiments, X₁₀ is selected from small amino acid residues including S, T, A, G and modified forms thereof; especially S, T, A or G; most especially A or G.

In some embodiments, X₁₁ is N, Q, S, T, A, G, K, R or H; especially N, T, R, G or K; most especially N or T.

In some embodiments, X₁₂ is S, T, A, G, K, R, H, N or Q; especially G, R, T or K; most especially G or R.

In some embodiments, X₁₂ is selected from small amino acid residues including S, T, A, G and modified forms thereof; and basic amino acid residues including K, R, H and modified forms thereof; especially S, T, A, G, K, R or H; more especially G, R, T or K; most especially G or R.

In some embodiments, X₁₃ is K, R, H, F, Y, W, V, L, I or Nle; especially Y, F, L, W or H; most especially Y or F.

In particular embodiments:
X₁ is P;
X₂ is R;
X₃ is I, F, L, V or 4-F-Phe;
X₄ is L, E, Nle, V, W or G;
X₅ is K, R, V or W;
X₆ is K, L, Y, W, R or A;
X₇ is R or K;
X₈ is R or K;
X₉ is R, I, A or Y;
X₁₀ is G, A, R or P;
X₁₁ is N, T, R or G;
X₁₂ is R, T, G or K; and/or
X₁₃ is Y, F, L or W.

In some embodiments:
X₁ is P;
X₂ is R;
X₃ is I, F, or 4-F-Phe;
X₄ is L, E, Nle, V, W or G;
X₅ is K or R;
X₆ is K, L or A;
X₇ is R or K;
X₅ is R or K;
X₉ is R;
X₁₀ is G or A;
X₁₁ is N or T;
X₁₂ is R or G; and/or
X₁₃ is Y or F.

In particular embodiments:
X₁ is selected from P and modified forms thereof;
X₂ is selected from basic amino acid residues including K, R, H and modified forms thereof;
X₃ is selected from hydrophobic amino acid residues including V, L, I, Ne and modified forms thereof;
X₄ is selected from small amino acid residues including S, T, A, G and modified forms thereof;
X₅ is selected from basic amino acid residues including K, R, H and modified forms thereof;
X₆ is selected from hydrophobic amino acid residues including V, L, I, Ne and modified forms thereof;
X₇ is selected from basic amino acid residues including K, R, H and modified forms thereof; and amide containing amino acid residues including N, Q and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof.

In further embodiments:
X₁ is selected from P and modified forms thereof;
X₂ is selected from basic amino acid residues including K, R, H and modified forms thereof;
X₃ is selected from hydrophobic amino acid residues including V, L, I, Ne and modified forms thereof;
X₄ is selected from small amino acid residues including S, T, A, G and modified forms thereof;
X₅ is selected from basic amino acid residues including K, R, H and modified forms thereof;
X₆ is selected from hydrophobic amino acid residues including V, L, I, Ne and modified forms thereof;
X₇ is selected from basic amino acid residues including K, R, H and modified forms thereof;
X₈ is selected from basic amino acid residues including K, R, H and modified forms thereof;
X₉ is selected from basic amino acid residues including K, R, H and modified forms thereof;
X₁₀ is selected from small amino acid residues including S, T, A, G and modified forms thereof;
X₁₁ is selected from small amino acid residues including S, T, A, G and modified forms thereof;
X₁₂ is selected from basic amino acid residues including K, R, H and modified forms thereof; and X₁₃ is selected from aromatic amino acid residues including F, Y, W and modified forms thereof.

In specific embodiments:
X₁ is P;
X₂ is K, R or H; especially K or R; most especially R;
X₃ is V, L, I or Nle; preferably I;
X₄ is S, T, A or G; preferably G;
X₅ is K, R or H; especially K or R; most especially R;
X₆ is selected from V, L, I and Nle; especially L;
X₇ is K, R or H; especially K or R; most especially R;
X₈ is K, R or H; especially K or R; most especially R;
X₉ is K, R or H; especially K or R; most especially R;
X₁₀ is S, T, A or G; especially A;
X₁₁ is S, T, A or G; especially T;
X₁₂ is K, R, H; especially K or R; most especially R; and/or
X₁₃ is F, Y or W; especially F.

In alternative embodiments:
X₁ is selected from P and modified forms thereof; and C and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; and hydrophobic amino acid residues including V, L, I, Ne and modified forms thereof;
X₅ is selected from aromatic amino acid residues including F, Y, W and modified forms thereof; hydrophobic amino acid residues including V, L, I, M, Ne and modified forms thereof; and basic amino acid residues including K, R, H and modified forms thereof;
X₆ is selected from aromatic amino acid residues including F, Y, W and modified forms thereof; hydrophobic amino acid residues including V, L, I, Ne and modified forms thereof; and basic amino acid residues including K, R, H and modified forms thereof;
X₇ is selected from basic amino acid residues including K, R, H and modified forms thereof; and amide containing amino acid residues including N, Q and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof.

In particular embodiments:
X₁ is P or C;
X₂ is K, R, H, S, T, A or G; especially R or G;
X₃ is S, T, A, G, V, L, I or Nle; especially I, L, V or G;
X₄ is S, T, A, G, F, Y, W, V, L, I or Nle; especially G, L or Y;
X₅ is F, Y, W, V, L, I, M, Nle, K, R or H; especially R, V, W or L;
X₆ is F, Y, W, V, L, I, Nle, K, R or H; especially L, Y, W, R or V;
X₇ is K, R, H, N or Q; especially K or R; most especially R;
X₈ is K, R, H, N or Q; especially K or R; most especially R;
X₉ is S, T, A, G, F, Y, W, V, L, I, Nle, K, R or H; especially R, I, A, Y or V;
X₁₀ is P, S, T, A, G, F, Y, W, K, R or H; especially A, R, P or F;
X₁₁ is N, Q, S, T, A, G, K, R or H; especially T, R, G or K;
X₁₂ is S, T, A, G, K, R, H, N or Q; especially T, R, G or K; and/or
X₁₃ is K, R, H, F, Y, W, V, L, I or Nle; especially F, Y, L, W or H.

In some embodiments, the proteinaceous molecule comprises, consists or consists essentially of an amino acid sequence represented by any one of SEQ ID NOs: 8 to 36:

[SEQ ID NO: 8]

DGGICPRIGRLCRRDSDCPGACICRATRFCGSGY;

[SEQ ID NO: 9]

GGICPRIGRLCRRDSDCPGACICRATRFCGSGYD;

[SEQ ID NO: 10]

GGICPRIGRLCRRDSDCPGACICRATRFCGSGSD;

[SEQ ID NO: 11]

DGGICPRILVYCRRDSDCPGACICIRRTYCGSGS;

[SEQ ID NO: 12]

GGICPRILVYCRRDSDCPGACICIRRTYCGSGSD;

[SEQ ID NO: 13]

DGGRCPRLLRWCRRDSDCPGACICARGGLCGSGS;

[SEQ ID NO: 14]

GGRCPRLLRWCRRDSDCPGACICARGGLCGSGSD;

[SEQ ID NO: 15]

DGGVCPRVGWRCRRDSDCPGACICYPTKWCGSGS;

[SEQ ID NO: 16]

GGVCPRVGWRCRRDSDCPGACICYPTKWCGSGSD;

[SEQ ID NO: 17]

DGGRCCGGYLVCRRDSDCPGACICVFKKHCGSGS;

[SEQ ID NO: 18]

GGRCCGGYLVCRRDSDCPGACICVFKKHCGSGSD;

[SEQ ID NO: 19]

DGGICPRIGRLCRRDSDCPGACICRGNGYCGSGS;

[SEQ ID NO: 20]

GGICPRIGRLCRRDSDCPGACICRGNGYCGSGSD;

[SEQ ID NO: 21]

DGGVCPKILKKCRRDSDCPGACICRATRFCGSGS;

[SEQ ID NO: 22]

GGVCPKILKKCRRDSDCPGACICRATRFCGSGSD;

[SEQ ID NO: 23]

RICPRIGRLCRRDSDCPGACICRATRFCG;

[SEQ ID NO: 24]

GGICPRIGRLCKRDSDCPGACICRATRFCGSGSD;

[SEQ ID NO: 25]

GGICPRIGRLCRKDSDCPGACICRATRFCGSGSD;

[SEQ ID NO: 26]

GGICPRIGRLCRRDSDCPGACICRATRFCGSGKD;

[SEQ ID NO: 27]

GGICPRFGRLCRRDSDCPGACICRATRFCGSGSD;

[SEQ ID NO: 28]

GGRCPRIGRLCRRDSDCPGACICRATRFCGSGSD;

[SEQ ID NO: 29]

RVCPR[4-F-Phe]EKKCRRDSDCPGACICRGNGYCG;

[SEQ ID NO: 30]

RVCPR[4-F-Phe]VKKCRRDSDCPGACICRGNGYCG;

[SEQ ID NO: 31]

RVCPR[4-F-Phe]WKKCRRDSDCPGACICRGNGYCG;

[SEQ ID NO: 32]

RVCPR[4-F-Phe]ERKCRRDSDCPGACICRGNGYCG;

[SEQ ID NO: 33]

RVCPR[4-F-Phe]VRKCRRDSDCPGACICRGNGYCG;

[SEQ ID NO: 34]

RVCPR[4-F-Phe]WRKCRRDSDCPGACICRGNGYCG;

[SEQ ID NO: 35]

RVCPR[4-F-Phe][Nle]KACRRDSDCPGACICRGNGYCG;
or

[SEQ ID NO: 36]

GGVCPR[4-F-Phe]EKKCRRDSDCPGACICRGNGYCGSGSD.

In some embodiments, the proteinaceous molecule comprises, consists or consists essentially of an amino acid sequence represented by any one of SEQ ID NOs: 8 to 23, especially any one of SEQ ID NOs: 8 to 22, most especially any one of SEQ ID NOs: 8 to 10, 19 and 20. In particular embodiments, the proteinaceous molecule comprises, consists or consists essentially of an amino acid sequence represented by SEQ ID NO: 8 or 19. In some embodiments, the proteinaceous molecule comprises, consists or consists essentially of an amino acid sequence represented by SEQ ID NO: 10. In some embodiments, the proteinaceous molecule comprises, consists or consists essentially of an amino acid sequence represented by SEQ ID NO: 25.

In some embodiments, the proteinaceous molecule comprises, consists or consists essentially of an amino acid sequence represented by any one of SEQ ID NOs: 45 to 50:

[SEQ ID NO: 45]
GGICPRIGRLCQRDSDCPGACICRATRFCGSGSD;
[SEQ ID NO: 46]
GGICPRIGRLCRQDSDCPGACICRATRFCGSGSD;
[SEQ ID NO: 47]
GGICPRIGRLCRRDSDCPGACICRATQFCGSGSD;
[SEQ ID NO: 48]
GGICPRIGRLCQQDSDCPGACICRATRFCGSGSD;
[SEQ ID NO: 49]
GGICPRIGRLCQRDSDCPGACICRATQFCGSGSD;
and
[SEQ ID NO: 50]
GGICPRIGRLCRQDSDCPGACICRATQFCGSGSD.

In some embodiments, the proteinaceous molecule comprises, consists or consists essentially of an amino acid sequence represented by any one of SEQ ID NOs: 149-156:

[SEQ ID NO: 149]
GGICPRIGRLCRRDSDCPGACICRPTRFCGSGSD;
[SEQ ID NO: 150]
GGICPRIGRLCRRDSDCPGACICRKTRFCGSGSD;
[SEQ ID NO: 151]
GGICPRIGRLCRRDSDCPGACICRATGFCGSGSD;
[SEQ ID NO: 152]
GGICPRIGRLCRRDSDCPGACICRATHFCGSGSD;
[SEQ ID NO: 153]
GGICPRIGRLCVRDSDCPGACICRATRFCGSGSD;
[SEQ ID NO: 154]
GGICPRIGRLCERDSDCPGACICRATRFCGSGSD;
[SEQ ID NO: 155]
GGICPRIGRLCTRDSDCPGACICRATRFCGSGSD;
and
[SEQ ID NO: 156]
GGICPRIGRLCRRDSDCPGACICRATRFCGSGSP.

In some embodiments, the proteinaceous molecule comprises, consists or consists essentially of an amino acid sequence represented by SEQ ID NO: 150, 153 or 155.

In some embodiments, the proteinaceous molecule is a proteinaceous molecule comprising, consisting or consisting essentially of an amino acid sequence represented by Formula II:

X₁₄X₁₅X₁₆X₁₇CX₁X₂X₃X₄X₅X₆CX₇X₈DSDCPGACICX₉X₁₀X₁₁X₁₂X₃CX₁₈X₁₉X₂₀X₂₁X₂₂ (II)

wherein:
X₁ to X₁₃ are as defined for Formula I; and
X₁₄ to X₂₂ are independently absent or are selected from any amino acid residue.

In some embodiments, the proteinaceous molecule is other than a proteinaceous molecule comprising or consisting of the amino acid sequence of any one of SEQ ID NOs: 1 to 7.

In some embodiments, when X₁ is P, X₂ is K, X₃ is I, X₄ is L, X₅ is K and X₆ is K; X₉ is other than R, X₁₀ is other than G, X₁₁ is other than N, X₁₂ is other than G and/or X₁₃ is other than Y. In alternative embodiments, the proteinaceous molecule does not comprise an amino acid sequence of any one of SEQ ID NOs: 37 to 42.

In some embodiments, the proteinaceous molecule is other than a proteinaceous molecule comprising or consisting of the amino acid sequence of SEQ ID NO: 43 or 44, or a cyclized proteinaceous molecule thereof.

In particular embodiments, when X₁₄ is present, X₁₅, X₁₆ and X₁₇ are present; when X₁₅ is present, X₁₆ and X₁₇ are present; and when X₁₆ is present, X₁₇ is present. Accordingly, when X₁₇ is absent, X₁₄, X₁₅ and X₁₆ are absent; when X₁₆ is absent, X₁₄ and X₁₅ are absent; and when X₁₅ is absent, X₁₄ is absent.

In particular embodiments, when X₂₂ is present, X₁₈, X₁₉, X₂₀ and X₂₁ are present; when X₂₁ is present, X₁₈, X₁₉ and X₂₀ are present; when X₂₀ is present, X₁₈ and X₁₉ are present; and when X₁₉ is present, X₁₈ is present. Accordingly, when X₁₈ is absent, X₁₉, X₂₀, X₂₁ and X₂₂ are absent; when X₁₉ is absent, X₂₀, X₂₁ and X₂₂ are absent; when X₂₀ is absent, X₂₁ and X₂₂ are absent; and when X₂₁ is absent, X₂₂ is absent.

In some embodiments, X₁₄ is absent or is selected from acidic amino acid residues including D, E and modified forms thereof; especially absent or is D or E; most especially absent or is D.

In some embodiments, X₁₄ is absent or is selected from acidic amino acid residues including D, E and modified forms thereof, and P and modified forms thereof; especially absent or is D, E or P; most especially absent or is D.

In some embodiments, X₁₅ is absent or is selected from small amino acid residues including S, T, A, G and modified forms thereof; especially absent, or is S, T, A or G; most especially absent or is G.

In some embodiments, X₁₆ is absent or is selected from small amino acid residues including S, T, A, G and modified forms thereof; and basic amino acid residues including R, K, H and modified forms thereof; especially absent or is S, T, A, G, R, K or H; more especially absent or is G or R; most especially G or R.

In some embodiments, X₁₇ is absent or is selected from hydrophobic amino acid residues including V, L, I, Ne and modified forms thereof; and basic amino acid residues including K, R, H and modified forms thereof. In particular embodiments, X₁₇ is absent or is V, L, I, Nle, K, R or H; especially absent or is V, I or R; most especially V, I or R.

In particular embodiments,
X₁₄ is absent or is D;
X₁₅ is absent or is G;
X₁₆ is absent or is G or R; and/or
X₁₇ is absent or is V, I or R.

In some embodiments, X₁₄ is D; X₁₅ is G; X₁₆ is G; and/or X₁₇ is V, I or R.

In alternative embodiments, X₁₄ is absent; X₁₅ is G; X₁₆ is G or R; and/or X₁₇ is V, I or R.

In alternative embodiments, X₁₄ is absent; X₁₅ is absent; X₁₆ is R; and/or X₁₇ is V or I.

In some embodiments, X₁₈ is absent or is selected from small amino acid residues including S, T, A, G and modified forms thereof; especially absent or is S, T, A or G; most especially absent or is G.

In some embodiments, X₁₉ is absent or is selected from small amino acid residues including S, T, A, G and modified forms thereof; especially absent or is S, T, A or G; most especially absent or is S.

In some embodiments, X₂₀ is absent or is selected from small amino acid residues including S, T, A, G and modified forms thereof; especially absent or is S, T, A or G; most especially absent or is G.

In some embodiments, X₂₁ is absent or is selected from small amino acid residues including S, T, A, G and modified forms thereof; aromatic amino acid residues including F, Y, W and modified forms thereof; and basic amino acid residues including K, R, H and modified forms thereof. In particular embodiments, X₂₁ is absent or is S, T, A, G, F, Y, W, K, R or H; especially absent or is S, Y or K; especially absent or is S.

In some embodiments, X₂₂ is absent or is selected from acidic amino acid residues including D, E and modified forms thereof; especially absent or is D or E; more especially absent or is D.

In some embodiments, X₂₂ is absent or is selected from acidic amino acid residues including D, E and modified forms thereof, and P and modified forms thereof; especially absent or is D, E or P; most especially absent or is D.

In some embodiments,
X₁₈ is absent or is G;
X₁₉ is absent or is S;
X₂₀ is absent or is G;
X₂₁ is absent or is S, Y or K; and/or
X₂₂ is absent or is D.

In some embodiments, X₁₈ is G and X₁₉ to X₂₂ are absent.

In alternative embodiments, X₁₈ is G; X₁₉ is S; X₂₀ is G; X₂₁ is S, Y or K; and/or X₂₂ is absent.

In alternative embodiments, X₁₈ is G; X₁₉ is S; X₂₀ is G; X₂₁ is S, Y or K; and/or X₂₂ is D.

In alternative embodiments, X₁₈ is G; X₁₉ is S; X₂₀ is G; X₂₁ is S or K; and/or X₂₂ is D.

Suitable embodiments of each of X₁ to X₁₃ are as discussed supra for Formula I.

In some embodiments, the proteinaceous molecule comprises, consists or consists essentially of an amino acid sequence represented by any one of SEQ ID NOs: 8 to 36, 45 to 50 and 149 to 156; especially any one of SEQ ID NOs: 8 to 36 and 45 to 50; more especially any one of SEQ ID NOs: 8 to 36; most especially any one of SEQ ID NOs: 8 to 23. In some embodiments, the proteinaceous molecule comprises, consists or consists essentially of an amino acid sequence represented by any one of SEQ ID NOs: 8 to 10, 19 and 20; especially 8 or 19.

In some embodiments, the proteinaceous molecule is a proteinaceous molecule comprising, consisting or consisting essentially of an amino acid sequence represented by Formula III:

X₁₆X₁₇CX₁X₂X₃X₄X₅X₆CX₇X₈DSDCPGACICX₉X₁₀X₁₁X₁₂X₁₃CX₁₈ (III)

wherein:
X₁ to X₁₃ are as defined for Formula I; and
X₁₆ to X₁₈ are as defined for Formula II.

In some embodiments, the proteinaceous molecule is other than a proteinaceous molecule comprising or consisting of the amino acid sequence of any one of SEQ ID NOs: 1 to 7.

In some embodiments, when X₁ is P, X₂ is K, X₃ is I, X₄ is L, X₅ is K and X₆ is K; X₉ is other than R, X₁₀ is other than G, X₁₁ is other than N, X₁₂ is other than G and/or X₁₃ is other than Y. In alternative embodiments, the proteinaceous molecule does not comprise an amino acid sequence of any one of SEQ ID NOs: 37 to 42.

In some embodiments, the proteinaceous molecule is other than a proteinaceous molecule comprising or consisting of the amino acid sequence of SEQ ID NO: 43 or 44 or a cyclized proteinaceous molecule thereof.

Suitable embodiments of each of X₁ to X₁₃ and X₁₆ to X₁₈ are as discussed supra for Formula I and Formula II.

In some embodiments, the proteinaceous molecule comprises, consists or consists essentially of an amino acid sequence represented by any one of SEQ ID NOs: 8 to 36, 45 to 50 and 149 to 156; especially any one of SEQ ID NOs: 8 to 36 and 45 to 50; more especially any one of SEQ ID NOs: 8 to 36; most especially any one of SEQ ID NOs: 8 to 23. In some embodiments, the proteinaceous molecule comprises, consists or consists essentially of an amino acid sequence represented by any one of SEQ ID NOs: 8 to 10, 19 and 20; especially 8 or 19.

In some embodiments, the proteinaceous molecule is a proteinaceous molecule comprising, consisting or consisting essentially of an amino acid sequence represented by Formula IV:

Z₁CX₁X₂X₃X₄X₅X₆CX₇X₈DSDCPGACICX₉X₁₀X₁₁X₁₂X₁₃CZ₂ (IV)

wherein:
X₁ to X₁₃ are as defined for Formula I; and
Z₁ and Z₂ are independently absent or are independently selected from at least one of a proteinaceous moiety consisting of from about 1 to about 50 amino acid residues (and all integer residues in between), and a protecting moiety.

In some embodiments, the proteinaceous molecule is other than a proteinaceous molecule comprising or consisting of the amino acid sequence of any one of SEQ ID NOs: 1 to 7.

In some embodiments, when X₁ is P, X₂ is K, X₃ is I, X₄ is L, X₅ is K and X₆ is K; X₉ is other than R, X₁₀ is other than G, X₁₁ is other than N, X₁₂ is other than G and/or X₁₃ is other than Y. In alternative embodiments, the proteinaceous molecule does not comprise an amino acid sequence of any one of SEQ ID NOs: 37 to 42.

In some embodiments, the proteinaceous molecule is other than a proteinaceous molecule comprising or consisting of the amino acid sequence of SEQ ID NO: 43 or 44 or a cyclized proteinaceous molecule thereof.

In some embodiments, Z₁ is absent or is a proteinaceous moiety consisting of from about 1 to about 10 amino acid residues (and all integer residues in between); especially about 2 to about 4 amino acid residues (and all integer residues in between). The amino acid residues are selected from any amino acid residues.

In some embodiments, Z₂ is absent or is a proteinaceous moiety consisting of from about 1 to about 10 amino acid residues (and all integer residues in between); especially about 1 to about 5 amino acid residues (and all integer residues in between). The amino acid residues are selected from any amino acid residues.

Suitable embodiments of X₁ to X₁₃ are as discussed supra for Formula I.

In some embodiments, the proteinaceous molecule comprises, consists or consists essentially of an amino acid sequence represented by any one of SEQ ID NOs: 8 to 36, 45 to 50 and 149 to 156; especially any one of SEQ ID NOs: 8 to 36 and 45 to 50; more especially any one of SEQ ID NOs: 8 to 36; most especially any one of SEQ ID NOs: 8 to 23. In some embodiments, the proteinaceous molecule comprises, consists or consists essentially of an amino acid sequence represented by any one of SEQ ID NOs: 8 to 10, 19 and 20; especially 8 or 19.

In another aspect, there is provided a proteinaceous molecule comprising an amino acid sequence represented by Formula V:

CX₁X₂X₃X₄X₅X₆CX₇X₈DSDCX₂₃X₂₄X₂₅CX₂₆CX₉X₁₀X₁X₁₂X₁₃C (V)

wherein:
X₁ to X₁₃ are as defined for Formula I;
X₂₃ is selected from P and modified forms thereof; and hydrophobic amino acid residues including V, L, I, M, Ne and modified forms thereof;
X₂₄ is selected from small amino acid residues including S, T, A, G and modified forms thereof;
X₂₅ is selected from small amino acid residues including S, T, A, G and modified forms thereof; basic amino acid residues including K, R, H and modified forms thereof; amide containing amino acid residues including N, Q and modified forms thereof; and acidic amino acid residues including D, E, hGlu and modified forms thereof; and
X₂₆ is selected from hydrophobic amino acid residues including V, L, I, M, Ne and modified forms thereof; small amino acid residues including S, T, A, G and modified forms thereof; and basic amino acid residues including K, R, H and modified forms thereof.

In some embodiments, the proteinaceous molecule is other than a proteinaceous molecule comprising or consisting of the amino acid sequence of any one of SEQ ID NOs: 1 to 7.

In some embodiments, when X₁ is P, X₂ is K, X₃ is I, X₄ is L, X₅ is K and X₆ is K; X₉ is other than R, X₁₀ is other than G, X₁₁ is other than N, X₁₂ is other than G and/or X₁₃ is other than Y. In alternative embodiments, the proteinaceous molecule does not comprise an amino acid sequence of any one of SEQ ID NOs: 37 to 42.

In some embodiments, the proteinaceous molecule is other than a proteinaceous molecule comprising or consisting of the amino acid sequence of any one of SEQ ID NOs: 51-53:

[SEQ ID NO: 51]
CPRILKKCRRDSDCPGACVCKGNGYC;
[SEQ ID NO: 52]
CPKILQRCRRDSDCPSACICRGNGYC;
or
[SEQ ID NO: 53]
CPRILKKCRRDSDCPGACVCRGNGYC.

In some embodiments, the proteinaceous molecule is other than a proteinaceous molecule comprising or consisting of the amino acid sequence of SEQ ID NO: 43 or 44 or a cyclized proteinaceous molecule thereof.

Suitable embodiments of each of X₁ to X₁₃ are as discussed supra for Formula I.

In some embodiments:
X₂₃ is P, L or M;
X₂₄ is G, S or A;
X₂₅ is A, E, Q or K; and/or
X₂₆ is I, V, K, R or T; especially I or K.

In some embodiments, the proteinaceous molecule comprises, consists or consists essentially of an amino acid sequence represented by any one of SEQ ID NOs: 8 to 36, 45 to 50, 54 and 149-156; especially any one of SEQ ID NOs: 8 to 36, 45 to 50 and 54:

[SEQ ID NO: 54]
GGICPRIGRLCRRDSDCPGACKCRATRFCGSGSD.

In some embodiments, the proteinaceous molecule is a proteinaceous molecule comprising, consisting or consisting essentially of an amino acid sequence represented by Formula VI:

X₁₄X₁₅X₁₆X₁₇CX₁X₂X₃X₄X₅X₆CX₇X₈DSDCX₂₃X₂₄X₂₅CX₂₆CX₉X₁₀X₁₁X₁₂X₁₃CX₁₈X₁₉X₂₀X₂₁X₂₂ (VI)

wherein:
X₁ to X₁₃ are as defined for Formula I; and
X₁₄ to X₂₂ are as defined for Formula II; and
X₂₃ to X₂₆ are as defined for Formula V.

Suitable embodiments of each of X₁ to X₂₆ are as discussed supra for Formulae I, II and V.

In some embodiments, X₁₄, X₁₅ and X₁₉ to X₂₂ are absent.

In some embodiments, the proteinaceous molecule is other than a proteinaceous molecule comprising or consisting of the amino acid sequence of any one of SEQ ID NOs: 1 to 7. In some embodiments, the proteinaceous molecule is other than a proteinaceous molecule comprising or consisting of the amino acid sequence of any one of SEQ ID NOs: 51-53.

In some embodiments, when X₁ is P, X₂ is K, X₃ is I, X₄ is L, X₅ is K and X₆ is K; X₉ is other than R, X₁₀ is other than G, X₁₁ is other than N, X₁₂ is other than G and/or X₁₃ is other than Y. In alternative embodiments, the proteinaceous molecule does not comprise an amino acid sequence of any one of SEQ ID NOs: 37 to 42.

In some embodiments, the proteinaceous molecule is other than a proteinaceous molecule comprising or consisting of the amino acid sequence of SEQ ID NO: 43 or 44 or a cyclized proteinaceous molecule thereof.

In another aspect, there is provided a proteinaceous molecule comprising, consisting or consisting essentially of an amino acid sequence represented by Formula VII:

CPRIGRX₆CX₇X₈X₂₇X₂₈X₂₉CX₂₃GACX₂₆CRX₁₀TX₁₂FC (VII)

wherein:
X₆ is selected from L, V, T, I and modified forms of any of the foregoing amino acids;
X₇ is selected from R, W, V, T, S, Q, N, M, Nle, L, K, I, F, E, D, A and modified forms of any of the foregoing amino acids;
X₈ is selected from R, Y, V, T, Q, M, Nle, L, K, I, H, F, E, A and modified forms of any of the foregoing amino acids;
X₂₇ is selected from D, T, N, H and modified forms of any of the foregoing amino acids;
X₂₈ is selected from S, T, A and modified forms of any of the foregoing amino acids;
X₂₉ is selected from D, E and modified forms of any of the foregoing amino acids;
X₂₃ is selected from P, Y, M, Nle, L, I, F and modified forms of any of the foregoing amino acids;
X₂₆ is selected from I, V, K and modified forms of any of the foregoing amino acids;
X₁₀ is selected from A, V, T, S, R, P, K and modified forms of any of the foregoing amino acids; and
X₁₂ is selected from R, K, H, G and modified forms of any of the foregoing amino acids.

In some embodiments, X₆ is selected from L, V, T and I; especially T or I.

In some embodiments, X₇ is selected from R, W, V, T, S, Q, N, M, Nle, L, K, I, F, E, D and A; especially R, W, V, T, S, Q, N, M, L, K, I, F, E, D or A. In particular embodiments, X₇ is selected from R, W, V, T, S, Q, N, M, L, K, I, E and D; especially V, T or I.

In some embodiments, X₈ is selected from R, Y, V, T, Q, M, Nle, L, K, I, H, F, E and A; especially R, Y, V, T, Q, M, L, K, I, H, F, E or A. In particular embodiments, X₈ is Y, V or Q.

In some embodiments, X₂₇ is selected from D, T, N and H; especially D.

In some embodiments, X₂₈ is selected from S, T and A; especially S.

In some embodiments, X₂₉ is selected from D and E; especially E.

In some embodiments, X₂₃ is selected from P, Y, M, Nle, L, I and F; especially P, Y, M, L, I or F. In particular embodiments, X₂₃ is selected from Y, M, L and F; especially L.

In some embodiments, X₂₆ is selected from I, V and K; especially I and V; more especially I. In some embodiments, X₂₆ is selected from I and V and modified forms of any of the foregoing amino acids; especially I and V; most especially I.

In some embodiments, X₁₀ is selected from A, V, T, S, R, P and K; especially R, P or K.

In some embodiments, X₁₂ is selected from R, K, H and G; especially H or G; more especially G.

In some embodiments:
X₆ is selected from L, V, T and I;
X₇ is selected from R, W, V, T, S, Q, N, M, Nle, L, K, I, F, E, D and A;
X₈ is selected from R, Y, V, T, Q, M, Nle, L, K, I, H, F, E and A;
X₂₇ is selected from D, T, N and H;
X₂₈ is selected from S, T and A;
X₂₉ is selected from D and E;
X₂₃ is selected from P, Y, M, Nle, L, I and F;
X₂₆ is selected from I, V and K; especially I or V;
X₁₀ is selected from A, V, T, S, R, P and K; and/or
X₁₂ is selected from R, K, H and G.

In some embodiments:
X₆ is selected from L, V, T and I;
X₇ is selected from R, W, V, T, S, Q, N, M, L, K, I, F, E, D and A;
X₈ is selected from R, Y, V, T, Q, M, L, K, I, H, F, E and A;
X₂₇ is selected from D, T, N and H;
X₂₈ is selected from S, T and A;
X₂₉ is selected from D and E;
X₂₃ is selected from P, Y, M, L, I and F;
X₂₆ is selected from I, V and K; especially I or V;
X₁₀ is selected from A, V, T, S, R, P and K; and/or
X₁₂ is selected from R, K, H and G.

In some embodiments:
X₆ is T or I;
X₇ is selected from R, W, V, T, S, Q, N, M, L, K, I, E and D; especially V, T or I;
X₈ is Y, V or Q;
X₂₇ is D;
X₂₈ is S;
X₂₉ is E;
X₂₃ is selected from Y, M, L and F; especially L;
X₂₆ is I;
X₁₀ is R, P or K; and/or
X₁₂ is H or G; especially G.

In some embodiments, the proteinaceous molecule is a proteinaceous molecule comprising, consisting or consisting essentially of an amino acid sequence represented by Formula VIII:

X₁₄X₁₅X₁₆X₁₇CPRIGRX₆CX₇X₈X₂₇X₂₈X₂₉CX₂₃GACX₂₆CRX₁₀TX₁₂FCX₁₈X₁₉X₂₀X₂₁X₂₂ (VIII)

wherein:
X₆, X₇, X₈, X₂₇, X₂₈, X₂₉, X₂₃, X₂₆, X₁₀ and X₁₂ are as defined for Formula VII; and
X₁₄ to X₂₂ are independently absent or are selected from any amino acid residue.

In particular embodiments, when X₁₄ is present, X₁₅, X₁₆ and X₁₇ are present; when X₁₅ is present, X₁₆ and X₁₇ are present; and when X₁₆ is present, X₁₇ is present. Accordingly, when X₁₇ is absent, X₁₄, X₁₅ and X₁₆ are absent; when X₁₆ is absent, X₁₄ and X₁₅ are absent; and when X₁₅ is absent, X₁₄ is absent.

In particular embodiments, when X₂₂ is present, X₁₈, X₁₉, X₂₀ and X₂₁ are present; when X₂₁ is present, X₁₅, X₁₉ and X₂₀ are present; when X₂₀ is present, X₁₈ and X₁₉ are present; and when X₁₉ is present, X₁₈ is present. Accordingly, when X₁₈ is absent, X₁₉, X₂₀, X₂₁ and X₂₂ are absent; when X₁₉ is absent, X₂₀, X₂₁ and X₂₂ are absent; when X₂₀ is absent, X₂₁ and X₂₂ are absent; and when X₂₁ is absent, X₂₂ is absent.

In some embodiments, X₁₄ is selected from any amino acid residue. In some embodiments, X₁₄ is Y, W, V, T, S, R, Q, P, N, M, Nle, L, K, I, H, G, F, E, D, C, A or a modified form of any of the foregoing amino acids; especially Y, W, V, T, S, R, Q, P, N, M, Nle, L, K, I, H, G, F, E, D, C or A; more especially Y, W, V, T, S, R, Q, P, N, M, L, K, I, H, G, F, E, D, C or A; most especially R, K or H. In alternative embodiments, X₁₄ is absent.

In some embodiments, X₁₅ is selected from G, Y, T, S, R, K, H and modified forms of any of the foregoing amino acids; especially G, Y, T, S, R, K or H; more especially G, S, R, K or H.

In some embodiments, X₁₆ is selected from G, Y, R, K, H and modified forms of any of the foregoing amino acids; especially G, Y, R, K or H; more especially R, K or H.

In some embodiments, X₁₇ is I or a modified form thereof; especially I.

In some embodiments, X₁₈ is G or a modified form thereof; especially G.

In some embodiments, X₁₉ is selected from S, R, G and modified forms of any of the foregoing amino acids; especially S, R or G; more especially G.

In some embodiments, X₂₀ is selected from G, Y, W, V, S, R, Q, P, N, M, Nle, K, H, A and modified forms of any of the foregoing amino acids; especially G, Y, W, V, S, R, Q, P, N, M, Nle, K, H or A; more especially G, Y, W, V, S, R, Q, P, N, M, K, H or A. In particular embodiments, X₂₀ is R, Q, P, N, K, or A; especially R, P or K.

In some embodiments, X₂₁ is selected from S, Y, V, T, R, Q, P, N, M, Nle, L, K, I, H, G, F and modified forms of any of the foregoing amino acids; especially S, Y, V, T, R, Q, P, N, M, Nle, L, K, I, H, G or F; more especially S, Y, V, T, R, Q, P, N, M, L, K, I, H, G or F. In particular embodiments, X₂₁ is R, P, K, H, G or S; especially R, P, K, H or G.

In some embodiments, X₂₂ is selected from any amino acid residue. In some embodiments, X₂₂ is Y, W, V, T, S, R, Q, P, N, M, Nle, L, K, I, H, G, F, E, D, C, A or a modified form of any of the foregoing amino acids; especially Y, W, V, T, S, R, Q, P, N, M, Nle, L, K, I, H, G, F, E, D, C or A; more especially Y, W, V, T, S, R, Q, P, N, M, L, K, I, H, G, F, E, D, C or A; most especially R, K or H. In alternative embodiments, X₂₂ is absent.

In some embodiments:
X₁₄ is absent or is selected from Y, W, V, T, S, R, Q, P, N, M, Nle, L, K, I, H, G, F, E, D, C, A and modified forms of any of the foregoing amino acids;
X₁₅ is selected from G, Y, T, S, R, K, H and modified forms of any of the foregoing amino acids;
X₁₆ is selected from G, Y, R, K, H and modified forms of any of the foregoing amino acids;
X₁₇ is I or a modified form thereof;
X₁₈ is G or a modified form thereof;
X₁₉ is selected from S, R, G and modified forms of any of the foregoing amino acids;
X₂₀ is selected from G, Y, W, V, S, R, Q, P, N, M, Nle, K, H, A and modified forms of any of the foregoing amino acids;
X₂₁ is selected from S, Y, V, T, R, Q, P, N, M, Nle, L, K, I, H, G, F and modified forms of any of the foregoing amino acids; and
X₂₂ is absent or is selected from Y, W, V, T, S, R, Q, P, N, M, Nle, L, K, I, H, G, F, E, D, C, A and modified forms of any of the foregoing amino acids.

In some embodiments:
X₁₄ is selected from Y, W, V, T, S, R, Q, P, N, M, Nle, L, K, I, H, G, F, E, D, C, A and modified forms of any of the foregoing amino acids;
X₁₅ is selected from G, Y, T, S, R, K, H and modified forms of any of the foregoing amino acids;
X₁₆ is selected from G, Y, R, K, H and modified forms of any of the foregoing amino acids;
X₁₇ is I or a modified form thereof;
X₁₈ is G or a modified form thereof;
X₁₉ is selected from S, R, G and modified forms of any of the foregoing amino acids;
X₂₀ is selected from G, Y, W, V, S, R, Q, P, N, M, Nle, K, H, A and modified forms of any of the foregoing amino acids;
X₂₁ is selected from S, Y, V, T, R, Q, P, N, M, Nle, L, K, I, H, G, F and modified forms of any of the foregoing amino acids; and
X₂₂ is absent.

In some embodiments:
X₁₄ is absent;
X₁₅ is selected from G, Y, T, S, R, K, H and modified forms of any of the foregoing amino acids;
X₁₆ is selected from G, Y, R, K, H and modified forms of any of the foregoing amino acids;
X₁₇ is I or a modified form thereof;
X₁₈ is G or a modified form thereof;
X₁₉ is selected from S, R, G and modified forms of any of the foregoing amino acids;
X₂₀ is selected from G, Y, W, V, S, R, Q, P, N, M, Nle, K, H, A and modified forms of any of the foregoing amino acids;
X₂₁ is selected from S, Y, V, T, R, Q, P, N, M, Nle, L, K, I, H, G, F and modified forms of any of the foregoing amino acids; and
X₂₂ is selected from Y, W, V, T, S, R, Q, P, N, M, Nle, L, K, I, H, G, F, E, D, C, A and modified forms of any of the foregoing amino acids.

In particular embodiments:
X₁₄ is Y, W, V, T, S, R, Q, P, N, M, L, K, I, H, G, F, E, D, C or A; especially R, K or H;
X₁₅ is G, Y, T, S, R, K or H; especially G, S, R, K or H;
X₁₆ is G, Y, R, K or H; especially R, K or H;
X₁₇ is I;
X₁₈ is G;
X₁₉ is S, R or G; especially G;
X₂₀ is G, Y, W, V, S, R, Q, P, N, M, K, H or A; especially R, Q, P, N, K, or A; more especially R, P or K;
X₂₁ is S, Y, V, T, R, Q, P, N, M, L, K, I, H, G or F; especially R, P, K, H, G or S; more especially R, P, K, H or G; and
X₂₂ is absent.

Suitable embodiments of each of X₆, X₇, X₈, X₂₇, X₂₈, X₂₉, X₂₃, X₂₆, X₁₀ and X₁₂ are as discussed supra for Formula VII.

In some embodiments, the proteinaceous molecule is a proteinaceous molecule comprising, consisting or consisting essentially of an amino acid sequence represented by Formula IX:

Z₁CPRIGRX₆CX₇X₈X₂₇X₂₈X₂₉CX₂₃GACX₂₆CRX₁₀TX₁₂FCZ₂ (IX)

wherein:
X₆, X₇, X₈, X₂₇, X₂₈, X₂₉, X₂₃, X₂₆, X₁₀ and X₁₂ are as defined for Formula VII; and
Z₁ and Z₂ are independently absent or are independently selected from at least one of a proteinaceous moiety consisting of from about 1 to about 50 amino acid residues (and all integer residues in between), and a protecting moiety.

In some embodiments, Z₁ is absent or is a proteinaceous moiety consisting of from about 1 to about 10 amino acid residues (and all integer residues in between); especially about 2 to about 4 amino acid residues (and all integer residues in between). The amino acid residues are selected from any amino acid residues.

In some embodiments, Z₂ is absent or is a proteinaceous moiety consisting of from about 1 to about 10 amino acid residues (and all integer residues in between); especially about 1 to about 5 amino acid residues (and all integer residues in between). The amino acid residues are selected from any amino acid residues.

Suitable embodiments of X₆, X₇, X₈, X₂₇, X₂₈, X₂₉, X₂₃, X₂₆, X₁₀ and X₁₂ are as discussed supra for Formula VII.

The proteinaceous molecule of the invention, including the proteinaceous molecule of Formula I, II, III, IV, V, VI, VII, VIII or IX, and variant molecules discussed herein, is other than a peptide disclosed in Swedberg et al. (2016) J Med Chem, 59: 7287-7292; Mylne et al. (2012) The Plant Cell, 24: 2765-2778; WO 01/27147; de Veer et al. (2019) Chem Rev, 119: 12375-12421; Mahatmanto et al. (2014) Mol Biol Evol, 32(2): 392-405; Kowalska et al. (2006) Biochimica et Biophysica Acta, 1760(7): 1054-1063; Hojima et al. (1980) Thromb Res, 20(2): 163-171; Wynn et al. (1990) Biochem Biophys Res Commun, 166(3): 1406-1410; and Grzesiak et al. (2000) Biochim Biophys Acta, 1478(2): 318-324, the entire contents of which are incorporated by reference. For example, the proteinaceous molecule of the invention, including the proteinaceous molecule comprising an amino acid sequence represented by Formula I, II, III, IV, V or VI, VII, VIII or IX and variant molecules discussed herein, is other than a proteinaceous molecule comprising or consisting of the amino acid sequence of any one of SEQ ID NOs: 1 to 7, 37 to 44, 51 to 53 and 55 to 70 or a cyclized proteinaceous molecule thereof:

[SEQ ID NO: 55]
GGQCFRILKKCRRDSDCPGACICRGNGYCGSGSD;
[SEQ ID NO: 56]
GGQCSRILKKCRRDSDCPGACICRGNGYCGSGSD;
[SEQ ID NO: 57]
GGQCFRIWKKCRRDSDCPGACICRGNGYCGSGSD;
[SEQ ID NO: 58]
KGQCFRIWKKCRRDSDCPGACICRGNGYCGSGSD;
[SEQ ID NO: 59]
DGGVCPKILQRCRRDSDCPGACICRGNGYCGSGS;
[SEQ ID NO: 60]
CPRILKKCRRDSDCPGECICQGNGYC;
[SEQ ID NO: 61]
QRACPRILKKCRRDSDCPGECICQGNGYCG;
[SEQ ID NO: 62]
CPRILMPCKVDSDCLPNCTCRPNGFC;
[SEQ ID NO: 63]
CPRILMPCKVNDDCLRGCKCLSNGYC;
[SEQ ID NO: 64]
CPRILMPCKTDDDCMLDCRCLSNGYC;
[SEQ ID NO: 65]
CPRILMKCKTDRDCLTGCTCKRNGYC;
[SEQ ID NO: 66]
CPRILMKCKTDRDCLAGCTCKRNGYC;
[SEQ ID NO: 67]
CPRILMPCKSDHDCLSGCTCKRNGYC;
[SEQ ID NO: 68]
CPRILKKCRRDSDCPGECICKGNGYC;
[SEQ ID NO: 69]
CPRILKKCRRDSDCPGECICKGNGYC;
or
[SEQ ID NO: 70]
CPRILKKCRRDSDCPGECICQGNGYC.

The proteinaceous molecule of the invention, including the proteinaceous molecule of Formula I, II, III, IV, V, VI, VII, VIII or IX, and variant molecules discussed herein, is other than a proteinaceous molecule comprising or consisting of the amino acid sequence of any one of SEQ ID NOs: 1 to 7 and 51 to 53. In some embodiments, the proteinaceous molecule of the invention, including the proteinaceous molecule of Formula I, II, III, IV, V, VI, VII, VIII or IX, and variant molecules discussed herein, does not comprise an amino acid sequence of SEQ ID NO: 37 to 42. In some embodiments, the proteinaceous molecule of the invention, including the proteinaceous molecule of Formula I, II, III, IV, V, VI, VII, VIII or IX, and variant molecules discussed herein, is other than a proteinaceous molecule comprising or consisting of the amino acid sequence of any one of SEQ ID NOs: 43 or 44 or a cyclized proteinaceous molecule thereof. In some embodiments, the proteinaceous molecule of the invention, including the proteinaceous molecule of Formula I, II, III, IV, V, VI, VII, VIII or IX, and variant molecules discussed herein, is other than a proteinaceous molecule comprising or consisting of the amino acid sequence of any one of SEQ ID NOs: 55 to 70 or a cyclized proteinaceous molecule thereof.

The proteinaceous molecules of the invention have at least six cysteine residues. Preferably the proteinaceous molecules of the invention have six cysteine residues. The six cysteine residues may be bonded in pairs to form three disulfide bonds.

Cyclotides, such as MCoTI-II, are known to typically comprise six cysteine residues, with a disulfide bond connectivity between cysteine residues I and IV, II and V, and III and VI (numbered from the N-terminus). Preferably, this disulfide connectivity is present in the proteinaceous molecules of the invention, especially the proteinaceous molecules of Formula I, II, III, IV, V, VI, VII, VIII, IX and any one of SEQ ID NOs: 8 to 36, 45 to 50 and 54. When X₁ is C (such as in SEQ ID NO: 17 or 18), this cysteine does not participate in disulfide bond formation.

In some embodiments, the proteinaceous molecules comprise three disulfide bonds formed between the side chains of Cys 1 and Cys 18, Cys 8 and Cys 20, and Cys 14 and Cys 26 (numbered in accordance with Formula I starting at the N-terminal Cys residue).

Without wishing to be bound by theory, this disulfide bond connectivity forms a cystine knot motif in which a ring formed by two of the disulfide bonds and the intervening sections of the peptide backbone is pierced by the third disulfide bond. Peptides comprising a cystine knot motif have high levels of chemical and thermal stability, which may be advantageous for therapeutic use.

In some embodiments, one or more of the disulfide bonds of the proteinaceous molecule of the invention are replaced with a suitable alternative, such as a diselenide bond, a lanthionine bond, a lactam bond or a dimethylene bond. In particular embodiments, at least two cysteine residues are substituted with selenocysteine residues. The selenocysteine residues in the sequences must be positioned such that when the peptide is oxidised, a diselenide bond is produced between the side chains of two selenocysteine residues.

In some embodiments, the proteinaceous molecule is a selective antagonist of FXIIa, for example, α- and/or β-FXIIa, especially β-FXIIa (e.g. human β-FXIIa), over at least one other serine protease, such as trypsin, factor Xa (FXa), factor XIa (FXIa), thrombin, plasma kallikrein, kallikrein-related peptidase 4, plasmin, urokinase, tissue plasminogen activator and/or matriptase. In some embodiments, the proteinaceous molecule exhibits FXIIa selectivity of greater than about 2-fold, 5-fold, 10-fold, 20-fold, 50-fold or greater than about 100-fold with respect to antagonism of another serine protease. In other embodiments, the proteinaceous molecule displays at least 50-fold greater antagonism of FXIIa than another serine protease. In further embodiments, the proteinaceous molecule displays at least 100-fold greater antagonism of FXIIa than another serine protease. In still further embodiments, the proteinaceous molecule displays at least 500-fold greater antagonism of FXIIa than another serine protease. In yet further embodiments, the proteinaceous molecule displays at least 1000-fold greater antagonism of FXIIa than another serine protease.

In some embodiments, the proteinaceous molecule of the invention displays greater antagonism (e.g. affinity and/or inhibitory activity) of FXIIa than MCoTI-II, such as greater than about 2-fold, 5-fold, 10-fold, 20-fold, 50-fold or greater than about 100-fold antagonism than MCoTI-II. In particular embodiments, the proteinaceous molecule of the invention displays greater selectivity for FXIIa over at least one serine protease, such as trypsin, than MCoTI-II, such as greater than about 2-fold, 5-fold, 10-fold, 20-fold, 50-fold, 100-fold, 500-fold or greater than about 1000-fold greater selectivity for FXIIa than MCoTI-II.

In some embodiments, the proteinaceous molecule of the invention is a cyclic molecule. In particular embodiments, the proteinaceous molecule is cyclized through N-to-C cyclization (head to tail cyclization), preferably through an amide bond (i.e. an amide bond between the N- and C-termini of the linear peptide). Such peptides do not possess N- or C-terminal amino acid residues. In particular embodiments, the proteinaceous molecules of the invention have an amide-cyclized peptide backbone. In other embodiments, the proteinaceous molecules of the invention are cyclized using side-chain to side-chain cyclization, such as through a disulfide bond or a lactam bridge.

In some embodiments, the N- and C-termini are linked using a linking moiety. The linking moiety may be a peptide linker such that cyclization produces an amide-cyclized peptide backbone. Variation within the peptide sequence of the linking moiety is possible, such that the linking moiety may be modified to alter the physicochemical properties of the proteinaceous molecules and potentially reduce side effects of the proteinaceous molecules of the invention or otherwise improve the therapeutic use of the proteinaceous molecules, for example, by improving stability. The linking moiety will be of suitable length to span the distance between the N- and C-termini of the peptide without substantially altering the structural conformation of the proteinaceous molecule, for example, a peptidic linking moiety may be 1, 2, 3, 4, 5, 6, 7, 8, 9 or 10 amino acid residues in length. In some embodiments, longer or shorter peptidic linking moieties may be required. In alternative embodiments, the proteinaceous molecule is an acyclic molecule.

In some embodiments where the proteinaceous molecules of the invention comprise an N- and/or C-terminus, the proteinaceous molecules of the invention have a primary, secondary or tertiary amide, a hydrazide, a hydroxamide or a free-carboxyl group at the C-terminus and/or a primary amine or acetamide at the N-terminus. In some embodiments, the proteinaceous molecules of the invention are cyclic peptides and, thus, may not comprise N- and/or C-terminal amino acid residues. In preferred embodiments, the proteinaceous molecules of the invention have a primary amide or a free carboxyl group (C-terminal acid) at the C-terminus and a primary amine at the N-terminus, especially a free carboxyl group at the C-terminus and a primary amine at the N-terminus.

In some embodiments, the proteinaceous molecule of Formula I, II, III, IV, V, VI, VII, VIII or IX as discussed supra has at least about 80%, 81%, 82%, 83%, 84%, 85%, 86%, 87%, 88%, 89%, 90%, 91%, 92%, 93%, 94%, 95%, 96%, 97%, 98%, or 99% sequence similarity to the amino acid sequence of any one of SEQ ID NOs: 8 to 36 and 45 to 50, especially any one of SEQ ID NOs: 8 to 36 or 8 to 23, more especially any one of SEQ ID NOs: 8 to 10, 19 and 20, most especially SEQ ID NO: 8 or 19. In some embodiments, the proteinaceous molecule of Formula I, II, III IV, V, VI, VII, VIII or IX as discussed supra has at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any one of SEQ ID NOs: 8 to 36 and 45 to 50, especially any one of SEQ ID NOs: 8 to 36 or 8 to 23, more especially any one of SEQ ID NOs: 8 to 10, 19 and 20, most especially SEQ ID NO: 8 or 19. In such molecules, the variance occurs at one or more of X₁ to X₂₉, Z₁ and Z₂ when present in the subject Formula.

The present invention also contemplates proteinaceous molecules that are variants of any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19. Such “variant” proteinaceous molecules include proteinaceous molecules derived from any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, by deletion (such as from 1-10 amino acid residues and all integer amino acids therebetween) or addition of one or more amino acids (such as from 1-50 amino acid residues and all integer amino acids therebetween) to the N-terminal and/or C-terminal end of the proteinaceous molecule, deletion or addition of one or more amino acids (such as from 1-5 amino acid residues and all integer amino acids therebetween) at one or more sites in the proteinaceous molecule, or substitution of one or more amino acids at one or more sites in the proteinaceous molecule (such as from 1-10 amino acid residues and all integer amino acids therebetween). For example, in some embodiments, the variant proteinaceous molecule comprises an addition of one amino acid residue or deletion of one amino acid residue. In some embodiments, the addition or deletion occurs in the amino acid sequence between the fifth and sixth cysteine residues (i.e. Cys V and Cys VI) of the proteinaceous molecule (e.g. when numbered from the N-terminus of Formula I).

Variant proteinaceous molecules encompassed by the present invention are biologically active, that is, they continue to possess the desired biological activity of the parent proteinaceous molecule, for example, FXIIa antagonism and, in some embodiments, selectivity for FXIIa over other serine proteases as discussed herein. Such variants may result from, for example, genetic polymorphism or from human manipulation.

The proteinaceous molecules of any one of SEQ ID NOs: 8 to 36, 45 to 50 and 149 to 156; especially any one of SEQ ID NOs: 8 to 36 and 45 to 50 may be altered in various ways, including amino acid substitutions, deletions, truncations and insertions. Methods for such manipulations are generally known in the art. For example, amino acid sequence variants of any one of SEQ ID NOs: 8 to 36, 45 to 50 and 149 to 156; especially any one of SEQ ID NOs: 8 to 36 and 45 to 50 may be prepared by mutagenesis of nucleic acids encoding the amino acid sequence of any one of SEQ ID NOs: 8 to 36 and 45 to 50. Methods for mutagenesis and nucleotide sequence alterations are well known in the art. Refer to, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the proteinaceous molecule may be found in the model of Dayhoff et al., (1978) Atlas of Protein Sequence and Structure (Natl. Biomed. Res. Found., Washington, D.C.). Recursive ensemble mutagenesis (REM), a technique which enhances the frequency of functional mutants in the libraries, can be used in combination with screening assays to identify active variants (Arkin and Yourvan (1992) Proc. Natl. Acad. Sci. USA 89: 7811-7815; Delgrave et al., (1993) Protein Engineering, 6: 327-331).

Conservative substitutions, such as exchanging one amino acid with another having similar properties, may be particularly desirable. Variant proteinaceous molecules of the invention may contain conservative amino acid substitutions (e.g. 1-10 substitutions and all integers therebetween, such as 1, 2 or 3 substitutions) at various locations along their sequence, as compared to a parent or reference amino acid sequence, such as any one of SEQ ID NOs: 8 to 36, 45 to 50 and 149 to 156; especially any one of SEQ ID NOs: 8 to 36 and 45 to 50.

Variant proteinaceous molecules of the invention may contain conservative amino acid substitutions at various locations along their sequence, as compared to a parent (e.g. naturally-occurring or reference) amino acid sequence, such as any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19. A “conservative amino acid substitution” is one in which the amino acid residue is replaced with an amino acid residue having a similar side chain. Families of amino acid residues having similar side chains have been defined in the art as discussed in detail below.

Acidic: The residue has a negative charge due to loss of a proton at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with protons at physiological pH or within one or two pH units thereof (e.g. histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residue is charged at physiological pH and, therefore, includes amino acids having acidic or basic side chains, such as glutamic acid, aspartic acid, arginine, lysine and histidine.

Hydrophobic: The residue is not charged at physiological pH and the residue is repelled by aqueous solution so as to seek the inner positions in the conformation of a peptide in which it is contained when the peptide is in aqueous medium at physiological pH. Amino acids having a hydrophobic side chain include tyrosine, valine, isoleucine, leucine, methionine, norleucine, phenylalanine and tryptophan. In particular embodiments, hydrophobic amino acids include valine, leucine, isoleucine and norleucine.

Neutral/polar: The residues are not charged at physiological pH but the residue is not sufficiently repelled by aqueous solutions so that it would seek inner positions in the conformation of a peptide in which it is contained when the peptide is in aqueous medium at physiological pH. Amino acids having a neutral/polar side chain include asparagine, glutamine, cysteine, histidine, serine and threonine.

Amide-containing: The residues contain an amide in their side chain, such as glutamine and asparagine.

Aromatic: The residues contain an aromatic group in their side chain and include phenylalanine, tyrosine and tryptophan.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g. PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington DC; and by Gonnet et al., (1992), Science, 256(5062): 1443-1445), however, include proline in the same group as glycine, serine, alanine and threonine. For the purposes of the present invention, proline is not classified as a “small” amino acid unless otherwise specified. Small amino acid residues include glycine, serine, alanine and threonine.

The degree of attraction or repulsion required for classification as polar or non-polar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small amino acid residues are, of course, always non-aromatic. Dependent on their structural properties, amino acid residues may fall in two or more classes. For the naturally-occurring protein amino acids, sub-classification according to this scheme is presented in Table 1 in Section 1 supra.

Conservative amino acid substitution also includes groupings based on side chains. For example, a group of amino acids having aliphatic side chains is glycine, alanine, valine, leucine and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of an aspartic acid with a glutamic acid, a threonine with a serine, a lysine with an arginine, a tyrosine with a phenylalanine, an asparagine with a glutamine, or a similar replacement of an amino acid with a structurally related amino acid will not have a major effect on the properties of the resulting variant peptide of the invention. Whether an amino acid change results in a proteinaceous molecule that inhibits FXIIa can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
EXEMPLARY AND PREFERRED AMINO ACID SUBSTITUTIONS

| Original Residue | Exemplary Substitutions | Preferred Substitutions |
|---|---|---|
| Ala | Ser, Thr, Gly | Ser |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp | Asp |
| Gly | Ser, Thr, Ala | Ala |
| His | Asn, Gln, Lys, Arg | Arg |
| Ile | Leu, Val, Met, Ala, Phe, Nle | Leu |
| Leu | Nle, Ile, Val, Met, Ala, Phe | Ile |
| Lys | Arg, Gln, Asn | Arg |
| Met | Leu, Ile, Phe, Nle | Nle |
| Phe | Tyr, Trp, Leu, Val, Ile, Ala | Tyr |
| Pro | Gly | Gly |
| Ser | Thr, Gly, Ala | Thr |
| Thr | Ser, Gly, Ala | Ser |
| Trp | Tyr, Phe | Tyr |
| Tyr | Trp, Phe, Thr, Ser | Phe |
| Val | Ile, Leu, Met, Phe, Ala, Nle | Leu |

Alternatively, similar amino acids for making conservative substitutions can be grouped into three categories based on the identity of the side chains. The first group includes glutamic acid, aspartic acid, arginine, lysine and histidine, which all have charged side chains; the second group includes glycine, serine, threonine, cysteine, tyrosine, glutamine and asparagine; and the third group includes leucine, isoleucine, valine, alanine, proline, phenylalanine, tryptophan, methionine and norleucine, as described in Zubay, Biochemistry, third edition, Wm.C. Brown Publishers (1993).

Thus, a predicted non-essential amino acid residue in a proteinaceous molecule of the invention is typically replaced with another amino acid residue from the same side chain family. Alternatively, mutations can be introduced randomly along all or part of the coding sequence of a proteinaceous molecule of the invention, such as by saturation mutagenesis, and the resultant mutants can be screened for an activity of the parent polypeptide, as described for example herein, to identify mutants which retain that activity. Following mutagenesis of the coding sequences, the encoded proteinaceous molecule can be expressed recombinantly and its activity determined. A “non-essential” amino acid residue is a residue that can be altered from the wild-type sequence of an embodiment proteinaceous molecule of the invention without abolishing or substantially altering one or more of its activities. Suitably, the alteration does not substantially alter one of these activities, for example, the activity is at least 20%, 40%, 60%, 70% or 80% of that of the wild-type. By contrast, an “essential” amino acid residue is a residue that, when altered from the wild-type sequence of an embodiment proteinaceous molecule of the invention, results in abolition of an activity of the parent molecule such that less than 20% of the wild-type activity is present.

Accordingly, the present invention also contemplates variants of the proteinaceous molecules of any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, wherein the variants are distinguished from the parent sequence by the addition, deletion, or substitution of one or more amino acid residues. In general, variants will display at least about 80%, 81%, 82%, 83%, 84%, 85%, 86%, 87%, 88%, 89%, 90%, 91%, 92%, 93%, 94%, 95%, 96%, 97%, 98%, 99% sequence similarity to a parent or reference proteinaceous molecule sequence as, for example, set forth in any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, as determined by sequence alignment programs described elsewhere herein using default parameters. Desirably, variants will have at least 80%, 81%, 82%, 83%, 84%, 85%, 86%, 87%, 88%, 89%, 90%, 91%, 92%, 93%, 94%, 95%, 96%, 97%, 98%, 99% sequence identity to a parent or reference proteinaceous molecule sequence as, for example, set forth in any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, as determined by sequence alignment programs described herein using default parameters. Variants of any one of SEQ ID NOs: 8 to 36, 45 to 50 and 149 to 156; especially any one of SEQ ID NOs: 8 to 36 and 45 to 50; especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, which fall within the scope of a variant proteinaceous molecule of the invention, may differ from the parent molecule generally by at least 1, but by less than 10, 9, 8, 7, 6, 5, 4, 3, 2 or 1 amino acid residue(s). In some embodiments, a variant proteinaceous molecule of the invention differs from the corresponding sequence in any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, by at least 1, but by less than 10, 9, 8, 7, 6, 5, 4, 3, 2 or 1 amino acid residue(s). In some embodiments, the amino acid sequence of the variant proteinaceous molecule of the invention comprises the proteinaceous molecule of Formula I, II, III, IV, V, VI, VII, VIII or IX. In particular embodiments, the variant proteinaceous molecule of the invention inhibits an activity of FXIIa.

In such embodiments, the variant proteinaceous molecule is other than a proteinaceous molecule comprising or consisting of the amino acid sequence of any one of SEQ ID NOs: 1 to 7, 37 to 44, 51 to 53, 55 to 70, or a cyclized proteinaceous molecule thereof.

If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution.

In some embodiments, calculations of sequence similarity or sequence identity between sequences are performed as follows:

To determine the percent identity of two amino acid sequences or of two nucleic acid sequences, the sequences are aligned for optimal comparison purposes (e.g. gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 40%, more usually at least 50% or 60%, and even more usually at least 70%, 80%, 90% or 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e. conservative substitution) at the corresponding position in the second sequence, then the molecules are similar at that position.

The percent identity between the two sequences is a function of the number of identical amino acid residues shared by the sequences at individual positions, taking into account the number of gaps and the length of each gap, which need to be introduced for optimal alignment of the two sequences. By contrast, the percent similarity between the two sequences is a function of the number of identical and similar amino acid residues shared by the sequences at individual positions, taking into account the number of gaps and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wunsch, (1970, J. Mol. Biol., 48: 444-453) algorithm which has been incorporated into the GAP program in the GCG software package (Devereaux, et al. (1984) Nucleic Acids Research, 12: 387-395), using either a Blosum 62 matrix or a PAM250 matrix, and a gap weight of 16, 14, 12, 10, 8, 6, or 4 and a length weight of 1, 2, 3, 4, 5, or 6. In some embodiments, the percent identity or similarity between amino acid sequences can be determined using the algorithm of Meyers and Miller (1989, Cabios, 4: 11-17) which has been incorporated into the ALIGN program (version 2.0), using a PAM120 weight residue table, a gap length penalty of 12 and a gap penalty of 4.

The present invention also contemplates an isolated or purified proteinaceous molecule that is encoded by a polynucleotide sequence that hybridizes under stringency conditions as defined herein, especially under medium, high or very high stringency conditions, preferably under high or very high stringency conditions, to a polynucleotide sequence encoding the proteinaceous molecule of any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, or the non-coding strand thereof. The invention also contemplates an isolated nucleic acid molecule comprising a polynucleotide sequence that hybridizes under stringency conditions as defined herein, especially under medium, high or very high stringency conditions, preferably under high or very high stringency conditions, to a polynucleotide sequence encoding the proteinaceous molecule of any one of SEQ ID NOs: 8 to 36, 45 to 50 and 149 to 156; especially any one of SEQ ID NOs: 8 to 36 and 45 to 50; especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, or the non-coding strand thereof.

As used herein, the term “hybridizes under stringency conditions” describes conditions for hybridization and washing and may encompass low stringency, medium stringency, high stringency and very high stringency conditions.

Guidance for performing hybridization reactions can be found in Ausubel, et al. (1998) Current Protocols in Molecular Biology (John Wiley and Sons, Inc.), in particular sections 6.3.1-6.3.6. Both aqueous and non-aqueous methods can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% sodium dodecyl sulfate (SDS) for hybridization at 65° C., and (i) 2× sodium chloride/sodium citrate (SSC), 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6×SSC at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In some aspects of the present invention, there is provided a proteinaceous molecule of the invention that is encoded by a polynucleotide sequence that hybridizes under high stringency conditions to a polynucleotide sequence encoding the proteinaceous molecule of any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, or the non-coding strand thereof. In certain embodiments, the isolated or purified proteinaceous molecule of the invention is encoded by a polynucleotide sequence that hybridizes under very high stringency conditions to a polynucleotide sequence encoding the proteinaceous molecule of any one of SEQ ID NOs: 8 to 36, 45 to 50 and 149 to 156; especially any one of SEQ ID NOs: 8 to 36 and 45 to 50; more especially any one of SEQ ID NOs: 8 to 36; more especially any one of SEQ ID NOs: 8 to 23; more especially any one of SEQ ID NOs: 8 to 10, 19 and 20; most especially SEQ ID NO: 8 or 19, or the non-coding strand thereof. One embodiment of very high stringency conditions includes hybridizing 0.5 M sodium phosphate, 7% SDS at 65° C., followed by one or more washes at 0.2×SSC, 1% SDS at 65° C. In some embodiments, the amino acid sequence of the variant proteinaceous molecule of the invention comprises the amino acid sequence of Formula I, II, III, IV, V, VI, VII, VIII or IX. In particular embodiments, the variant proteinaceous molecule of the invention inhibits an activity of FXIIa.

Other stringency conditions are well known in the art and a person skilled in the art will recognize that various factors can be manipulated to optimize the specificity of the hybridization. Optimization of the stringency of the final washes can serve to ensure a high degree of hybridization. For detailed examples, see Ausubel, et al. (1998) Current Protocols in Molecular Biology (John Wiley and Sons, Inc.), in particular pages 2.10.1 to 2.10.16 and Sambrook, et al. (1989) Molecular Cloning: A Laboratory Manual (Cold Spring Harbour Press), in particular Sections 1.101 to 1.104.

While stringent washes are typically carried out at temperatures from about 42° C. to 68° C., a person skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel, et al. (1998) Current Protocols in Molecular Biology (John Wiley and Sons, Inc.) at page 2.10.8). In general, the Tₘ of a perfectly matched duplex of DNA may be predicted as an approximation by the formula:

Tₘ=81.5+16.6(log₁₀ M)+0.41(% G+C)−0.63(% formamide)−(600/length)

wherein: M is the concentration of Na+, preferably in the range of 0.01 M to 0.4 M; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g. a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrrolidone and 0.1% BSA), 0.1% SDS and 200 mg/mL denatured salmon sperm DNA) containing labeled probe. The membrane is then subjected to two sequential medium stringency washes (i.e. 2×SSC, 0.1% SDS for 15 min at 45° C., followed by 2×SSC, 0.1% SDS for 15 min at 50° C.), followed by two sequential higher stringency washes (i.e. 0.2×SSC, 0.1% SDS for 12 min at 55° C. followed by 0.2×SSC and 0.1% SDS solution for 12 min at 65-68° C.

The proteinaceous molecules of the invention may also encompass modified amino acid residues. Modified amino acid residues may include residues with modified side chains, N-methyl amino acids, α-methyl amino acids, residues with acetylated N-termini, beta amino acids, and the like.

Examples of side chain modifications include modifications of amino groups, such as by acetylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with sodium borohydride; reductive alkylation by reaction with an aldehyde followed by reduction with sodium borohydride; and trinitrobenzylation of amino groups with 2,4,6-trinitrobenzene sulfonic acid (TNBS). The carboxyl group may be modified by carbodiimide activation through O-acylisourea formation followed by subsequent derivatization, for example, to a corresponding amide. The guanidine group of arginine residues may be modified by formation of heterocyclic condensation products with reagents such as 2,3-butanedione, phenylglyoxal and glyoxal. Tryptophan residues may be modified, for example, by alkylation of the indole ring with 2-hydroxy-5-nitrobenzyl bromide or sulfonyl halides, or by oxidation with N-bromosuccinimide. Tyrosine residues may be modified by nitration with tetranitromethane to form a 3-nitrotyrosine derivative.

Suitable modified arginine residues include, but are not limited to, N_{ω}-carboxymethyl-L-arginine, N_{ω}-carboxyethyl-L-arginine, N_{α}-acetyl-L-arginine, di(phenylglyoxal)-L-arginine, N-methylarginine, α-methylarginine, β-arginine, N′-nitro-L-arginine, N′,N″-dimethyl-L-arginine, N′,N″-diethyl-L-arginine and L-homoarginine.

Suitable modified lysine residues include, but are not limited to, Nε-carboxycarbonyl-L-lysine, Nε-succinimidyl-L-lysine, 2-amino-6-(2-hydroxyacetamido)hexanoic acid, Nε-3-hydroxypropyl-L-lysine, ornithine, Nε-allyloxycarbonyl-L-lysine, N-methyllysine, α-methyllysine, β-lysine, N_{α}-acetyl-L-lysine, Nε-acetyl-L-lysine, Nε-methyl-L-lysine, Nε-dimethyl-L-lysine and Nε-formyl-L-lysine.

Suitable modified alanine residues include, but are not limited to, N-methylalanine, α-methylalanine (2-aminoisobutyric acid), β-alanine, N_{α}-acetyl-L-alanine, α-aminobutyric acid (or 2-aminobutyric acid, Abu), homoalanine and β-homoalanine.

Suitable modified leucine residues include, but are not limited to, α-methylleucine, N-methylleucine, β-leucine, t-butylglycine, homoleucine, N_{α}-acetyl-L-leucine and β-homoleucine.

Suitable modified glutamine residues include, but are not limited to, α-methylglutamine, N_{α}-methylglutamine, N_{γ}-methylglutamine, β-glutamine, homoglutamine, N_{α}-acetyl-L-glutamine and β-homoglutamine.

Exemplary modified asparagine residues include N_{β}-methyl-N_{β}-methoxy-asparagine, α-methylasparagine, N_{α}-methylasparagine, N_{β}-methylasparagine, β-asparagine, homoasparagine, N_{α}-acetyl-L-asparagine and β-homoasparagine.

Modified glycine residues include, but are not limited to, N-methylglycine, β-homoglycine and N_{α}-acetyl-L-glycine.

Modified serine residues may include N-methylserine, α-methylserine, β-serine, N_{α}-acetyl-L-serine, isoserine, O-methylserine, homoserine and β-homoserine.

Exemplary modified threonine residues include N-methylthreonine, α-methylthreonine, β-threonine, N_{α}-acetyl-L-threonine, O-methylthreonine, homothreonine and β-homothreonine.

Suitable modified methionine residues include, but are not limited to, norleucine, N-methylmethionine, α-methylmethionine, β-methionine, N_{α}-acetyl-L-methionine, methionine sulfoxide, methionine sulfone, selenomethionine, homomethionine and β-homomethionine.

Exemplary modified proline residues include α-methylproline, β-proline, N_{α}-acetyl-L-proline, 4-phenoxy-pyrrolidine-2-carboxylic acid, 5,5-dimethylpyrrolidine-2-carboxylic acid, 5-methylpyrrolidine-2-carboxylic acid, homoproline and β-homoproline.

Suitable modified isoleucine residues include, but are not limited to, α-methylisoleucine, N-methylisoleucine, β-isoleucine, homoisoleucine, N_{α}-acetyl-L-isoleucine, β-methylisoleucine and β-homoisoleucine.

Modified valine residues may include, but are not limited to, norvaline, α-methylvaline, N-methylvaline, β-valine, β-homovaline and N_{α}-acetyl-L-valine.

Suitable modified phenylalanine residues include, but are not limited to, α-methylphenylalanine, N-methylphenylalanine, β-phenylalanine, β-methylphenylalanine, β,β-dimethylphenylalanine, β-hydroxyphenylalanine, homophenylalanine, N_{α}-acetyl-L-phenylalanine, β-homophenylalanine, 4-fluoro-L-phenylalanine (4-F-Phe) and 4-methyl-L-phenylalanine (4-Me-Phe).

Exemplary modified tyrosine residues include α-methyltyrosine, N-methyltyrosine, β-tyrosine, β-methyltyrosine, β,β-dimethyltyrosine, β-hydroxytyrosine, homotyrosine, O-methylhomotyrosine, N_{α}-acetyl-L-tyrosine, O-methyltyrosine, O-ethyltyrosine, m-tyrosine and β-homotyrosine.

Suitable modified tryptophan residues include, but are not limited to, α-methyltryptophan, N-methyltryptophan, β-tryptophan, β-methyltryptophan, homotryptophan, N-formyl-tryptophan, 2-methyltryptophan, N_{α}-acetyl-L-tryptophan and β-homotryptophan.

Suitable modified glutamic acid residues include, but are not limited to, N-methylglutamic acid, α-methylglutamic acid, β-glutamic acid, N_{α}-acetyl-L-glutamic acid, glutamic acid γ-methyl ester, γ-carboxy glutamic acid, homoglutamic acid and β-homoglutamic acid.

Suitable modified aspartic acid residues include, but are not limited to, N-methylaspartic acid, α-methylaspartic acid, β-aspartic acid, N_{α}-acetyl-L-aspartic acid, aspartic acid β-methyl ester and β-homoaspartic acid.

Suitable modified cysteine residues include, but are not limited to, N-methylcysteine, α-methylcysteine, N-acetylcysteine, β-cysteine, β-methylcysteine and homocysteine.

The proteinaceous molecules of the invention also encompass a proteinaceous molecule comprising unnatural amino acid residues and/or their derivatives during peptide synthesis and the use of cross-linkers and other methods which impose conformational constraints on the proteinaceous molecules.

Examples of incorporating unnatural amino acids and derivatives during peptide synthesis include, but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, 2-aminobutyric acid, ornithine, N-acetyl-L-ornithine, sarcosine, 2-thienyl alanine, 4-F-Phe, 4-Me-Phe and/or D-isomers of amino acids. A list of unnatural amino acids contemplated by the present invention is shown in Table 3, in addition to the modified resides discussed supra.

TABLE 3
EXEMPLARY UNNATURAL AMINO ACIDS
NON-CONVENTIONAL AMINO ACIDS

| | |
|---|---|
| α-aminobutyric acid/2- | L-N-methylalanine |
| aminobutyric acid | |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamine | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-asparagine | α-methylcyclohexylalanine |
| D-α-methylalanine | α-methylcylcopentylalanine |
| D-α-methylarginine | α-methyl-α-naphthylalanine |
| D-α-methylasparagine | α-methylpenicillamine |
| D-α-methylaspartate | N-(4-aminobutyl)glycine |
| D-α-methylcysteine | N-(2-aminoethyl)glycine |
| D-α-methylglutamine | N-(3-aminopropyl)glycine |
| D-α-methylhistidine | N-amino-α-methylbutyrate |
| D-α-methylisoleucine | α-naphthylalanine |
| D-α-methylleucine | N-benzylglycine |
| D-α-methyllysine | N-(2-carbamylethyl)glycine |
| D-α-methylmethionine | N-(carbamylmethyl)glycine |
| D-α-methylornithine | N-(2-carboxyethyl)glycine |
| D-α-methylphenylalanine | N-(carboxymethyl)glycine |
| D-α-methylproline | N-cyclobutylglycine |
| D-α-methylserine | N-cycloheptylglycine |
| D-α-methylthreonine | N-cyclohexylglycine |
| D-α-methyltryptophan | N-cyclodecylglycine |
| D-α-methyltyrosine | L-α-methyllysine |
| L-α-methylleucine | L-α-methylnorleucine |
| L-α-methylmethionine | L-α-methylornithine |
| L-α-methylnorvaline | L-α-methylproline |
| L-α-methylphenylalanine | L-α-methylthreonine |
| L-α-methylserine | L-α-methyltyrosine |
| L-α-methyltryptophan | L-N-methylhomophenylalanine |
| L-α-methylvaline | N-(N-(3,3-diphenylpropyl |
| | carbamylmethyl)glycine |
| N-(N-(2,2-diphenylethyl | L-selenocysteine |
| carbamylmethyl)glycine | |
| 1-carboxy-1-(2,2-diphenyl-ethyl | L-selenomethionine |
| amino)cyclopropane | |
| D-selenocysteine | L-O-methyl homoserine |
| L-telluromethionine | L-ornithine |
| L-S-ethyl cysteine | N_{ε}-methyl-L-lysine |
| N_{ε}-acetyl-L-lysine | N_{ε}-formyl-L-lysine |
| N_{ε}-dimethyl-L-lysine | D-norvaline |
| D-norleucine | N_{δ}-acetyl-L-ornithine |
| 4-F-Phe | 4-Me-Phe |

Additional amino acids or other substituents may be added to the N- or C-termini, if present, of the proteinaceous molecules of the invention. For example, the proteinaceous molecules of the invention may form part of a longer sequence with additional amino acids added to either or both of the N- and C-termini.

Proteinaceous molecules with high levels of stability may be desired, for example, to increase the half-life of the proteinaceous molecule in a subject. Thus, in some embodiments, the proteinaceous molecules of the invention comprise a stabilizing or protecting moiety, for example, when the proteinaceous molecule is acyclic. The stabilizing or protecting moiety may be conjugated at any point on the proteinaceous molecule. The stabilizing or protecting moiety may be any moiety which delays or prevents substantial degradation of the proteinaceous molecule. A skilled person will be well aware of suitable stabilizing or protecting moieties which may be used. Exemplary stabilizing or protecting moieties include, but are not limited to, a peptide or protein such as an albumin including human serum albumin or a fragment or variant thereof, a glycine-rich homo-amino-acid polymer, a PAS sequence comprising a combination of alanine, serine and proline residues, the C-terminal peptide (CTP) of the β subunit of human chorionic gonadotropin or fragment or variant thereof, transferrin or a fragment or variant thereof, an albumin binding moiety, which comprises an albumin binding peptide, a bacterial albumin binding domain, an albumin-binding antibody fragment, or any combinations thereof, or an XTEN polypeptide (an extended length polypeptide with a non-naturally occurring, substantially non-repetitive sequence that is composed mainly of small hydrophilic amino acids, with the sequence having a low degree or no secondary or tertiary structure under physiologic conditions); an Fc region or single chain Fc region comprising a functional neonatal Fc receptor (FcRn) binding partner comprising an Fc domain, variant, or fragment thereof; a polymer such as a polyethylene glycol (PEG), a polysialic acid or a derivative thereof, hydroxyethyl starch or a derivative thereof, ethylene glycol/propylene glycol copolymers, carboxymethylcellulose, dextran or polyvinyl alcohol; a glycan or polysaccharide; a lipid moiety for example, a C₆-C₂₀ fatty acyl group; or a capping moiety, including an acetyl group, pyroglutamate or an amino group.

In some embodiments, the protecting or stabilizing moiety is a PEG. The PEG can be of any molecular weight, and can be branched or unbranched. In one embodiment, the molecular weight is between about 1 kDa and about 100 kDa for ease in handling and manufacturing. Other sizes can be used, depending on the desired profile (e.g. the duration of sustained release desired, the effects, if any on biological activity, the ease in handling and other known effects of the polyethylene glycol to a peptide or protein). For example, the polyethylene glycol can have an average molecular weight of about 1, 5, 10, 15, 20, 25, 30, 35, 40, 45, 50, 55, 60, 65, 70, 75, 80, 85, 90, 95, 100, 200, 500, 1000, 1500, 2000, 2500, 3000, 3500, 4000, 4500 or 5000 kDa.

In some embodiments, the polyethylene glycol can have a branched structure. Branched polyethylene glycols are described, for example, in U.S. Pat. No. 5,643,575; Morpurgo et al. (1996. Appl. Biochem. Biotechnol. 56:59-72); Vorobjev et al. (1999. Nucleosides Nucleotides 18:2745-2750); and Caliceti et al. (1999. Bioconjug. Chem. 10:638-646).

In some embodiments, the protecting or stabilizing moiety is a lipid moiety. The lipid moiety may be a lipid moiety comprising 6 to 24 carbon atoms in the alkyl chain (and all integers therebetween); especially 8 to 22 carbon atoms; most especially 10 to 20 carbon atoms (e.g. a C₆-C₂₀ fatty acyl group). For example, the lipid moiety may be hexanoyl (C₆), heptanoyl (C₇), octanoyl (C₈), nonanoyk (C₉), decanoyl (C₁₀), undecanoyl (C₁₁), dodecanoyl (C₁₂), tridecanoyl (C₁₃), tetradecanoyl (C₁₄), pentadecanoyl (C₁₅), hexadecanoyl (C₁₆), heptadecanoyl (C₁₇) or octadecanoyl (C₁₈). In particular embodiments, the lipid moiety is hexanoyl (C₆), octanoyl (C₈), decanoyl (C₁₀), dodecanoyl (C₁₂), tetradecanoyl (C₁₄), hexadecanoyl (C₁₆) or octadecanoyl (C₁₈); especially tetradecanoyl, hexadecanoyl or octadecanoyl. While the lipid moiety may be directly conjugated to the proteinaceous molecule, in some embodiments, the lipid moiety is conjugated via a linker to the proteinaceous molecule, such as a PEG linker (e.g. a PEG containing from 4 to 12 ethylene glycol groups).

In preferred embodiments, the acetyl group and/or pyroglutamate are conjugated to the N-terminal amino acid residue of the proteinaceous molecule. In particular embodiments, the N-terminus of the proteinaceous molecule is a pyroglutamide or acetamide. In some embodiments, the amino group is conjugated to the C-terminal amino acid residue of the proteinaceous molecule. In particular embodiments, the proteinaceous molecule of the invention has a primary amide at the C-terminus.

When present the PEG or lipid moiety may be, for example, conjugated to the N-terminal or C-terminal amino acid residue of the proteinaceous molecule or through the amine of a lysine side-chain, especially through the N-terminal amino acid residue, such as through the α-amino group or through the amino group of a lysine side-chain (i.e. the ε-amino group).

In particular embodiments, the proteinaceous molecule of the invention has a primary amide or a free carboxyl group (acid) at the C-terminus and a primary amine or acetamide at the N-terminus; especially a C-terminal acid, and an N-terminal amine.

While the protecting or stabilizing moiety may be attached to the N- and/or C-terminus of the proteinaceous molecule, the moiety may also be attached to the proteinaceous molecule through a side-chain of an amino acid residue, such as through the amino group in the side chain of an amine- or amide-containing amino acid residue, such as lysine, arginine, glutamine and asparagine or other suitably modified side chain, especially through a lysine side chain.

The proteinaceous molecules of the invention may be isolated or purified.

The proteinaceous molecules of the invention may also be in the form of salts or prodrugs. The salts of the proteinaceous molecules of the present invention are preferably pharmaceutically acceptable, but it will be appreciated that non-pharmaceutically acceptable salts also fall within the scope of the present invention.

The proteinaceous molecules may be in crystalline form and/or in the form of solvates, for example, hydrates. Solvation may be performed using methods known in the art.

The present invention also contemplates nucleic acid molecules which encode a proteinaceous molecule of the invention. Thus, in a further aspect of the present invention, there is provided an isolated nucleic acid molecule comprising a polynucleotide sequence that encodes a proteinaceous molecule of the invention or is complementary to a polynucleotide sequence that encodes a proteinaceous molecule of the invention, such as the proteinaceous molecule comprising, consisting or consisting essentially of a sequence represented by Formula I, II, III, IV, V, VI, VII, VIII, IX or any one of SEQ ID NOs: 8 to 36, 45 to 50, 54 and 149 to 156; especially any one of SEQ ID NOs: 8 to 36, 45 to 50 and 54 as described herein.

The isolated nucleic acid molecules of the present invention may be DNA or RNA. When the nucleic acid is in DNA form, it may be genomic DNA or cDNA. RNA forms of the nucleic acid molecules of the present invention are generally mRNA.

Although the nucleic acid molecules are typically isolated, in some embodiments the nucleic acid molecules may be integrated into, ligated to, or otherwise fused or associated with other genetic molecules, such as an expression vector. Generally an expression vector includes transcriptional and translational regulatory nucleic acid operably linked to the polynucleotide sequence. Accordingly, in another aspect of the invention, there is provided an expression vector comprising a polynucleotide sequence that encodes a proteinaceous molecule of the invention, such as a proteinaceous molecule comprising, consisting or consisting essentially of a sequence represented by Formula I, II, III, IV, V, VI, VII, VIII, IX or any one of SEQ ID NOs: 8 to 36, 45 to 50, 54 and 149 to 156; especially any one of SEQ ID NOs: 8 to 36, 45 to 50 and 54 as described herein.

In some embodiments, the proteinaceous molecules of the invention may be produced inside a cell by introduction of one or more expression constructs, such as an expression vector, that comprise a polynucleotide sequence that encodes a proteinaceous molecule of the invention.

The invention contemplates recombinantly producing the proteinaceous molecules of the invention inside a host cell, such as a mammalian cell (e.g. Chinese hamster ovary (CHO) cell, mouse myeloma (NSO) cell, baby hamster kidney (BHK) cell or human embryonic kidney (HEK293) cell), yeast cell (e.g. Pichia pastoris cell, Saccharomyces cerevisiae cell, Schizosaccharomyces pombe cell, Hansenula polymorpha cell, Kluyveromyces lactis cell, Yarrowia lipolytica cell or Arxula adeninivorans cell), insect cell (e.g. Spodoptera frugiperda cell, such as an Sf9 cell) or bacterial cell (e.g. Escherichia coli cell, Corynebacterium glutamicum or Pseudomonas fluorescens cell).

As described, for example, in U.S. Pat. No. 5,976,567, the expression of natural or synthetic nucleic acids is typically achieved by operably linking a polynucleotide sequence encoding a proteinaceous molecule of the invention to a regulatory element (e.g. a promoter, which may be either constitutive or inducible), suitably incorporating the construct into an expression vector and introducing the vector into a suitable host cell. Typical vectors contain transcription and translation terminators, transcription and translation initiation sequences and promoters useful for regulation of the expression of the nucleic acid. The vectors optionally comprise generic expression cassettes containing at least one independent terminator sequence, sequences permitting replication of the cassette in eukaryotes, prokaryotes or both, (e.g. shuttle vectors) and selection markers for both prokaryotic and eukaryotic systems. Vectors may be suitable for replication and integration in prokaryotes, eukaryotes, or both. See, Giliman and Smith (1979), Gene, 8: 81-97; Roberts et al. (1987) Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), Molecular Cloning—a Laboratory Manual (2nd ed.) Vol. 1-3, Cold Spring Harbor Laboratory, Cold Spring Harbor Press, N.Y.; and Ausubel et al., (1994) Current Protocols in Molecular Biology, eds., Current Protocols, a joint venture between Greene Publishing Associates, Inc. and John Wiley & Sons, Inc. (Supplement).

Expression vectors containing regulatory elements from eukaryotic viruses such as retroviruses are typically used for expression of nucleic acid sequences in eukaryotic cells. Examplary vectors include SV40 vectors such as pSVT7 and pMT2, vectors derived from bovine papilloma virus such as pBV-1MTHA, and vectors derived from Epstein Bar virus such as pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumour virus promoter, Rous sarcoma virus promoter, polyhedrin promoter, or other promoters shown effective for expression in eukaryotic cells.

While a variety of vectors may be used, it should be noted that viral expression vectors are useful for modifying eukaryotic cells because of the high efficiency with which the viral vectors transfect target cells and integrate into the target cell genome. Illustrative expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000) Curr. Opin. Biotechnol., 11(2): 205-208; Vigna and Naldini (2000) J. Gene Med., 2(5): 308-316; Kay et al. (2001) Nat. Med., 7(1): 33-40; Athanasopoulos et al. (2000) Int. J. Mol. Med., 6(4): 363-375; and Walther and Stein (2000) Drugs, 60(2): 249-271.

The polypeptide or peptide-encoding portion of the expression vector may comprise a naturally-occurring sequence or a variant thereof, which has been engineered using recombinant techniques. In one example of a variant, the codon composition of a polynucleotide encoding a proteinaceous molecule of the invention is modified to permit enhanced expression of the proteinaceous molecule of the invention in a mammalian host using methods that take advantage of codon usage bias, or codon translational efficiency in specific mammalian cell or tissue types as set forth, for example, in International Publications WO 99/02694 and WO 00/42215. Briefly, these latter methods are based on the observation that translational efficiencies of different codons vary between different cells or tissues and that these differences can be exploited, together with codon composition of a gene, to regulate expression of a protein in a particular cell or tissue type. Thus, for the construction of codon-optimized polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector is compatible with the cell in which it is introduced such that the proteinaceous molecule of the invention is expressible by the cell. The expression vector is introduced into the cell by any suitable means which will be dependent on the particular choice of expression vector and cell employed. Such means of introduction are well-known to those skilled in the art. For example, introduction can be effected by use of contacting (e.g. in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g. Lipofectin®, Lipofectamine™, and the like, supplied by Invitrogen Waltham MA, USA).

The proteinaceous molecules may be prepared using any suitable method, such as chemical synthesis or recombinant DNA techniques. In some embodiments, the proteinaceous molecules are prepared using standard peptide synthesis methods, such as solution synthesis or solid phase synthesis. The chemical synthesis of the proteinaceous molecules may be performed manually or using an automated synthesizer. For example, the linear peptides may be synthesized using solid phase peptide synthesis using either Boc or Fmoc chemistry, as described in Merrifield (1963) J Am Chem Soc, 85(14): 2149-2154; Schnolzer, et al. (1992) Int J Pept Protein Res, 40: 180-193; Cardoso, et al. (2015) Mol Pharmacol, 88(2): 291-303; and Kumar et al. (2020) ACS Omega, 5: 2345-2354, the entire contents of which are incorporated by reference. Following deprotection and cleavage from the solid support, the linear peptides are purified using suitable methods, such as preparative chromatography, and disulfide bonds are formed using oxidation where appropriate. Suitable conditions for oxidation of the peptide will be readily determined by a person skilled in the art.

In some embodiments, the proteinaceous molecules of the invention may be cyclized. Cyclization may be performed using several techniques, for example, as described in Davies (2003) J Pept Sci, 9: 471-501; or Thongyoo et al. (2006) Chem Commun (Camb), 27: 2848-2850, the contents of which are incorporated by reference. For example, N-to-C cyclization may be conducted in the solution phase, using a dilute solution of the linear peptide in the presence of a coupling agent such as BOP (1-benzotriazole-tris-dimethyl aminophosphonium hexafluorophosphate), PyBOP (1-benzotriazolyloxy-tris-pyrrolidino phosphonium hexafluorophosphate), PyAOP (7-azabenzotriazol-1-yloxy tris pyrrolidino phosphonium hexafluorophosphate), AOP (7-azabenzotriazol-1-yloxy-tris-dimethyl aminophosphonium hexafluorophosphate), HBTU (0-(benzotriazol-1-yl)-1,1,3,3-tetramethyl uronium hexafluorophosphate), TBTU (0-(benzotriazol-1-yl)-1,1,3,3-tetramethyl uronium tetrafluoroborate), HATU (0-(7-azabenzotriazol-1-yl)-1,1,3,3-tetramethyl uronium hexafluorophosphate), HAPyU (0-(7-azabenzotriazol-1-yl)-1,1,3,3-tetramethylene uronium hexafluorophosphate), HAPipU (0-(7-azabenzotriazol-1-yl)-1,1,3,3-pentamethylene uranium hexafluorophosphate), DCC (N,N′-dicyclohexylcarbodiimide), DIC (N,N′-diisopropylcarbodiimide), and/or EDC [1-ethyl-3-(3′-dimethylaminopropyl)carbodiimide hydrochloride]. The cyclized peptide may then be deprotected (i.e. the side chain protecting groups may then be removed) using standard techniques, followed by purification using suitable methods, such as preparative chromatography. Alternatively, N-to-C cyclization may be achieved on resin using a suitable coupling agent, such as those described above, and a suitable resin, such as a Kaiser oxime resin, and/or linker (e.g. a safety catch linker), or via native chemical ligation as described in Thongyoo et al. (2006) Chem Commun (Camb), 27: 2848-2850, the entire contents of which is incorporated by reference.

In some embodiments, the proteinaceous molecules of the invention are prepared using recombinant DNA techniques. For example, the proteinaceous molecules of the invention may be prepared by a procedure including the steps of: (a) preparing a construct comprising a polynucleotide sequence that encodes the proteinaceous molecule of the invention and that is operably linked to a regulatory element; (b) introducing the construct into a host cell; (c) culturing the host cell to express the polynucleotide sequence to thereby produce the encoded proteinaceous molecule of the invention; and (d) isolating the proteinaceous molecule of the invention from the host cell. The proteinaceous molecule of the present invention may be prepared recombinantly using standard protocols, for example, as described in Klint, et al. (2013) PLOS One, 8(5): e63865; Sambrook, et al. (1989) Molecular Cloning: A Laboratory Manual (Cold Spring Harbour Press), in particular Sections 16 and 17; Ausubel, et al. (1998) Current Protocols in Molecular Biology (John Wiley and Sons, Inc.), in particular Chapters 10 and 16; and Coligan, et al. (1997) Current Protocols in Protein Science (John Wiley and Sons, Inc.), in particular Chapters 1, 5 and 6. Under some circumstances it may be desirable to undertake oxidative disulfide bond formation of the expressed peptide after peptide expression. This may be preceded by a reductive step to provide the linear peptide. Suitable conditions for reduction and oxidation of the peptide will be readily determined by a person skilled in the art.

### 4. Compositions

In accordance with the present invention, the proteinaceous molecules are also useful in compositions and methods for treating or inhibiting the development of a condition associated with FXIIa activity, including thromboembolism-associated conditions such as acute coronary syndrome, stroke, deep vein thrombosis and pulmonary embolism, a thrombosis, a thrombosis-associated hematologic disorder, such as sickle cell disease or thrombophilia, or an inflammatory condition or a condition related to the kallikrein-kinin system, such as hereditary angioedema, multiple sclerosis, rheumatoid arthritis or lupus, or for treating or inhibiting thrombus and/or embolus formation. Thus, in some embodiments, the proteinaceous molecules may be in the form of a pharmaceutical composition, wherein the pharmaceutical composition comprises, consists or consists essentially of a proteinaceous molecule of the invention and a pharmaceutically acceptable carrier or diluent.

The proteinaceous molecule may be formulated into the pharmaceutical composition as a neutral or salt form.

As will be appreciated by those skilled in the art, the choice of pharmaceutically acceptable carrier or diluent will be dependent on the route of administration and on the nature of the condition and subject to be treated. The particular carrier or delivery system and route of administration may be readily determined by a person skilled in the art. The carrier or delivery system and route of administration should be carefully selected to ensure that the activity of the proteinaceous molecule is not depleted during preparation of the formulation and the proteinaceous molecule is able to reach the site of action intact. The pharmaceutical compositions of the invention may be administered through a variety of routes including, but not limited to, oral, rectal, topical, intranasal, intraocular, transmucosal, intestinal, enteral, intramuscular, subcutaneous, intramedullary, intrathecal, intraventricular, intracerebral, intravaginal, intravesical, intravenous or intraperitoneal administration; especially oral, intravenous, intramuscular, subcutaneous, intrathecal, intraventricular, intracerebral or intraperitoneal administration.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

A person skilled in the art will readily be able to determine appropriate formulations for the proteinaceous molecules using conventional approaches. Techniques for formulation and administration may be found in, for example, Remington: The Science and Practice of Pharmacy, Loyd V. Allen, Jr (Ed), The Pharmaceutical Press, London, 22^{nd} Edition, September 2012.

Identification of preferred pH ranges and suitable excipients, such as antioxidants, is routine in the art, for example, as described in Katdare and Chaubel (2006) Excipient Development for Pharmaceutical, Biotechnology and Drug Delivery Systems (CRC Press). Buffer systems are routinely used to provide pH values of a desired range and may include, but are not limited to, carboxylic acid buffers, such as acetate, citrate, lactate, tartrate and succinate; glycine; histidine; phosphate; tris(hydroxymethyl)aminomethane (Tris); arginine; sodium hydroxide; glutamate; and carbonate buffers. Suitable antioxidants may include, but are not limited to, phenolic compounds such as butylated hydroxytoluene (BHT) and butylated hydroxyanisole; vitamin E; ascorbic acid; reducing agents such as methionine or sulfite; metal chelators such as ethylene diamine tetraacetic acid (EDTA); cysteine hydrochloride; sodium bisulfite; sodium metabisulfite; sodium sulfite; ascorbyl palmitate; lecithin; propyl gallate; and alpha-tocopherol.

For injection, the proteinaceous molecule may be formulated in an aqueous solution, suitably in physiologically compatible buffers such as Hanks' solution, Ringer's solution, dextrose solution or physiological saline buffer, such as phosphate buffered saline (PBS). For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the proteinaceous molecule can be formulated readily using pharmaceutically acceptable carriers well known in the art into dosages suitable for oral administration, which is also contemplated for the practice of the present invention. Such carriers enable the proteinaceous molecules of the invention to be formulated in dosage forms such as tablets, pills, capsules, liquids, gels, syrups, slurries, suspensions and the like, for oral ingestion by a patient to be treated. These carriers may be selected from sugars, chitosan, starches, cellulose and its derivatives, malt, gelatin, talc, calcium sulfate, vegetable oils, synthetic oils, polyols, alginic acid, phosphate buffered solutions, emulsifiers, isotonic saline and pyrogen-free water.

Pharmaceutical formulations for parenteral administration include aqueous solutions of the composition in water-soluble form. Additionally, suspensions of the proteinaceous molecule may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilizers or agents that increase the solubility of the proteinaceous molecules to allow for the preparation of highly concentrated solutions.

Sterile solutions may be prepared by combining the proteinaceous molecule in the required amount in the appropriate solvent with other excipients as described above as required, followed by sterilization, such as filtration. Generally, dispersions are prepared by incorporating the various sterilized active agents into a sterile vehicle which contains the basic dispersion medium and the required excipients as described above. Sterile dry powders may be prepared by vacuum- or freeze-drying a sterile solution comprising the active agents and other required excipients as described above.

Pharmaceutical preparations for oral use can be obtained by combining the proteinaceous molecules with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof, such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The proteinaceous molecules may be incorporated into modified-release preparations and formulations, for example, polymeric microsphere formulations, and oil- or gel-based formulations.

The proteinaceous molecules may be administered in a local rather than systemic manner, such as by injection directly into a tissue, which is preferably subcutaneous or omental tissue, often in a depot or sustained release formulation. In other embodiments, the proteinaceous molecule is systemically administered.

Furthermore, the proteinaceous molecule may be administered in a targeted drug delivery system, such as in a particle which is suitable targeted to and taken up selectively by a cell or tissue. In some embodiments, the proteinaceous molecule is contained or otherwise associated with a vehicle selected from liposomes, micelles, dendrimers, biodegradable particles, artificial DNA nanostructure, lipid-based nanoparticles and carbon or old nanoparticles. In illustrative examples of this type, the vehicle is selected from poly(lactic acid) (PLA), poly(glycolic acid) (PGA), poly(lactic-co-glycolic acid) (PLGA), poly(ethylene glycol) (PEG), PLA-PEG copolymers and combinations thereof.

In cases of local administration or selective uptake, the effective local concentration of the agent may not be related to plasma concentration.

It is advantageous to formulate the compositions in dosage unit form for ease of administration and uniformity of dosage. The determination of the novel dosage unit forms of the present invention is dictated by and directly dependent on the unique characteristics of the active material, the particular therapeutic effect to be achieved and the limitations inherent in the art of compounding active materials for the treatment of disease in living subjects having a diseased condition in which bodily health is impaired as herein disclosed in detail.

While the proteinaceous molecule of the invention may be the sole active ingredient administered to the subject, the administration of other active ingredients concurrently with said proteinaceous molecule is within the scope of the invention. For example, in some embodiments, the proteinaceous molecule may be administered concurrently with one or more anti-inflammatory agents, or anticoagulants. The proteinaceous molecule may be therapeutically used after the other active ingredient or may be therapeutically used together with the other active ingredient. The proteinaceous molecule may be administered separately, simultaneously or sequentially with the other active ingredient.

Accordingly, in another aspect of the invention, there is provided a composition comprising a proteinaceous molecule of the invention and an anti-inflammatory agent and/or anticoagulant.

Exemplary anti-inflammatory agents include NSAIDs (e.g. acetylsalicylic acid (aspirin), diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin, zomepirac, celecoxib, deracoxib, etoricoxib, mavacoxib or parecoxib), disease-modifying antirheumatic drugs (DMARDs) (e.g. methotrexate, leflunomide, sulfasalazine, hydroxychloroquinone, penicillamine, anatacept, baricitinib, cetolizumab, sarilumab, tocilizumab or tofacitinib), prednisone, methylprednisolone, dexamethasone, hydrocortisone, budesonide, prednisolone, etanercept, golimumab, infliximab, adalimumab, anakinra, rituximab, natalizumab and abatacept.

Representative anticoagulants include, but are not limited to, warfarin, heparin, fondaparinux, idraparinux, idrabiotaparinux, rivaroxaban, dabigatran, apixaban, edoxaban, betrixaban, letaxaban, eribaxaban, hirudin, lepirudin, bivalirudin, argatroban, dabigatran, ximelagatran, antithrombin, enoxaparin and dalteparin.

As previously described, the proteinaceous molecule may be compounded for convenient and effective administration in effective amounts with a suitable pharmaceutically acceptable carrier in dosage unit form. In some embodiments, a unit dosage form may comprise the proteinaceous molecule in an amount in the range of from about 0.25 μg to about 2000 mg. The proteinaceous molecule may be present in an amount of from about 0.25 μg to about 2000 mg/mL of carrier. In embodiments where the pharmaceutical composition comprises one or more additional active ingredients, the dosages are determined by reference to the usual dose and manner of administration of the said ingredients.

### 5. Methods of Use

The proteinaceous molecules of the invention have been found to inhibit FXIIa activity, with high potency and/or selectivity for FXIIa over one or more other serine proteases, such as trypsin. As such, the inventors have conceived that the proteinaceous molecules may be useful for treating or inhibiting the development of a condition associated with FXIIa activity, including thromboembolism-associated conditions such as acute coronary syndrome, stroke, deep vein thrombosis and pulmonary embolism, a thrombosis, a thrombosis-associated hematologic disorder, such as sickle cell disease or thrombophilia, or an inflammatory condition or a condition related to the kallikrein-kinin system, such as hereditary angioedema, multiple sclerosis, rheumatoid arthritis or lupus, or for treating or inhibiting thrombus and/or embolus formation. Accordingly, a proteinaceous molecule of the invention for use in therapy is contemplated.

In one aspect, there is provided a method of treating or inhibiting the development of a condition in which inhibiting FXIIa activity is associated with effective treatment or inhibition, comprising administering the proteinaceous molecule of the invention. Further provided is a use of a proteinaceous molecule of the invention for treating or inhibiting the development of a condition in which inhibiting FXIIa activity is associated with effective treatment or inhibition, a proteinaceous molecule of the invention for use in treating or inhibiting the development of a condition in which inhibiting FXIIa activity is associated with effective treatment or inhibition, and the use of a proteinaceous molecule of the invention in the manufacture of a medicament for treating or inhibiting the development of a condition in which inhibiting FXIIa activity is associated with effective treatment or inhibition.

In a related aspect, there is provided a method of treating or inhibiting the development of a condition in which antagonizing FXIIa stimulates or effects treatment or inhibition of the development of the condition, comprising administering the proteinaceous molecule of the invention. Also contemplated is a use of a proteinaceous molecule of the invention for treating or inhibiting the development of a condition in which antagonizing FXIIa stimulates or effects treatment or inhibition of the development of the condition, a proteinaceous molecule of the invention for use in treating or inhibiting the development of a condition in which antagonizing FXIIa stimulates or effects treatment or inhibition of the development of the condition and the use of a proteinaceous molecule of the invention in the manufacture of a medicament for treating or inhibiting the development of a condition in which antagonizing FXIIa stimulates or effects treatment or inhibition of the development of the condition.

FXIIa is well known in the art to be associated with a number of conditions, especially conditions associated with coagulation and thrombus or embolus formation, and inflammation due to its participation in the coagulation pathway (e.g. the intrinsic coagulation pathway) and kallikrein-kinin system.

As such, in some embodiments of any of the methods or uses described above, the condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, sickle cell disease, thrombophilia, and thrombosis resulting from a medical implant, device or extracorporeal circulation procedure in which blood is exposed to an artificial surface that promotes thrombosis.

The condition may also be an inflammatory condition or a condition related to the kallikrein-kinin system, such as hereditary angioedema, anaphylaxis, rheumatoid arthritis, a bacterial infection of the lung, a trypanosoma infection, hypotensive shock, pancreatitis, Chagas disease, articular gout, disseminated intravascular coagulation, sepsis, multiple sclerosis or lupus; especially hereditary angioedema.

In particular embodiments, the condition is an inflammatory condition, such as hereditary angioedema, anaphylaxis, rheumatoid arthritis, pancreatitis, sepsis, multiple sclerosis or lupus; especially hereditary angioedema.

The condition may also be related to angiogenesis or may be a condition associated with increased vascular permeability, such as progressive retinopathy, sight-threatening complication of retinopathy, macular edema, non-proliferative retinopathy, proliferative retinopathy, retinal edema, diabetic retinopathy, hypertensive retinopathy, and retinal trauma.

In another aspect, there is provided a method of treating or inhibiting the development of a thrombosis in a subject, comprising administering a proteinaceous molecule of the invention to the subject. Further provided is the use of a proteinaceous molecule of the invention for treating or inhibiting the development of a thrombosis in a subject, a proteinaceous molecule of the invention for use in treating or inhibiting the development of a thrombosis in a subject, and a use of a proteinaceous molecule of the invention in the manufacture of a medicament for treating or inhibiting the development of a thrombosis in a subject.

Also contemplated is the use of the proteinaceous molecule of the invention for inhibiting or reducing coagulation in a subject. As such, provided herein is a method of inhibiting coagulation in a subject, comprising administering a proteinaceous molecule of the invention to the subject, a proteinaceous molecule of the invention for use in inhibiting coagulation in a subject, and a use of a proteinaceous molecule of the invention in the manufacture of a medicament for inhibiting coagulation in a subject. Also provided is a proteinaceous molecule of the invention for use as an anticoagulant, and an anticoagulant comprising the proteinaceous molecule of the invention.

In particular embodiments, the subject is one who is experiencing coagulation at an elevated level compared to the level of coagulation in a healthy subject.

In some embodiments, the subject is one who has recently undergone a medical or surgical procedure, for example, within the previous seven days. The medical or surgical procedure may be any procedure which is associated with an increased risk of coagulation during or following the procedure. Exemplary procedures include, but are not limited to, cardiopulmonary bypass, percutaneous coronary intervention and hemodialysis. In alternative embodiments, the subject is one who is undergoing a medical or surgical procedure.

In another aspect, there is provided a method for inhibiting thrombus or embolus formation in a subject, comprising administering the proteinaceous molecule of the invention to the subject to thereby inhibit thrombus or embolus formation in the subject. Also contemplated is a use of a proteinaceous molecule of the invention for inhibiting thrombus or embolus formation in a subject, a proteinaceous molecule of the invention for use in inhibiting thrombus or embolus formation in a subject, and a use of a proteinaceous molecule of the invention in the manufacture of a medicament for inhibiting thrombus or embolus formation in a subject.

In some embodiments, the subject is one who has a thrombus or embolus, and/or is at increased risk of developing a thrombus or embolus.

In some embodiments, the subject may be suffering from a condition associated with thrombus or embolus formation, such as unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from a medical implant, device or extracorporeal circulation (ECMO, cardiopulmonary bypass) procedure in which blood is exposed to an artificial surface that promotes thrombosis.

The proteinaceous molecules of the invention are also useful for treating a subject suffering from a thrombus or embolus.

Accordingly, further contemplated, in another aspect, is a method of treating or inhibiting the development of a thromboembolism-associated condition in a subject, comprising administering the proteinaceous molecule of the invention to the subject. Also provided is a use of a proteinaceous molecule of the invention for treating or inhibiting the development of a thromboembolism-associated condition in a subject, a proteinaceous molecule of the invention for use in treating or inhibiting the development of a thromboembolism-associated condition in a subject, and a use of a proteinaceous molecule of the invention in the manufacture of a medicament for treating or inhibiting the development of a thromboembolism-associated condition in a subject.

Suitable thromboembolism-associated conditions include, for example, arterial cardiovascular thromboembolic disorders, venous cardiovascular or cerebrovascular thromboembolic disorders and thromboembolic disorders in the chambers of the heart or in the peripheral circulation. The thromboembolism-associated condition can also include specific disorders selected from, but not limited to, abdominal aortic aneurysm, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis and/or embolism, cerebral arterial thrombosis, cerebral embolism, kidney embolism, pulmonary embolism, and thrombosis resulting from medical implants, devices, extracorporeal circulation (ECMO, cardiopulmonary bypass) procedures in which blood is exposed to an artificial surface that promotes thrombosis. The medical implants or devices include, but are not limited to, prosthetic valves, artificial valves, indwelling catheters, stents, blood oxygenators, shunts, vascular access ports, ventricular assist devices and artificial hearts or heart chambers, and vessel grafts. The procedures include, but are not limited to, cardiopulmonary bypass, percutaneous coronary intervention, and hemodialysis. In particular embodiments, the disease or condition associated with thromboembolism is selected from acute coronary syndrome, stroke, deep vein thrombosis, and pulmonary embolism.

Further provided herein are methods for treating or inhibiting the development of a hematologic disorder (e.g. a thrombosis-associated hematologic disorder) in a subject. Accordingly, in another aspect, there is provided a method for treating or inhibiting the development of a thrombosis-associated hematologic disorder in a subject, comprising administering the proteinaceous molecule of the invention to the subject. Also provided is a use of a proteinaceous molecule of the invention for treating or inhibiting the development of a thrombosis-associated hematologic disorder in a subject, a proteinaceous molecule of the invention for use in treating or inhibiting the development of a thrombosis-associated hematologic disorder in a subject, and a use of a proteinaceous molecule of the invention in the manufacture of a medicament for treating or inhibiting the development of a thrombosis-associated hematologic disorder in a subject.

Non-limiting examples of hematologic disorders include sickle cell disease and thrombophilia.

In a further aspect, there is provided a method of treating an inflammatory condition in a subject, comprising administering a proteinaceous molecule of the invention to the subject. Also provided is a use of a proteinaceous molecule of the invention for treating an inflammatory condition in a subject; a proteinaceous molecule of the invention for use in treating an inflammatory condition in a subject; and a use of a proteinaceous molecule of the invention in the manufacture of a medicament for treating an inflammatory condition in a subject.

In some embodiments, the inflammatory condition is hereditary angioedema, anaphylaxis, rheumatoid arthritis, pancreatitis, sepsis, multiple sclerosis or lupus; especially hereditary angioedema. In particular embodiments, the inflammatory condition is associated with increased neutrophil activity.

In another aspect, there is provided a method of inhibiting or reducing an activity of FXIIa, comprising contacting FXIIa with a proteinaceous molecule of the invention, or a use of a proteinaceous molecule of the invention as an FXIIa inhibitor or antagonist. Also provided is a method of antagonizing FXIIa, comprising contacting FXIIa with a proteinaceous molecule of the invention.

The methods may inhibit one or more activities of FXIIa, including, but not limited to, enzymatic activity (e.g. proteolytic activity), factor XI activation, prekallikrein activation, plasminogen activation and a downstream activity thereof such as bradykinin release through the kallikrein-kinin system and thrombus formation through the coagulation system. In particular embodiments, the proteinaceous molecules of the invention inhibit the enzymatic activity of FXIIa and, consequently, inhibit factor XI activation and/or prekallikrein activation.

In particular embodiments of any one of the aspects described herein, FXIIa is α- or β-FXIIa, especially β-FXIIa, most especially human β-FXIIa.

The proteinaceous molecule of the invention may also be used as a coating on a medical device. Any medical device intended to be inserted into the human body may be suitable for such coating. Exemplary devices include, but are not limited to, a cardiopulmonary bypass machine, blood oxygenators including an extracorporeal membrane oxygenation (ECMO) system for oxygenation of blood, a device for assisted pumping of blood including a ventricular assist device, a blood dialysis device, a device for the extracorporeal filtration of blood, a repository for use in the collection of blood, a vascular access port, an indwelling catheter, a stent, a shunt, an artificial or prosthetic valve such as a heart valve, an artificial heart or heart chamber, and/or accessories for any one of said devices including tubing, cannulas, centrifugal pump, valve, port, and/or diverter.

The use of the proteinaceous molecule of the invention for inhibiting or reducing coagulation in a medical device is also encompassed herein. Suitable medical devices are discussed supra. In such uses, the proteinaceous molecule may be applied as a coating on the medical device, may be administered to a subject who is using or being treated with the medical device (such as a cardiopulmonary bypass machine or blood oxygenator including an ECMO system for oxygenation of blood), or may be delivered directly to or infused directly into the medical device (e.g. by injection or infusion into the tubing or tubing associated with the device).

Any one of the methods and uses described above may involve administration of an effective amount of the proteinaceous molecule of the invention as described in Section 4 supra. The proteinaceous molecule of the invention may be administered via any suitable route of administration, such as oral, rectal, topical, intranasal, intraocular, transmucosal, intestinal, enteral, intramuscular, subcutaneous, intramedullary, intrathecal, intraventricular, intracerebral, intravaginal, intravesical, intravenous or intraperitoneal administration. In particular embodiments, the proteinaceous molecule is administered via oral or intravenous administration.

The dosage and frequency will depend on the subject, the condition, disease or disorder to be treated and the route of administration. A skilled person will readily be able to determine suitable dosages and frequency of such dosages. For example, the proteinaceous molecule may be administered in an amount in the range of from about 0.25 μg to about 2000 mg, and may be administered at a frequency of, for example, once daily, or twice or three times daily. The treatment may be continued for multiple days, weeks, months or years. In embodiments where the pharmaceutical composition comprises one or more additional active ingredients, the dosages and frequency of administration are determined by reference to the usual dose and manner of administration of the said ingredients.

Any one of the methods or uses described above may, in some embodiments, involve the administration of one or more further active agents as described in Section 4 supra, such as an anti-inflammatory agent or an anticoagulant.

A skilled person will be well aware of suitable assays used to evaluate the antagonism of FXIIa and/or an inhibition or reduction of FXIIa activity or function. For example, the method may include contacting FXIIa (e.g. immobilized FXIIa) with a proteinaceous molecule and assessing the binding affinity or the inhibition of the enzymatic activity, e.g. proteolytic activity. Alternatively, the method may include screening for the inhibition of the activity, presence or expression of a downstream cellular target or product, or a downstream effect, such as factor XI activation (e.g. presence of FXIa), prekallikrein activation (e.g. presence of kallikrein), or clotting time. Detecting such inhibition may be achieved utilizing techniques including, but not limited to, ELISA, a binding assay (e.g. a radioligand binding assay or fluorescence binding assay), surface plasmon resonance, immunofluorescence, Western blots, immunoprecipitation, immunostaining, scintillation proximity assays, competitive inhibition assays, a colorimetric assay and coagulation assays as described further in the examples herein. In particular embodiments, affinity is assessed in a HBS-EP+ buffer, comprising 10 mM HEPES, 150 mM NaCl, 3 mM EDTA and 0.05% (v/v) surfactant P20, at pH 7.4. In some embodiments, the temperature is in the range of from about 15° C. to about 25° C. (and all integer degrees therebetween), especially about 20° C. Commercially available kits and/or products may also be used, such as Factor XIIa Activity Kit (Colorimetric) (Catalogue No. LS-K776; LSBio, Seattle, USA) or the Factor XII/XIIa Assay Kit (Catalogue No. ab241041; Abcam plc, Waltham, USA).

### 6. Methods of Identification

The inventors have further conceived that disulfide rich peptides, such as peptides with at least six cysteine residues and three disulfide bonds, can be identified using in vitro mRNA display techniques involving a prokaryotic translation system.

Accordingly, in another aspect, there is provided an in vitro method for identifying a disulfide rich peptide which binds to a target substance comprising:
a) preparing an mRNA library based on a disulfide rich peptide scaffold;
b) ligating mRNA in the library to puromycin to form mRNA-puromycin conjugates;
c) translating the mRNA-puromycin conjugates using a prokaryotic translation system to produce mRNA-puromycin-peptide conjugates;
d) reverse transcribing the conjugates to form mRNA:cDNA-puromycin-peptide conjugates;
e) performing affinity selection against the target substance to select for mRNA:cDNA-puromycin-peptide conjugates that bind to the target substance;
f) performing nucleic acid amplification on the cDNA of the selected mRNA:cDNA-puromycin-peptide conjugates to generate an enriched cDNA library; and
g) sequencing the enriched cDNA library to identify a disulfide rich peptide which binds to the target substance.

While the disulfide rich peptide may be any peptide comprising at least four cysteine residues, in particular embodiments, the disulfide rich peptide contains at least six cysteine residues, especially six cysteine residues. In such embodiments, the cysteine residues are bound in pairs to form at least three disulfide bonds, especially three disulfide bonds. In some embodiments, the disulfide rich peptide contains a cystine knot motif.

For example, a suitable disulfide rich peptide is a peptide comprising, consisting or consisting essentially of an amino acid sequence represented by Formula X:

C(X^{I})ₐC(X^{II})_{b}C(X^{III})_{c}C(X^{IV})_{d}C(X^{V})ₑC (X)

wherein each X^{I}, X^{II}, X^{III}, X^{IV} and X^{V} are independently selected from any amino acid residue;
a, b, c, d and e represent the number of amino acids in each respective sequence, and each of a to e are independently selected from about 1 to about 10 (and all integers in between).

In some embodiments, the disulfide rich peptide and/or disulfide rich peptide template is a cyclic peptide comprising, consisting or consisting essentially of an amino acid sequence represented by Formula XI:

[C(X^{I})ₐC(X^{II})_{b}C(X^{III})_{c}C(X^{IV})_{d}C(X^{V})ₑC(X^{VI})f] (XI)

wherein each X^{I}, X^{II}, X^{III}, X^{IV}, X^{V} and X^{VI} are independently selected from any amino acid residue; a, b, c, d, e and f represent the number of amino acids in each respective sequence, and each of a to f are independently selected from about 1 to about 10 (and all integers in between).

In such embodiments, the peptide is cyclized using N-to-C-cyclization, for example via an amide bond.

In some embodiments of Formula X or XI, a is from about 3 to about 6, especially 6; b is from about 3 to about 5, especially 5; c is from about 2 to about 7, especially 3; d is from about 1 to about 3, especially 1; and e is from about 3 to about 6; especially 5. In some embodiments, a is from about 3 to about 6, especially 6; b is from about 3 to about 5, especially 5; c is from about 2 to about 7, especially 3; d is from about 1 to about 3, especially 1; e is from about 3 to about 6; especially 5; and f is from about 2 to about 9, especially from about 2 to about 8, more especially 8.

In some embodiments of Formula X or XI, a is 6, b is 5, c is 3, d is 1 and e is 5. In some embodiments, a is 6, b is 5, c is 3, d is 1, e is 5 and f is from about 2 to about 8, especially 8.

In some embodiments, the disulfide rich peptide scaffold is a cyclotide, especially a peptide comprising the amino acid sequence of SEQ ID NO: 1, 43 or 44.

The disulfide rich peptide, in some embodiments, has greater affinity for binding to the target substance than the disulfide rich peptide scaffold. For example, in some embodiments, the disulfide rich peptide has at least about 2-fold greater binding affinity for the target substance than the disulfide rich peptide scaffold. In some embodiments, the disulfide rich peptide has at least about 5-fold, 10-fold, 20-fold, 50-fold or 100-fold greater binding affinity for the target substance than the disulfide rich peptide scaffold.

The disulfide rich peptide may have greater selectivity for the target substance than the disulfide rich peptide scaffold. For example, in some embodiments, the disulfide rich peptide has at least about 2-fold greater selectivity for the target substance than the disulfide rich peptide scaffold. In some embodiments, the disulfide rich peptide has at least about 5-fold, 10-fold, 20-fold, 50-fold or 100-fold greater selectivity for the target substance than the disulfide rich peptide scaffold. The disulfide rich peptide may be more selective for the target substance than a related molecule, for example, a protein from the same family when the target substance is a protein (e.g. selectivity for one serine protease over at least one other serine protease).

The target substance may be any substance for which binding of a disulfide rich peptide is desired, such as a substance in which binding of a disulfide rich peptide results in a therapeutic effect. A skilled person will be aware of suitable target substances. For example, in some embodiments, the target substance is a protein, such as a receptor (e.g. a G-protein coupled receptor, nuclear hormone receptor, growth factor receptor such as epidermal growth factor receptor), ion channel (e.g. ligand-gated ion channel such as glutamate receptors, GABA receptors, P2X receptor or 5-HT3 receptor; or voltage-gated ion channel such as calcium, potassium, chloride, proton and sodium channels), enzyme (including a kinase, protease e.g. a serine protease, esterase or phosphatase) or membrane transport protein; especially a receptor, ion channel or enzyme. In particular embodiments, the target substance is a protease, such as a serine protease, for example FXIIa.

The preparation of an mRNA library may be achieved using techniques known in the art. For example, oligonucleotides may be prepared based on the sequence of the disulfide rich peptide scaffold, but containing residues encoded by an NNK codon (wherein N=A, T, G and C, and K=G and T) in positions with desired variability. The encoded sequences may further contain a formyl-Met residue for translation initiation at the N-terminus and a C-terminal spacer for attachment to puromycin. For example, the C-terminal spacer may be any sequence that provides an appropriate distance for the efficient incorporation of puromycin into the ribosome and/or a distance which minimizes the effect of the mRNA-puromycin conjugate on binding of the translated peptide to the target substance. The C-terminal spacer may be, for example, an amino acid sequence comprising about 1 to about 20 amino acid residues (and all integer amino acid residues therebetween); especially about 1 to about 10 amino acid residues; more especially about 2 to about 6 amino acid residues. In some embodiments, the C-terminal spacer is an amino acid sequence comprising about 1, 2, 3, 4, 5, 6, 7, 8, 9 or 10 amino acid residues; especially about 2, 3, 4, 5 or 6 amino acid residues; most especially about 2 or about 6 amino acid residues. The amino acid residues may be any amino acid residues, especially Gly, Ser, Asn, Asp and/or Gln. In particular embodiments, the amino acid residues comprise Gly and Ser residues. Exemplary C-terminal spacers include one of the following amino acid sequences: GS, GSGSGS, SGSGSG, GQGQGQ, SSGSSG, SGGSGG, SDSDSD or SSNSSN; especially GS or GSGSGS. Suitable C-terminal spacers may be encoded by a polynucleotide sequence comprising from about 3 to about 60 nucleotides (and all integer nucleotides therebetween); especially about 3 to about 30 nucleotides; more especially about 6 to about 18 nucleotides. In some embodiments, the polynucleotide sequence comprises about 3, 6, 9, 12, 15, 18, 21, 24, 27 or 30 nucleotides; especially about 6, 9, 12, 15 or 18 nucleotides; more especially about 6 or about 18 nucleotides.

A DNA library may then be constructed using a nucleic acid amplification technique, such as the polymerase chain reaction (PCR), ligase chain reaction, transcription-mediated amplification, rolling circle amplification, and the like, especially PCR. For example, the PCR reaction may be a two-step PCR reaction using a DNA polymerase, with the first step extending two pieces of oligonucleotides containing the peptide-coding region and a second amplification step adding the upstream T7 promoter, GGG triplet, epsilon sequence and ribosome binding sequence, and the downstream puromycin binding sequence. Exemplary sequences are described in Table 4. Exemplary conditions for the PCR reaction are as described in Table 15. PCR products may then be extracted (using, e.g., phenol/chloroform), precipitated (e.g. using ethanol), dissolved in an aqueous solution and used for in vitro transcription.

Techniques for transcription of a DNA library are well known in the art. Transcription may be achieved using a buffer solution comprising an RNA polymerase, such as a T7 RNA polymerase. Suitable buffer solutions may comprise, for example, Tris-HCl, spermidine, Triton X-100, DTT, MgCl₂, NTPs and KOH, together with the DNA and the RNA polymerase. The buffer, DNA and RNA polymerase may be incubated at a temperature in the range of from about 20 to about 40° C. (and all integer degrees therebetween), especially about 37° C., for a time period suitable to enable transcription to occur, such as a time period in the range of from about 10 hrs to about 20 hrs (and all integer hrs therebetween), especially about 16 hrs. The mRNA transcripts are then precipitated and purified using, for example, NaCl and isopropanol for precipitation, and polyacrylamide gel electrophoresis for purification.

Following transcription, the mRNA library is then ligated to puromycin via a covalent bond to form mRNA-puromycin conjugates. The mRNA library may be directly attached to puromycin, or may be indirectly attached to puromycin via a linker, such as a nucleic acid linker (e.g. DNA or RNA, especially DNA). In particular embodiments, the linker is a polynucleotide, or a polynucleotide-PEG conjugate. The linker may be incorporated into the mRNA sequence during preparation of the library (e.g. via a C-terminal spacer in the encoded peptide sequence as discussed supra), the 5′ end of the linker may be attached to the 3′ end of the mRNA via a covalent bond prior to ligation with puromycin, or the 3′ end of the linker may be attached to puromycin via a covalent bond prior to ligation with the mRNA. Suitable linkers include any moiety that can provide a suitable distance for efficient incorporation of puromycin into the ribosome. In particular embodiments, the linker comprises a nucleic acid, such as a nucleic acid comprising about 1 to about 60 nucleotides (and all integer nucleotides therebetween); especially about 1 to about 30 nucleotides; more especially about 6, 7, 8, 9, 10, 11, 12, 13, 14, 15, 16, 17, 18, 19 or 20 nucleotides. The linker may also comprise a non-nucleic acid section, such as a polyethylene glycol (e.g. PEG18), to form a polynucleotide-PEG conjugate. In such embodiments, the linker is preferably of the formula: nucleic acid-PEG-nucleic acid, such as CTCCCGCCCCCCGTCC-(PEG18)5-CC (e.g. the linker in Table 4). Alternatively, the linker may be a non-nucleic acid moiety or part nucleic acid moiety (e.g. a nucleic acid-small molecule conjugate), with a phosphate group or nucleotide at the 5′ end of the moiety, and a group suitable for attachment to puromycin at the 3′ end of the moiety, such as a nucleotide or a hydroxyl group. Covalent bond formation may be achieved using techniques standard in the art, such as using RNA ligase, DNA ligase or standard organic chemistry techniques.

Ligation to puromycin may be achieved using techniques standard in the art, for example, by incubating the mRNA with puromycin (refer to, e.g., Table 4) and T4 RNA ligase under conditions suitable for ligation, e.g. a temperature in the range of from of about 20° C. to about 30° C., especially about 25° C., for a time period in the range of from about 20 mins to about 1 hr (and all integer mins therebetween), especially about 30 mins.

The mRNA library containing mRNA-puromycin conjugates is then translated using a prokaryotic translation system to produce mRNA-puromycin-peptide conjugates. In preferred embodiments, the prokaryotic translation system is a cell-free system. While the use of any prokaryotic translation system is contemplated, in particular embodiments, the prokaryotic translation system is an E. coli translation system (i.e. the prokaryote is E. coli).

In particular embodiments, the prokaryotic translation system does not comprise release factor 1 (RF1).

The prokaryotic translation system contains components which will enable translation of an mRNA sequence into a protein. For example, the translation system, in some embodiments, comprises tRNAs, initiation factors, elongation factors, release factors, T7 RNA polymerase, nucleoside triphosphates, aminoacyl-tRNA synthetases (ARS), ribosomes and the 20 natural amino acids.

In particular embodiments, the ribosomes, tRNAs, initiation factors, elongation factors and/or release factors are prokaryotic, especially from E. coli. In specific embodiments, the translation system comprises E. coli ribosomes, E. coli tRNAs, E. coli initiation factors, E. coli elongation factors and/or E. coli release factors.

In some embodiments, the translation system comprises ribosome, initiation factor 1 (IF1), initiation factor 2 (IF2), initiation factor 3 (IF3), elongation factor G (EF-G), elongation factor thermo unstable (EF-Tu), elongation factor thermo stable (EF-Ts), release factor 2 (RF2), release factor 3 (RF3), ribosome release factor (RRF), alanyl-tRNA synthetase (AlaRS), arginyl-tRNA synthetase (ArgRS), asparaginyl-tRNA synthetase (AsnRS), aspartyl-tRNA synthetase (AspRS), cysteinyl-tRNA synthetase (CysRS), glutamyl-tRNA synthetase (GluRS), glutaminyl-tRNA synthetase (GlnRS), glycyl-tRNA synthetase (GlyRS), histidyl-tRNA synthetase (HisRS), isoleucyl-tRNA synthetase (IleRS), leucyl-tRNA synthetase (LeuRS), lysyl-tRNA synthetase (LysRS), methionyl-tRNA synthetase (MetRS), phenylalanyl-tRNA synthetase (PheRS), prolyl-tRNA synthetase (ProRS), seryl-tRNA synthetase (SerRS), threonyl-tRNA synthetase (ThrRS), tryptophanyl-tRNA synthetase (TrpRS), tyrosyl-tRNA synthetase (TyrRS), valyl-tRNA synthetase (VaIRS), methionyl-tRNA formyltransferase (MTF), T7 RNA polymerase, tRNA, adenosine triphosphate (ATP), guanosine triphosphate (GTP), cytidine triphosphate (CTP), uridine triphosphate (UTP) and amino acids.

In particular embodiments, the translation system comprises E. coli ribosome, IF1, IF2, IF3, EF-G, EF-Tu, EF-Ts, RF2, RF3, RRF, AlaRS, ArgRS, AsnRS, AspRS, CysRS, GluRS, GlnRS, GlyRS, HisRS, IleRS, LeuRS, LysRS, MetRS, PheRS, ProRS, SerRS, ThrRS, TrpRS, TyrRS, VaIRS, MTF, T7 RNA polymerase, E. coli total tRNA, ATP, GTP, CTP, UTP and the 20 natural amino acids.

In particular embodiments, the translation system further comprises inorganic pyrophosphatase, nucleoside diphosphate kinase, creatine phosphate, 10-formyl-5,6,7,8-tetrahydrofolic acid, spermidine, dithiothreitol (DTT), potassium acetate, magnesium acetate, HEPES-KOH buffer, myokinase and creatine kinase. In some embodiments, the translation system further comprises an aqueous solution, such as water.

A skilled person will be well aware of suitable amounts of each component of the translation system. For example, in specific embodiments, the translation system comprises about 50 mM HEPES-KOH buffer (about pH 7.6), about 100 mM potassium acetate, about 12.3 mM magnesium acetate, about 2 mM ATP, about 2 mM GTP, about 1 mM CTP, about 1 mM UTP, about 20 mM creatine phosphate, about 2 mM spermidine, about 1 mM dithiothreitol, about 100 μM 10-formyl-5,6,7,8-tetrahydrofolic acid, about 1.5 mg/mL E. coli total tRNA, about 1.2 μM E. coli ribosome, about 0.6 μM methionyl-tRNA formyltransferase, about 2.7 μM IF1, about 0.4 μM IF2, about 1.5 μM IF3, about 0.26 μM EF-G, about 10 μM EF-Tu/EF-Ts complex, about 0.25 μM RF2, about 0.17 μM RF3, about 0.5 μM RRF, about 4 μg/mL creatine kinase, about 3 μg/mL myokinase, about 0.1 μM inorganic pyrophosphatase, about 0.1 μM nucleotide diphosphate kinase, about 0.1 μM T7 RNA polymerase, about 0.73 μM AlaRS, about 0.03 μM ArgRS, about 0.38 μM AsnRS, about 0.13 μM AspRS, about 0.02 μM CysRS, about 0.06 μM GlnRS, about 0.23 μM GluRS, about 0.09 μM GlyRS, about 0.02 μM HisRS, about 0.4 μM IleRS, about 0.04 μM LeuRS, about 0.11 μM LysRS, about 0.03 μM MetRS, about 0.68 μM PheRS, about 0.16 μM ProRS, about 0.04 μM SerRS, about 0.09 μM ThrRS, about 0.03 μM TrpRS, about 0.02 μM TyrRS and about 0.02 μM VaIRS, about 0.5 mM each of Ala, Arg, Asn, Asp, Cys, Gln, Glu, Gly, His, Ile, Leu, Lys, Met, Phe, Pro, Ser, Thr Trp, Tyr and Val, and about 1.2 μM of the mRNA-puromycin conjugates.

In particular embodiments, multiple rounds of selection may be performed, for example two, three or four rounds of selection, especially four rounds of selection. For example, prior to step g) (sequencing of the enriched cDNA library), an mRNA library is prepared based on the enriched cDNA library produced in step f). Steps b) to f) are then repeated using this mRNA library. In such embodiments, the method may further comprise between steps f) and g):
f1) preparing an mRNA library from the enriched cDNA library of step f);
f2) ligating mRNA in the library to puromycin to form mRNA-puromycin conjugates;
f3) translating the mRNA-puromycin conjugates using a prokaryotic translation system to produce mRNA-puromycin-peptide conjugates;
f4) reverse transcribing the conjugates to form mRNA:cDNA-puromycin-peptide conjugates;
f5) performing affinity selection against the target substance to select for mRNA:cDNA-puromycin-peptide conjugates that bind to the target substance; and
f6) performing nucleic acid amplification on the cDNA of the selected mRNA:cDNA-puromycin-peptide-conjugates to generate an enriched cDNA library.

This sequence may be repeated one or more further times using the enriched cDNA library of each round. For example, steps f1) to f6) may be repeated one, two or three times using the resulting enriched cDNA library of each round.

In some embodiments, translation is performed at a temperature in the range of from about 20 to about 40° C. (and all integer degrees therebetween), especially about 37° C., and for a time period in the range of from about 20 mins to about 1 hr (and all integer mins therebetween), especially about 30 mins to about 45 mins. In some embodiments, the translation of step c) is performed at 37° C. for about 45 mins and the translation of the following rounds (e.g. steps f3) and f9)) are performed at about 37° C. for about 30 mins. In some embodiments, the translation mixture may then be incubated at a temperature in the range of from of about 20° C. to about 30° C., especially about 25° C., for a time period in the range of from about 10 to about 20 mins (and all integer mins therebetween), especially about 12 mins. Following incubation, the ribosomes are dissociated from the mRNA-puromycin-peptide conjugates using, for example, ethylene diamine tetraacetic acid (EDTA) (e.g. 100 mM), and the mixture may be incubated for a time period suitable for such dissociation, such as at a temperature in the range of from about 20 to about 40° C. (and all integer degrees therebetween), especially about 37° C., and for a time period in the range of from about 20 mins to about 1 hr (and all integer mins therebetween), especially about 30 mins.

Reverse transcription (e.g. step d)) may be performed using methods well known in the art. For example, in some embodiments, reverse transcription is conducted at a temperature in the range of from about 30° C. to about 60° C. (and all integer degrees therebetween), especially about 42° C. for a period of time in the range of from about 10 mins to about 20 mins (and all integer mins therebetween), especially about 15 mins. A skilled person will be well aware of suitable primers and reverse transcriptases that may be used. In particular embodiments, the primer is a CGS3an13.R22 primer (refer to Table 4) and Moloney Murine Leukemia Virus (M-MLV) reverse transcriptase, which is substantially lacking RNase H activity (e.g. Catalogue No. M1701, Promega Corporation, Madison, USA).

Affinity selection may be conducted using techniques known in the art, and will depend on the nature and identity of the target substance. Generally, affinity selection comprises incubating the mRNA:cDNA-puromycin-peptide conjugates with the target substance to enable binding of the conjugate to the target substance and separating the bound conjugates from the unbound conjugates. The bound conjugates are then separated from the target substance and the cDNA sequence of the bound conjugates are subsequently enriched, for example using a nucleic acid amplification technique, such as PCR, in step f), and either sequenced or used for mRNA library generation for further selection rounds. In particular embodiments, the target substance (e.g. a protein) is immobilized on a solid support, such as a bead (e.g. a magnetic bead comprising streptavidin), using, for example, a biotin-conjugated target, and incubated with the mRNA:cDNA-puromycin-peptide conjugates for a time period suitable to enable binding. In some embodiments, the target substance and the mRNA:cDNA-puromycin-peptide conjugates are incubated for a time period in the range of from about 15 mins to 1 hr (and all integer mins therebetween), especially about 30 mins, at a temperature in the range of from about 2° C. to about 20° C. (and all integer degrees therebetween), especially about 4° C. Following incubation, the target substance, such as the immobilized target substance is washed to remove unbound mRNA:cDNA-puromycin-peptide conjugates, e.g. in a buffer such as phosphate buffered saline in the presence of a detergent (e.g. 0.05% Tween-20). The cDNA of the bound mRNA:cDNA-puromycin-peptide conjugates is then separated from the immobilized target substance using techniques known in the art. For example, bound conjugates may be removed by heating to a temperature in the range of from about 8° C. to about 100° C. (and all integer degrees therebetween), especially about 95° C., in a suitable buffer, such as the buffer in which PCR is to be conducted. For example, the buffer may comprise Tris-HCl, KCl, Triton X-100, dNTP and MgCl₂. Suitable primers, such as T7g10M.F46 and CGS3an13.R22 (refer to Table 4) may also be present in the buffer.

Following separation from the immobilized target substance, the cDNA of the selected mRNA:cDNA-puromycin-peptide conjugates is then amplified using nucleic acid amplification (e.g. PCR). In particular embodiments, the nucleic acid amplification is PCR. Suitable buffers and protocols for performing PCR are well known in the art. For example, in some embodiments, the PCR buffer comprises Tris-HCl, KCl, Triton X-100, dNTP, MgCl₂, together with suitable primers, such as T7g10M.F46 and CGS3an13.R22 (refer to Table 4). The protocol may comprise, for example, the protocol outlined in Table 15.

Following enrichment, the DNA library may be used for further rounds of selection or may be sequenced to determine the content of the library. Sequencing may be performed using techniques known in the art, such as next-generation sequencing, to identify the content of the library and the corresponding sequences of the disulfide rich peptides which bind to the target substance.

Further provided is a disulfide rich peptide identified using the in vitro methods of the invention.

The use of a disulfide rich peptide which binds to a target substance that is identified using the in vitro methods of the invention for therapy and for the treatment of a condition, disease or disorder is contemplated, such as one or more of the conditions, diseases or disorders described in Section 5 supra.

In order that the invention may be readily understood and put into practical effect, particular preferred embodiments will now be described by way of the following non-limiting examples.

### EXAMPLES

### Example 1—Affinity Selection of an MCoTI-II-Based Peptide Library Against FXIIa

mRNA display requires the C-terminal fusion of peptides to their cognate mRNAs, generally making it challenging to display head-to-tail cyclic peptides. Although the cystine knot scaffold of MCoTI-II (refer to Table 5) bears a head-to-tail cyclized structure, it adopts its bioactive conformation with three disulfide bonds even when linearized by breaking the cyclic backbone in loop 6. This property of MCoTI-II provides a means of fusing to cognate mRNA via the C-terminal region. Furthermore, several homologous acyclic cystine knot peptides exist in nature that have similar folds and inhibitory potency to trypsin-inhibiting cyclotides. Thus, a backbone-acyclic library containing semi-randomized MCoTI-II analogues for mRNA display was designed.

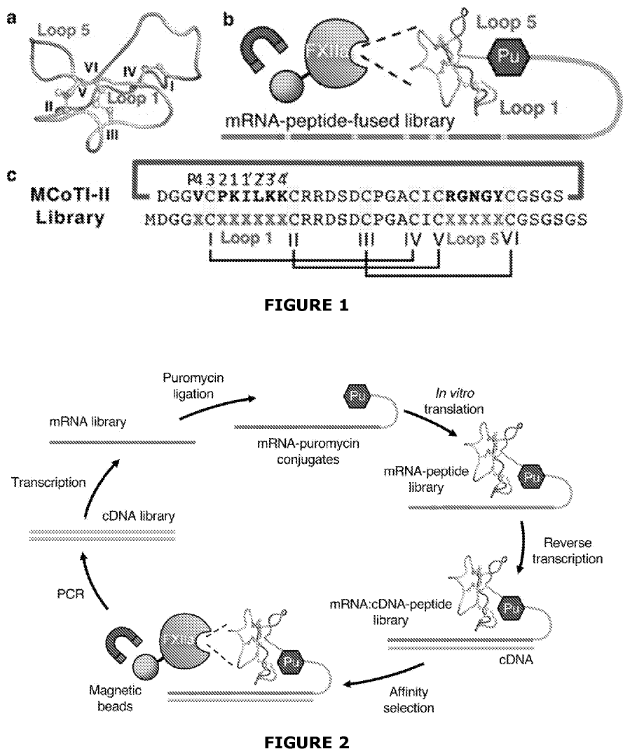

To assess the feasibility of such an approach, a peptide corresponding to a backbone-acyclic version of native MCoTI-II was translated using the FIT system, as previously described in Goto et al. (2011) Nat Protoc, 6: 779-790, the entire contents of which are incorporated by reference. This sequence was designed such that the normally head-to-tail cyclic MCoTI-II scaffold was split between the S and D residues in loop 6, with an N-terminal formyl-M residue added for translation initiation and a G-S dipeptide embedded in the C-terminus as a spacer for linkage to the cognate mRNA template via puromycin (refer to FIG. 1). LC-MS analysis demonstrated that this in vitro translated acyclic peptide adopted a single conformation with a similar retention time to synthetic (without N-formyl-M or C-terminal G-S dipeptide) backbone cyclic and acyclic (linearized as above through splitting the S-D peptide bond in loop 6) MCoTI-II, indicating that the correct cystine knot topology could be achieved in this system.

An MCoTI-II-based library was constructed and screened to identify variants bearing potent binding activity against FXIIa. A semi-randomized peptide library was generated based on the linearized and translatable MCoTI-II scaffold described above, such that the residues predicted to interact with trypsin-like proteases (all of loops 1 and 5, and a V residue in loop 6) were randomized to allow the occurrence of any of the 20 canonical amino acids at these 12 positions (refer to FIG. 1C). These positions included all eight residues flanking the scissile bond in MCoTI-II (the P4-P1 and P1′-P4′ sites), with the exception of the C residue at the P3 site, which was fixed to allow formation of the native cystine knot.

Following a standard selection protocol, the DNA encoding this library was assembled from degenerate oligonucleotides (refer to Table 4), transcribed into mRNA, ligated to a puromycin-linked oligonucleotide and translated in vitro to produce a library of mRNA-peptide fusion molecules. Based on the maximum amount of ribosome in the in vitro translation cocktail, the theoretical diversity of the mRNA-peptide fusion library was more than 10¹⁴ variants. However, the diversity was likely decreased during mRNA display processes (e.g. mRNA library gel purification and puromycin ligation) or due to the possible formation of misfolded variants. Therefore, it was conservatively estimated that the mRNA-peptide fusion library comprised in excess of 10¹² variants. This complexity of sequence space that can be explored by affinity selection is far larger than other approaches. The library was screened for affinity to biotinylated FXIIa immobilized on streptavidin-coated magnetic beads. After four rounds of selection (refer to FIG. 2), next-generation sequencing (NGS) was performed to identify clonal sequences within the enriched cDNA library after the third and fourth rounds. The 19 most abundant sequences exhibited high clonal convergence, considering each sequence was enriched from a single clone in the original library.

TABLE 4
SEQUENCES OF OLIGONUCLEOTIDES USED FOR LIBRARY ASSEMBLY, MRNA DISPLAY AND IN
VITRO TRANSLATION OF MCoTI-II
Oligonucleotides for library assembly and mRNA display

| | |
|---|---|
| MCoTI-II- | CTTTAAGAAGGAGATATACATATGGATGGTGGCNNKTGCNNKNNKNNKNNKNNKN |
| NNK7.F80 | NKTGCCGTCGTGATTCTGACTGCCC [SEQ ID NO: 100] |
| MCoTI-II- | GCCCCCCGTCCTAGCTGCCCGAACCTGACCCGCAMNNMNNMNNMNNMNNGCAA |
| NNK5.R82 | ATGCACGCACCGGGGCAGTCAGAATCACG [SEQ ID NO: 101] |
| T7g10M.F4 | TAATACGACTCACTATAGGGTTAACTTTAAGAAGGAGATATACATA [SEQ ID NO: |
| 6 | 102] |
| CGS3an13. | TTTCCGCCCCCCGTCCTAGCTG [SEQ ID NO: 103] |
| R22 | |
| Puromycin | CTCCCGCCCCCCGTCC-(PEG18)5-CC-Pu [SEQ ID NO: 104] |
| linker | |

Oligonucleotides for in vitro translation of MCoTI-II with the library design

| | |
|---|---|
| MCoTI-II- | CTTTAAGAAGGAGATATACATATGGATGGTGGCGTATGCCCCAAAATTCTGAAGAA |
| native.F78 | GTGCCGTCGTGATTCTGACTGC [SEQ ID NO: 105] |
| MCoTI-II- | GCCCCCCGTCCTAGCTGCCCGAACCTGACCCGCAATATCCGTTTCCACGGCAAAT |
| native.R87 | GCACGCACCGGGGCAGTCAGAATCACGACGGC [SEQ ID NO: 106] |

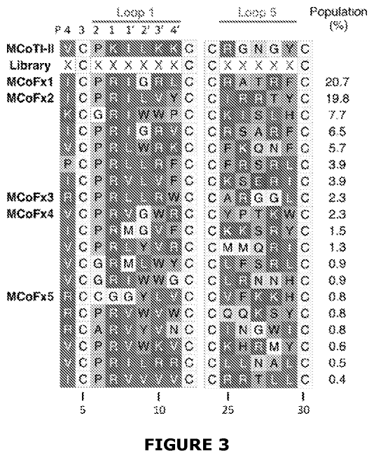

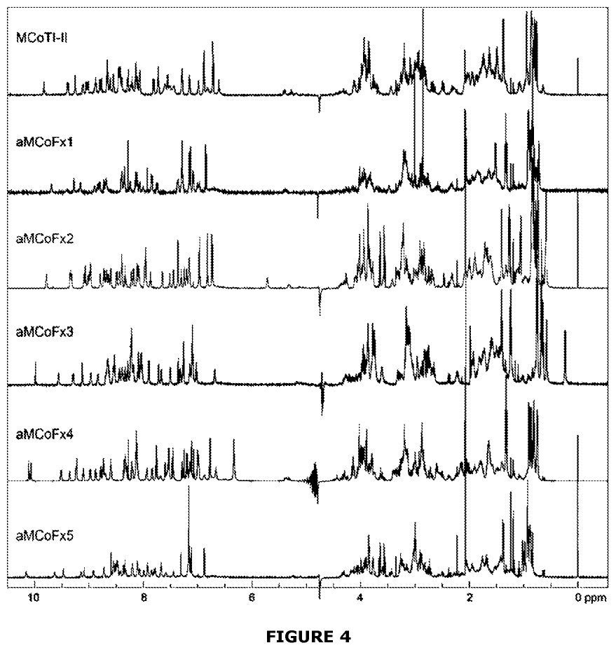

Strikingly, the 19 most abundant sequences exhibited sequence homology to MCoTI-II near the scissile bond in loop 1 (refer to FIG. 3). Hydrophobic or basic residues were preferred at the P4 site, and the P residue at position 6 (P6) in the P2 site was highly conserved (identical to MCoTI-II). Moreover, substitutions with R (R7) and a hydrophobic residue (I/L/V8) at P1 and P1′ sites, respectively, were frequently observed in the selected sequences.

In contrast to the conservation observed for the P4, P2, P1 and P1′ sites, residues at the P2′-P4′ sites were less conserved, but some preference trends were observed (refer to FIG. 3). Whereas MCoTI-II has consecutive K10 and K11 at the P3′ and P4′ sites, respectively, the affinity-selected peptides had a combination of hydrophobic residues (V, L, F, W or P) or a combination of a hydrophobic residues and a basic residue (R or K). This trend suggests that strong electrostatic interactions at these sites are not crucial for FXIIa binding. Since both R and K residues also possess a long hydrocarbon chain with a basic head group, the aliphatic moiety on the side chain could additionally facilitate hydrophobic interactions with the FXIIa surface. In contrast to loop 1, alignment of the loop 5 sequences showed little conservation, although one or two R or K residues were generally observed (refer to FIG. 3). This suggests that the basic residues possibly contribute to an electrostatic interaction with FXIIa and/or the peptide itself to improve its structural rigidity. An unusual sequence, MCoFx5, contained an additional C6 and a G7 at P2 and P1 sites, replacing the conserved P6 and R7 residues, respectively.

### Example 2—Activity of Selected FXIIa-Binding Peptides

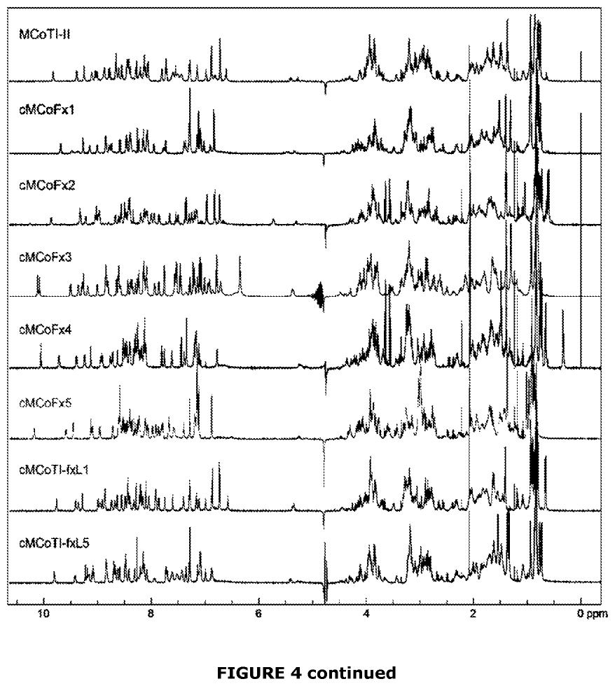

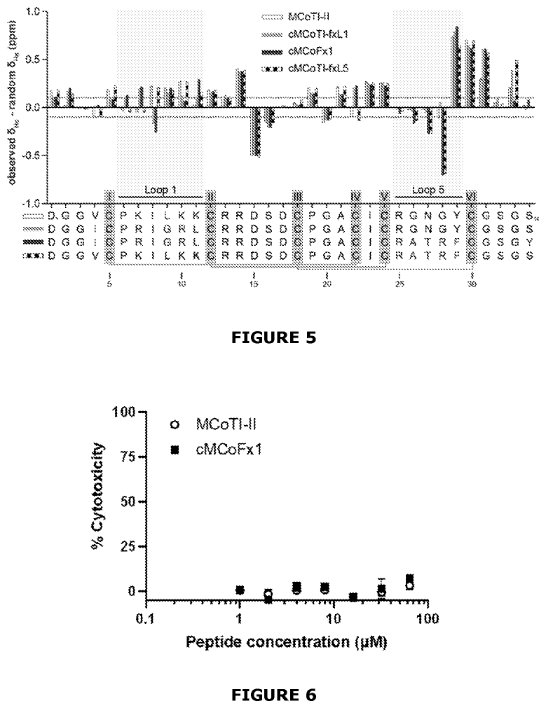

Five peptides (designated MCoFx1-5; refer to Table 5) from the top 19 selected sequences of Example 1 were synthesized using solid-phase peptide synthesis and were characterised. Both backbone-acyclic (as selected during display screening) and cyclic forms of each sequence were synthesized to determine the effect of backbone cyclization on FXIIa binding and inhibitory activity. The synthetic peptides were purified by RP-HPLC and characterized by analytical HPLC and MALDI-TOF mass spectroscopy (refer to Table 6). Conformations of each peptide were assessed by ¹H-NMR spectroscopy, which showed comparable peak patterns to those of MCoTI-II (refer to FIG. 4). Moreover, the α-proton NMR secondary chemical shifts of cMCoFx1 (refer to FIG. 5) indicated that cMCoFx1 adopted the native-like conformation of MCoTI-II. Some differences in chemical shifts were observed in loops 1 and 5, corresponding to sequence divergence from MCoTI-II.

TABLE 5
SEQUENCES OF FXIIA-BINDING PEPTIDES VARIANTS

| | | SEQ ID |
|---|---|---|
| Peptide | Sequence | NO |
| MCoTI-II | [DGGVCPKILKKCRRDSDCPGACICRGNGYCGSGS] | 71 |
| MCoFx1 | NH₂-DGGICPRIGRLCRRDSDCPGACICRATRFCGSGY-OH | 72 |
| acyclic | | |
| MCoFx1 | [DGGICPRIGRLCRRDSDCPGACICRATRFCGSGY] | 73 |
| cyclic | | |
| (CMCoFx1)* | | |
| MCoFx2 | NH₂-DGGICPRILVYCRRDSDCPGACICIRRTYCGSGS-OH | 74 |
| acyclic | | |
| MCoFx2 | [DGGICPRILVYCRRDSDCPGACICIRRTYCGSGS] | 75 |
| cyclic | | |
| (cMCoFx2)* | | |
| MCoFx3 | NH₂-DGGRCPRLLRWCRRDSDCPGACICARGGLCGSGS-OH | 76 |
| acyclic | | |
| MCoFx3 | [DGGRCPRLLRWCRRDSDCPGACICARGGLCGSGS] | 77 |
| cyclic | | |
| (cMCoFx3)* | | |
| MCoFx4 | NH₂-DGGVCPRVGWRCRRDSDCPGACICYPTKWCGSGS-OH | 78 |
| acyclic | | |
| MCoFx4 | [DGGVCPRVGWRCRRDSDCPGACICYPTKWCGSGS] | 79 |
| cyclic | | |
| (cMCoFx4)* | | |
| MCoFx5 | NH₂-DGGRCCGGYLVCRRDSDCPGACICVFKKHCGSGS-OH | 80 |
| acyclic | | |
| MCoFx5 | [DGGRCCGGYLVCRRDSDCPGACICVFKKHCGSGS] | 81 |
| cyclic | | |
| (cMCoFx5)* | | |

*amide bond between first and last residues in sequence displayed above

TABLE 6
HIGH RESOLUTION MASS SPECTROMETRY DATA OF SYNTHETIC
PEPTIDES MEASURED BY MALDI-TOF MASS SPECTROSCOPY

| | Acyclic | | Cyclic | |
|---|---|---|---|---|
| Peptide | [M + H]ₑₓₚ | [M + H]_{obs} | [M + H]ₑₓₚ | [M + H]_{obs} |
| MCoTI-II | — | — | 3451.5008 | 3451.5083 |
| MCoFx1 | 3628.6023 | 3628.6676 | 3610.5917 | 3610.6080 |
| MCoFx2 | 3659.6220 | 3659.6309 | 3641.6115 | 3641.6121 |
| MCoFx3 | 3547.5557 | 3547.5793 | 3529.5451 | 3529.5670 |
| MCoFx4 | 3641.5173 | 3641.5363 | 3623.5070 | 3623.5012 |
| MCoFx5 | 3503.4416 | 3503.4586 | 3485.4310 | 3485.4676 |
| MCoTI-fxL1 | — | — | 3450.5123 | 3450.6022 |
| MCoTI-fxL5 | — | — | 3535.6630 | 3535.7626 |

Surface plasmon resonance (SPR) experiments demonstrated that all synthetic peptides exhibited FXIIa binding affinities in the nanomolar to picomolar range, regardless of backbone cyclization (refer to Table 7). cMCoFx1 exhibited the highest binding affinity, with a K_{D} of 900 μM, i.e. 60-fold higher affinity than MCoTI-II. Moreover, in vitro inhibition assays revealed that cMCoFx1 was the most active with a Ki of 370 μM, which is 350-fold more potent than MCoTI-II. In general, the backbone cyclic analogues of each molecule (except MCoFx5) displayed 2-7-fold higher binding affinity and inhibitory activity (i.e. lower K_{D} and Kᵢ values) than their acyclic forms. The only exception was cMCoFx5, which displayed a slight loss of activity compared with aMCoFx5.

TABLE 7
FXIIA BINDING AFFINITY (K_{D}) AND INHIBITORY
ACTIVITY (K_{I}) OF CHEMICALLY SYNTHESIZED
MCoTI-II AND ACYCLIC OR CYCLIC MCoFx1-5

| | K_{D} (nM) | | Kᵢ (nM) | |
|---|---|---|---|---|
| Peptide | Acyclic | Cyclic | Acyclic | Cyclic |
| MCoTI-II | | 58 ± 8 | | 129 ± 9 |
| MCoFx1 | 1.9 ± 0.04 | 0.90 ± 0.04 | 0.73 ± 0.2 | 0.37 ± 0.04 |
| MCoFx2 | 45 ± 3 | 10 ± 1 | 73 ± 3 | 12 ± 0.7 |
| MCoFx3 | 167 ± 40 | 23 ± 2 | 525 ± 30 | 75 ± 3 |
| MCoFx4 | 74 ± 6 | 14 ± 1 | 17 ± 0.9 | 5.5 ± 0.5 |
| MCoFx5 | 23 ± 0.4 | 82 ± 3 | 106 ± 7 | 207 ± 9 |

MCoTI-II was originally identified as a potent trypsin inhibitor and consistent with this, SPR measurements in our study showed a K_{D} of less than 100 μM for synthetic MCoTI-II towards bovine trypsin (refer to Table 8). Although FXIIa has 36% and 37% identity to human and bovine trypsin, respectively, the residues in the S1 pocket have far higher homology (˜90%). Thus, the selectivity of the MCoTI-II analogues was verified with respect to trypsin as an example of a related serine protease. K_{D} values of cMCoFx1-5 was determined, as well as aMCoFx1-5, to examine their selectivity by SPR. The most potent FXIIa inhibitor, cMCoFx1, exhibited 60-fold greater affinity for FXIIa and 300-fold lower affinity for trypsin compared with K_{D} values of MCoTI-II (refer to Table 8). Interestingly, cMCoFx2 showed improved binding to FXIIa (K_{D}=10 nM) compared with MCoTI-II (58 nM), yet its binding affinity also remained high to trypsin (K_{D} less than 100 μM). As cMCoFx1 and cMCoFx2 are identical at the P4, P2, P1 and P1′ sites, it is proposed that the selectivity of cMCoFx1 for FXIIa over trypsin must arise from differences between these two molecules at the P2′-P4′ sites and/or in loop 5. A similar trend was observed for cMCoFx3 and cMCoFx4, which exhibit 2-3-fold improved affinity for FXIIa compared to MCoTI-II, but nonetheless have 2-8-fold higher affinity for trypsin.

TABLE 8
FXIIA AND TRYPSIN BINDING AFFINITY OF
CHEMICALLY SYNTHESIZED CYCLIC MCoTI-II
AND ACYCLIC OR CYCLIC MCoFx1-5

| | K_{D} (nM) | | | |
|---|---|---|---|---|
| | FXIIa | | Trypsin | |
| Peptide | Acyclic | Cyclic | Acyclic | Cyclic |
| MCoTI-II | | 58 ± 7.5 | | <0.1 |
| MCoFx1 | 1.89 ± 0.04 | 0.90 ± 0.04 | 59.6 | 31.5 |
| MCoFx2 | 45.4 ± 2.9 | 10 ± 1.3 | <0.1 | <0.1 |
| MCoFx3 | 167 ± 42 | 22.8 ± 1.6 | 2.39 | 2.72 |
| MCoFx4 | 73.8 ± 6.3 | 14.4 ± 1.1 | 15.1 | 4.09 |
| MCoFx5 | 23.0 ± 0.4 | 81.8 ± 3.2 | No binding | No binding |

Neither aMCoFx5 nor cMCoFx5 showed detectable affinity for trypsin (refer to Table 8). Although the origin of this high selectivity is not clear, it is proposed that the unique C6 mutation at P2 site as well as consecutive G7 and G8 mutations at P1 and P1′ sites may alter the entire loop, resulting in presentation of a different loop structure or possibly altered folding compared with MCoTI-II. Such an unexpected structural change could eliminate the original interaction with trypsin occurring via loop 1, and instead create a new interface between MCoFx5 and FXIIa, leading to the observed highly selective interaction even though the inhibitory activity is relatively modest (Kᵢ=106 nM, refer to Table 7).

In addition to trypsin, the binding affinity of the selected peptides towards factor XII (FXII), the zymogen form of FXIIa was examined. SPR measurements revealed that none of the selected peptides exhibited a FXII-binding response at a concentration of 5 μM. The X-ray crystal structure of the FXII protease domain reveals that several key binding pockets are not properly formed in the zymogen, including the S1 pocket and oxyanion hole, which may explain the weak binding of active site targeted peptides, such as MCoTI-II analogues.

The cytotoxicity of cyclic MCoFx1 was assessed using human umbilical vein endothelial cells (HUVECs). Cyclic MCoFx1 was found to be not toxic to HUVECs (refer to FIG. 6).

### Example 3—Selectivity of cMCoFx1 Variants for FXIIa and the Intrinsic Coagulation Pathway

To further characterize the inhibitory activity of cMCoFx1, two chimeric peptides, referred to as cMCoTI-fxL1 and cMCoTI-fxL5 (refer to Table 9), were synthesized where the peptide motif from either loop 1 or loop 5 of cMCoFx1 was grafted into the respective loop of MCoTI-II (refer to Table 6 for MS data). A comparison of the α-proton NMR chemical shifts of cMCoTI-fxL1 and cMCoTI-fxL5 (refer to FIG. 5) showed they were similar to their parent peptides in their respective regions. For instance, the secondary Ha shifts for loop 1 of cMCoTI-fxL1 were similar to those of cMCoFx1, whereas loop 1 of cMCoTI-fxL5 was comparable to MCoTI-II.

TABLE 9
SEQUENCES OF CHIMERIC PEPTIDES

| | | SEQ |
|---|---|---|
| | | ID |
| Peptide | Sequence | NO |
| CMCoTI- | [DGGICPRIGRLCRRDSDCPGACICRGNGYCGSGS] | 82 |
| fxL1* | | |
| CMCoTI- | [DGGVCPKILKKCRRDSDCPGACICRATRFCGSGS] | 83 |
| fxL5* | | |

*amide bond between first and last residues in sequence displayed above

The activity screen for cMCoFx1 and the single loop-grafted variants was then expanded from FXIIa to other clinically relevant serine proteases (refer to Table 10). These assays revealed that the difference in Kᵢ for cMCoFx1 against FXIIa (0.37 nM) and trypsin (1,110 nM) exceeds three orders of magnitude. Remarkably, cMCoFx1 shows almost no inhibitory activity against other serine proteases, confirming that cMCoFx1 is highly specific for FXIIa. cMCoTI-fxL1 exhibits a similar profile to cMCoFx1, although its potency against FXIIa (Kᵢ=0.69 nM) and selectivity with respect to trypsin and matriptase (Kᵢ values of 996 and 2,210 nM, respectively) are slightly lower. By contrast, cMCoTI-fxL5 displays nearly the same potency and selectivity as MCoTI-II, i.e. Kᵢ values for FXIIa, trypsin and matriptase are 66, 0.08, and 17 nM, respectively. These results clearly demonstrate that the sequence of loop 1 plays a major role in determining the potency and selectivity of MCoTI-II analogues, although the sequence of loop 5 does provide a subtle enhancement to the overall activity of cMCoFx1.

TABLE 10
INHIBITORY ACTIVITY OF cMCoFx1, cMCoTI-FxL1 AND
cMCoTI-FxL5 AGAINST A PANEL OF SERINE PROTEASES

| | cMCoFx1 | cMCoTI-fxL1 | cMCoTI-fxL5 |
|---|---|---|---|
| FXIIa | Kᵢ = 0.37 ± 0.04 nM | Kᵢ = 0.69 ± 0.04 nM | Kᵢ = 66 ± 0.8 nM |
| Trypsin | Kᵢ = 1110 ± 36 nM | Kᵢ = 996 ± 17 nM | Kᵢ = 0.08 ± 0.005 nM |
| FXa | IC₅₀ > 5 μM (0%) | IC₅₀ > 5 μM (0%) | IC₅₀ > 5 μM (0%) |
| FXIa | IC₅₀ > 5 μM (0%) | IC₅₀ > 5 μM (1%) | IC₅₀ > 5 μM (1%) |
| Thrombin | IC₅₀ > 5 μM (2%) | IC₅₀ > 5 μM (1%) | IC₅₀ > 5 μM (3%) |
| Plasma kallikrein | IC₅₀ > 5 μM (0%) | IC₅₀ > 5 μM (0%) | IC₅₀ > 5 μM (0%) |
| Plasmin | IC₅₀ > 5 μM (13%) | IC₅₀ > 5 μM (19%) | Kᵢ = 31 ± 4 nM |
| uPA | IC₅₀ > 5 μM (0%) | IC₅₀ > 5 μM (0%) | IC₅₀ > 5 μM (0%) |
| tPA | IC₅₀ > 5 μM (0%) | IC₅₀ > 5 μM (1%) | IC₅₀ > 5 μM (1%) |
| Matriptase | Kᵢ = 9120 ± 760 nM | Kᵢ = 2210 ± 130 nM | Kᵢ = 17 ± 0.8 nM |

Kᵢ was determined for inhibitors with IC₅₀ < 5 μM against a given protease. The percentage value in brackets indicates percent inhibition at 5 μM for the off-target proteases.

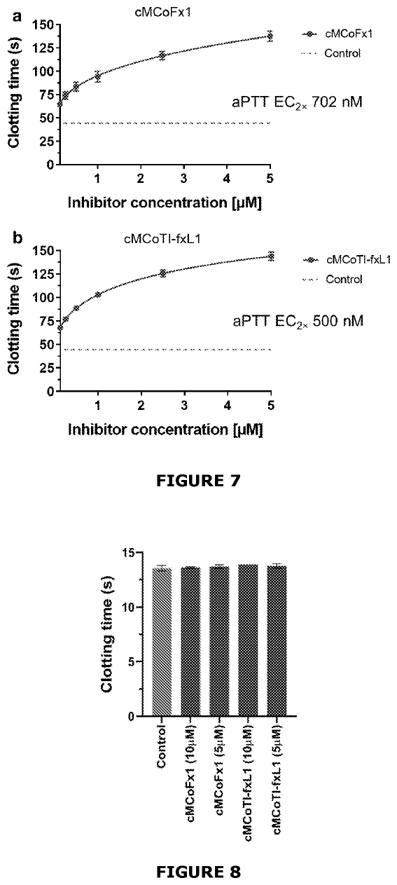

Having identified two potent and selective FXIIa inhibitors (cMCoFx1 and cMCoTI-fxL1), coagulation assays were performed to assess their biological activity in human plasma. Inhibition of FXIIa was examined in activated partial thromboplastin time (aPTT) assays, where addition of kaolin initiates the intrinsic pathway via activation of FXII. Both inhibitors prolonged the clotting time in a dose-dependent manner and maintained substantial activity in the nanomolar range (refer to FIG. 7), as seen by the concentration of inhibitor required to double the clotting time observed in control assays (EC_{2×}). By this measure, cMCoTI-fxL1 was slightly more potent than cMCoFx1 (EC_{2×}=500 nM vs 702 nM), although this subtle difference between the two inhibitors could be due to variation in the experimental conditions between coagulation assays and prior enzyme kinetic assays.

Prothrombin time (PT) assays were also performed to confirm that both inhibitors are selective for the intrinsic pathway (refer to FIG. 8). No significant differences in clotting time compared to control assays were observed for cMCoFx1 and cMCoTI-fxL1 at 10 μM, demonstrating that the potent and selective activity of these inhibitors identified in biochemical assays extends to biological assays in human plasma.

### Example 4—Stability of cMCoFx1 in Human Serum

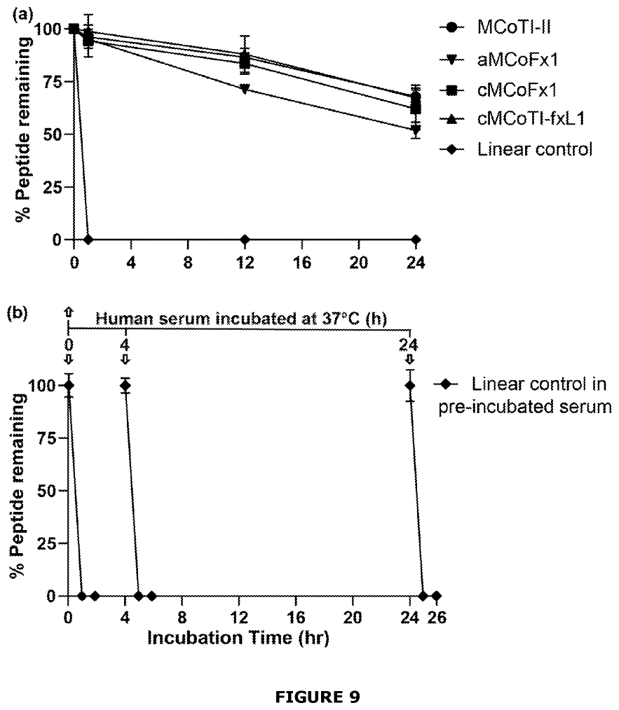

The stability of aMCoFx1, cMCoFx1, and cMCoTI-fxL1 compared to MCoTI-II was evaluated in human serum for up to 24 h at 37° C. (refer to FIG. 9). All tested peptides showed high serum stability with half-lives of more than 24 hours. A linear control peptide (EAIYAAPFAKKK) rapidly degraded in serum within 1 h. The results clearly showed that aMCoFx1, cMCoFx1, and cMCoTI-fxL1 are highly resistant to serum proteases similar to MCoTI-II.

### Example 5—Activity of Acyclic MCoFx1 Variant

An acyclic variant of MCoFx1 was designed based on a minimized knottin scaffold (29 amino acids) and synthesized using solid phase peptide synthesis (NH₂-RICPRIGRLCRRDSDCPGACICRATRFCG-OH [SEQ ID NO: 84]). The variant includes the modified sequences in loop 1 and loop 5 identified by mRNA display (refer to Examples 1 and 2), and was found to be a potent FXIIa inhibitor (Kᵢ=1.4 nM). Variants based on an acyclic knottin scaffold may simplify large scale chemical synthesis or recombinant expression.

### Example 6—Synthesis of MCoFx1 Variants with a Lysine Residue

Three MCoFx1 variants that have a lysine residue in loop 2 or loop 6 were designed (refer to Table 11). For these variants, Tyr33 was mutated back to serine as per the original mRNA display screen (FIG. 1), with the exception of the [S33K]MCoFx1 mutant, where this residue was mutated to a lysine. Cyclic [S33K]MCoFx1 was synthesised using solid phase peptide synthesis and was successfully used for attachment of a fluorescent label (NHS-Alexa488), indicating that this site may be used for attachment of a protecting or stabilizing moiety, such as a lipid moiety.

TABLE 11
SEQUENCES OF MCoFx1 VARIANTS

| | | SEQ |
|---|---|---|
| | | ID |
| Peptide | Sequence | NO |
| Cyclic | [GGICPRIGRLCKRDSDCPGACICRATRFCGSGSD] | 85 |
| [R12K] | | |
| MCoFx1* | | |
| Cyclic | [GGICPRIGRLCRKDSDCPGACICRATRFCGSGSD] | 86 |
| [R13K] | | |
| MCoFx1* | | |
| (also | | |
| referred | | |
| to herein | | |
| as [R13K] | | |
| MCoFx7) | | |
| Cyclic | [GGICPRIGRLCRRDSDCPGACICRATRFCGSGKD] | 87 |
| [S33K] | | |
| MCoFx1* | | |

*amide bond between Gly1 and Asp34

### Example 7—Determining FXIIa Specificity in Loop 1 Using Synthetic MCoTI-II Libraries

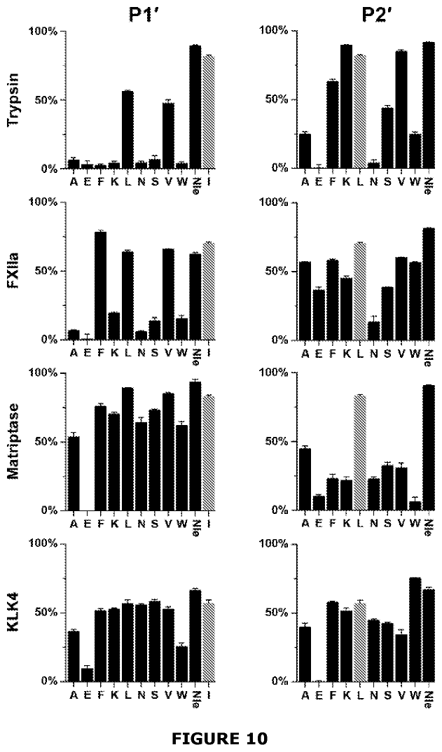

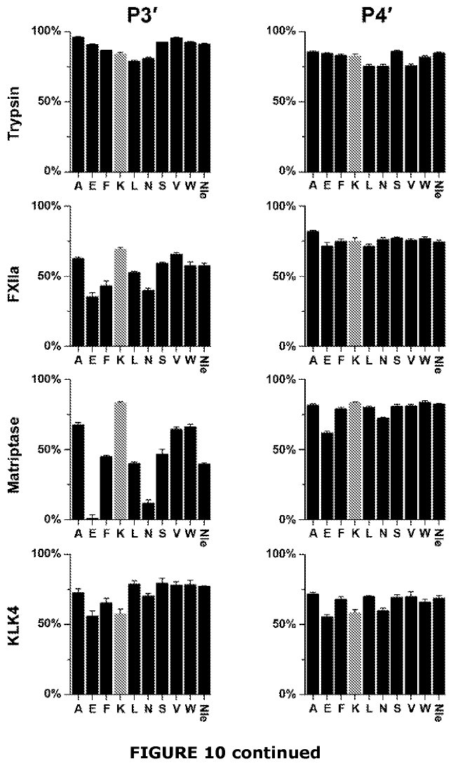

In parallel with the mRNA display affinity selection, synthetic peptide libraries were generated to characterize the specificity of FXIIa at four positions in loop 1 (P1′ to P4′) of MCoTI-II (refer to FIG. 1C). The libraries were based on an acyclic MCoTI-II variant (M, refer to Table 12) and comprised ten variants that were individually synthesized with one of ten amino acids (Ala, Glu, Phe, Lys, Leu, Asn, Ser, Val, Trp or Nle) substituted in the target position (10 peptides×4 positions) (refer to FIG. 10). These amino acids largely cover the chemical diversity evident among proteinogenic amino acids.

The inhibitor variants were screened against FXIIa and three off-target proteases, trypsin, matriptase and kallikrein-related peptidase 4 (KLK4), in competitive inhibition assays using a single concentration of inhibitor that ranged from 1.25 nM (KLK4) to 25 nM (FXIIa). Activity data is provided in FIG. 10.

Specificity data for the four proteases revealed that, although each residue at P1′-P4′ (corresponding to residues 6-9 of M; refer to Table 12) in MCoTI-II was broadly favored, all positions were amenable to substitution. Residues in close proximity to the scissile bond (P1′ and P2′) had particularly strong influence on inhibitory activity and selectivity. At the P1′ position, Ile is highly conserved in knottin protease inhibitors and hydrophobic residues were preferred by trypsin, led by Ne and Ile. However, a broader range of amino acids was favored by other enzymes that included Phe (FXIIa) and hydrophobic or polar residues (matriptase and KLK4). At the P2′ position, the most common residue in nature-derived knottin protease inhibitors is Leu, which was well-tolerated by all enzymes screened. Trypsin, FXIIa, and KLK4 favored several additional amino acids, including Lys (trypsin and KLK4) or aromatic residues (FXIIa and KLK4). Additionally, FXIIa appeared to be the only enzyme that tolerated Glu at the P2′ position. By contrast, the P2′ specificity of matriptase appeared to be relatively narrow, with only Ne or Leu generating potent inhibitors.

Amino acid substitutions at P3′ and P4′ had less impact on inhibitory activity and selectivity. For trypsin and KLK4, each substitution at P3′ produced little change in activity compared to Lys (present in MCoTI-II). Similarly, most P3′ residues were well-tolerated by FXIIa, although variants with Phe, Asn, and Glu showed less potent activity. P3′ substitutions had larger effects on matriptase inhibition, with Lys, Ala, Trp, and Val being highly preferred, whereas Asn and Glu produced variants with weak activity. Substituting the P4′ residue did not lead to marked changes in activity against any of the proteases screened. Matriptase and KLK4 showed some degree of amino acid specificity, with Glu and Asn slightly less preferred than other amino acids.

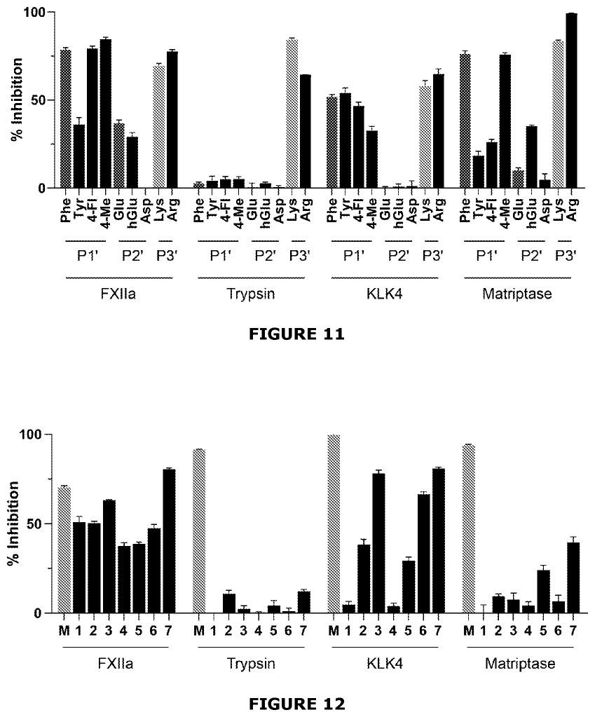

Differences in the P1′-P2′ specificity of FXIIa compared to the other enzymes revealed several substitutions of interest. At P1′, Phe was the optimal residue for FXIIa but poorly favored by trypsin, whereas at P2′, Glu was tolerated by FXIIa, but not by any of the other enzymes screened. To explore the chemical space around these hits, additional variants were synthesized with related proteinogenic or non-proteinogenic amino acids. For P1′ Phe, modifications at the para position of the phenyl ring (Tyr, 4-fluoro-L-Phe, and 4-methyl-L-Phe) were tested. Both non-proteinogenic amino acids were slightly more preferred by FXIIa compared to Phe, but Tyr was poorly favored (refer to FIG. 11). Each variant maintained weak activity against trypsin, whereas 4-methyl-L-Phe provided greater selectivity over KLK4 and 4-fluoro-L-Phe gave better selectivity over matriptase (refer to FIG. 11). For P2′ Glu, the sidechain length was varied using Asp or homoGlu (hGlu). Asp was poorly favored by all four enzymes, and hGlu led to improved activity against matriptase but not FXIIa (refer to FIG. 11). P3′ Lys and Arg were also compared to select the optimal basic residue. P3′ Arg led to slightly improved activity against FXIIa, but also for matriptase and KLK4 (refer to FIG. 11).

With the additional specificity data in hand, a second peptide library was synthesized to test various sequence combinations for FXIIa. The P1′ residue was fixed as 4-fluoro-L-Phe, which was highly preferred by FXIIa but not trypsin or matriptase. At P2′, residues were selected that were preferred by FXIIa but not favored by one or more off-targets (Trp and Val), as well as Glu as it was poorly favored by all off-targets. Additionally, both P3′ residues (Lys and Arg) were included to account for any cooperativity effects with 4-fluoro-L-Phe at P1′. Inhibitor variants containing all possible combinations of these amino acids were produced by synthesizing six peptides (1-6, refer to Table 12). For comparison, we also included the wild-type analogue (M) and a variant with the most-preferred P1′-P4′ residues for FXIIa (7): 4-fluoro-L-Phe, Nle, Lys, Ala. The most potent FXIIa inhibitor was 7, which showed a slight improvement in activity compared to the wild-type knottin (M) but limited selectivity over KLK4 and matriptase (refer to FIG. 12). Variants with P2′ Glu (1 and 4) showed the highest overall selectivity compared to inhibitors with P2′ Val (2 and 5) or Trp (3 and 6). Additionally, each inhibitor with P3′ Lys (1-3) outperformed the corresponding variant with P3′ Arg (4-6) for FXIIa, trypsin, and KLK4, but not matriptase.

TABLE 12
SEQUENCES OF MCoTI-II VARIANTS

| | | SEQ ID |
|---|---|---|
| Peptide | Sequence | NO |
| M | NH₂-RVCPRILKKCRRDSDCPGACICRGNGYCG-OH | 88 |
| 1 | NH₂-RVCPR[4-F-Phe]EKKCRRDSDCPGACICRGNGYCG-OH | 89 |
| 2 | NH₂-RVCPR[4-F-Phe]VKKCRRDSDCPGACICRGNGYCG-OH | 90 |
| 3 | NH₂-RVCPR[4-F-Phe]WKKCRRDSDCPGACICRGNGYCG-OH | 91 |
| 4 | NH₂-RVCPR[4-F-Phe]ERKCRRDSDCPGACICRGNGYCG-OH | 92 |
| 5 | NH₂-RVCPR[4-F-Phe]VRKCRRDSDCPGACICRGNGYCG-OH | 93 |
| 6 | NH₂-RVCPR[4-F-Phe]WRKCRRDSDCPGACICRGNGYCG-OH | 94 |
| 7 | NH₂-RVCPR[4-F-Phe][Nle]KACRRDSDCPGACICRGNGYCG-OH | 95 |
| Cyclic* | [GGVCPR[4-F-Phe]EKKCRRDSDCPGACICRGNGYCGSGSD] | 96 |

*amide bond between Gly1 and Asp34

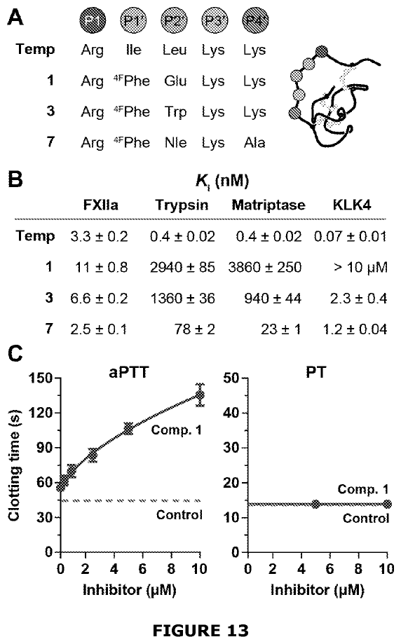

The activity of peptides 1, 3 and 7 was characterized by determining Kᵢ values for each enzyme (refer to FIG. 13). Compared to M (template, also referred to as “temp” herein), the non-selective variant 7 showed 1.3-fold improved activity against FXIIa and 17- to 195-fold weaker activity against the off-targets (refer to FIG. 13B). Replacing P2′ Nle with Trp maintained activity against FXIIa and KLK4, but decreased inhibition of trypsin and matriptase by 17- to 40-fold (refer to FIG. 13B). The most selective variant (1) also showed potent activity against FXIIa, but the Kᵢ value against trypsin or matriptase was in the micromolar range, and the inhibitor displayed even weaker activity against KLK4 (refer to FIG. 13B). For FXIIa, this level of activity represents only a three-fold change in Kᵢ compared to M, even though the inhibitor has P2′ Glu which is not optimal for potency. However, changes in activity for off-target enzymes were 7,350-fold for trypsin, 9,650-fold for matriptase, and at least an additional order of magnitude (>100,000-fold) for KLK4. Selectivity analyses against other human serine proteases (thrombin, FXa, FXIa, plasma kallikrein, plasmin, uPA, tPA) further verified the high selectivity of 1. Strikingly, the potency and selectivity of M (Temp) may be increased with two amino acid substitutions: P1′ Ile to 4-fluoro-L-Phe, and P2′ Leu to Glu.

Coagulation assays in human plasma were performed to assess the biological activity of the selective FXIIa inhibitor (1). Activation of the coagulation system is mediated by two converging protease cascades: the intrinsic/contact pathway and the extrinsic/tissue factor pathway. FXIIa is the lead enzyme in the intrinsic pathway, and its activation can be measured in activated partial thromboplastin time assays. Inhibitory activity is observed as a delay in clotting time compared to control plasma without inhibitor. For 1, a dose-response effect on plasma clotting was observed (refer to FIG. 13C), with the inhibitor concentration required to extend the clotting time by 50% (EC_{1.5×}) calculated as 840 nM, whereas doubling the clotting time (EC_{2×}) required 2.9 μM inhibitor. To verify the selectivity of 1 for FXIIa and the intrinsic pathway, prothrombin time assays were performed to measure clotting via the extrinsic pathway (refer to FIG. 13C). No effect on clotting time was observed at 10 μM inhibitor.

### Example 8—Synthesis and Activity of Cyclic MCoFx6 ([I7]IMCoFx1)

Cyclic [I7F]MCoFx1 (also referred to as cyclic MCoFx6) was synthesized using solid phase peptide synthesis ([GGICPRFGRLCRRDSDCPGACICRATRFCGSGSD] [SEQ ID NO: 97]). For this inhibitor, Tyr33 was mutated back to serine as per the original mRNA display screen (FIG. 1). Cyclic MCoFx6 was screened using a competitive inhibition assay, with the substrate being changed from a colorimetric substrate (Ac-QRFR-pNA in the data of Tables 7 and 10) to a fluorescent substrate (Boc-QGR-MCA) to allow the assay to be run with a lower concentration of enzyme. The assay was performed as described for Examples 2 and 3, except that the FXIIa concentration was 0.5 nM and the substrate concentration was 50 μM (K_{M}=187 μM). Cyclic MCoFx6 was found to have a Kᵢ of 1090±59 μM.

### Example 9—Saturation Mutagenesis of MCoFx1

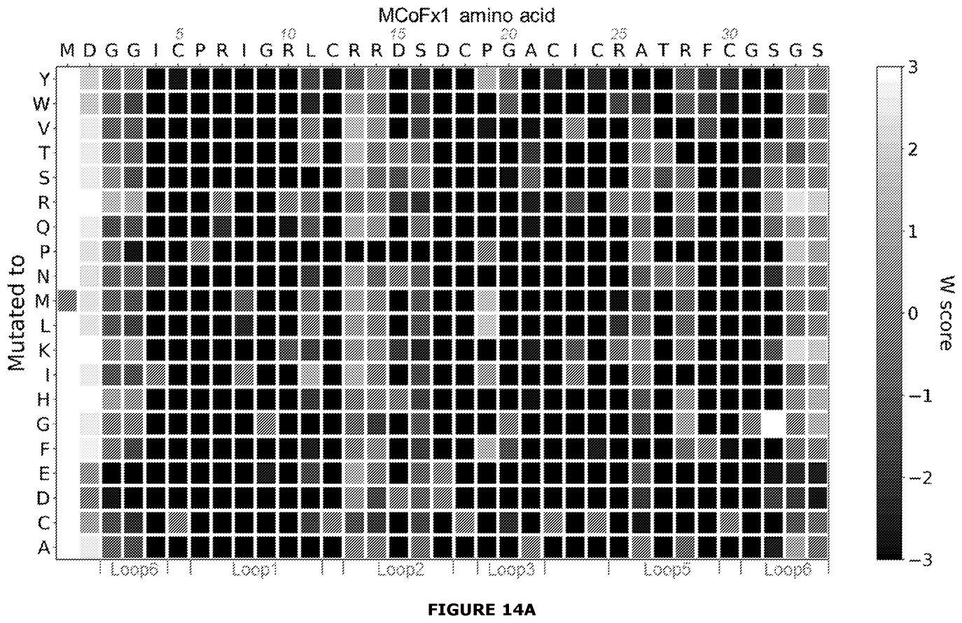

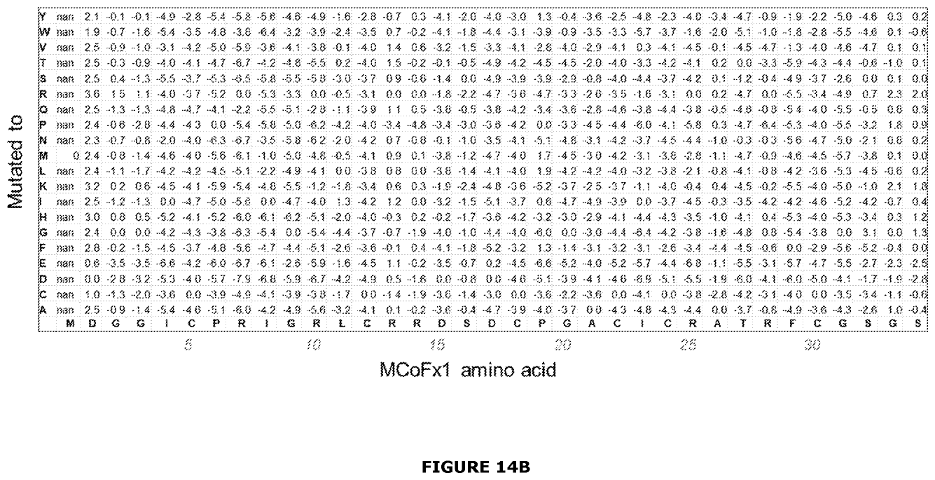

The effect of modifications outside loops 1 and 5 of MCoFx1 was investigated by performing a saturation mutagenesis scanning of the selected MCoFx1 sequence. The mutants library was designed based on the mRNA display selected sequence of MCoFx1 (MDGGICPRIGRLCRRDSDCPGACICRATRFCGSGSGS [SEQ ID NO: 98]), with a random residue (containing the possibility of all 20 naturally occurring amino acids) replacing the parental residue at the cyclotide core positions (DGGICPRIGRLCRRDSDCPGACICRATRFCGSGS [SEQ ID NO: 99]) in each mutant sequence. mRNA templates of the parent and mutants were mixed equally, puromycin-ligated, in vitro translated, reverse transcribed, and purified with HA-tag purification, followed by a streptavidin-based FXIIa pulldown, after which the library was separated into “binding” and “non-binding” fractions. cDNAs from each fraction were sequenced using next-generation sequencing (NGS). As described previously in Vinogradov et al. (2020), J Am Chem Soc, 142: 20329-20334, data was utilized from both the “binding” and “non-binding” fractions to increase the signal response and overall accuracy of the method. For every library mutant, the Y-score was defined as the ratio of peptide's frequencies in “binding” and “non-binding” populations and W-score as subtraction of the log 2Y of parent MCoFx1 from every mutant. W-scores of the single-position mutants are presented in FIGS. 14A and 14B. Reporting of the W-scores makes for a uniform perception of the results, with higher scores corresponding to binding-beneficial mutations.

As indicated by the W-scores (FIGS. 14A and 14B), the mutations in loop 1 mainly decreased the binding affinity to FXIIa, whereas mutations at A26 to T/S/P/K/R and R28 to H/G in loop 5 slightly improved the mutant's target-binding affinity. Interestingly, several beneficial mutations in loops 2, 3, and 6 were observed, including R13, R14 and P19 to hydrophobic residues (I/L) or aromatic residues (F/Y/W), D1 to almost all other residues, and G2, G3, G33 and S34 to K/R.

### Example 10—Activity of Cyclic MCoFx7 ([Y33S]MCoFx1) VARIANTS

Additional cyclic variants were designed on the basis of the results of Example 9 (refer to Table 13). For this inhibitor series, Tyr33 was mutated back to serine as per the original mRNA display screen (FIG. 1). These peptides were synthesised using solid phase peptide synthesis.

TABLE 13
SEQUENCES OF CYCLIC MCoFx7 VARIANTS

| Peptide | Sequence | SEQ ID NO |
|---|---|---|
| Cyclic MCoFx7 | [GGICPRIGRLCRRDSDCPGACICRATRFCGSGSD] | 157 |
| ([Y33S]MCoFx1)* | | |
| Cyclic | [GGICPRIGRLCRRDSDCPGACICRPTRFCGSGSD] | 158 |
| [A25P]MCoFx7* | | |
| Cyclic | [GGICPRIGRLCRRDSDCPGACICRKTRFCGSGSD] | 159 |
| [A25K]MCoFx7* | | |
| Cyclic | [GGICPRIGRLCRRDSDCPGACICRATGFCGSGSD] | 160 |
| [R27G]MCoFx7* | | |
| Cyclic | [GGICPRIGRLCRRDSDCPGACICRATHFCGSGSD] | 161 |
| [R27H]MCoFx7* | | |
| Cyclic | [GGICPRIGRLCVRDSDCPGACICRATRFCGSGSD] | 162 |
| [R12V]MCoFx7* | | |
| Cyclic | [GGICPRIGRLCERDSDCPGACICRATRFCGSGSD] | 163 |
| [R12E]MCoFx7* | | |
| Cyclic | [GGICPRIGRLCTRDSDCPGACICRATRFCGSGSD] | 164 |
| [R12T]MCoFx7* | | |
| Cyclic | [GGICPRIGRLCRKDSDCPGACICRATRFCGSGSD] | 86 |
| [R13K]MCoFx7* | | |
| (also referred to | | |
| herein as | | |
| [R13K]MCoFx1) | | |
| Cyclic | [GGICPRIGRLCRRDSDCPGACICRATRFCGSGSP] | 165 |
| [D34P]MCoFx7* | | |

*amide bond between first and last residues in sequences displayed above.

The new variants were screened using competitive inhibition assays, with the substrate being changed from a colorimetric substrate (Ac-QRFR-pNA in the data of Tables 7 and 10) to a fluorescent substrate (Boc-QGR-MCA) to allow the assay to be run with a lower concentration of enzyme. Assays were performed as described for Examples 2 and 3, except that the FXIIa concentration was 0.5 nM and the substrate concentration was 50 μM (K_{M}=187 μM). Using this more sensitive assay, the Kᵢ of cyclic MCoFx7 was found to be 64 μM (refer to Table 14). The effect of amino acid substitutions within loop 2 (Arg12, Arg13), loop 5 (Ala25, Arg27) or loop 6 (Asp34) on the activity of cyclic MCoFx7 against FXIIa was determined (refer to Table 14). The mutagenesis screen indicated that several amino acids might be well-tolerated in place of Arg12, and variants where this residue was mutated to Val or Thr showed near-identical activity to cyclic MCoFx7. Additionally, mutating Arg to Glu (reversing the side chain charge) only led to two-fold weaker activity. The adjacent residue Arg13 could also be mutated to Lys with no change in activity. Within loop 5, mutating Ala25 to Lys produced a slight gain in activity, whereas the Ala to Pro mutation led to a slight decrease in activity. Both mutations at Arg27 led to losses in activity in the order of two-fold (His) or 3.2-fold (Gly). The Asp34 to Pro mutation in loop 6 also maintained potent activity (Kᵢ=91 μM).

TABLE 14
FXIIA INHIBITORY ACTIVITY OF
CYCLIC MCoFx7 VARIANTS

| Peptide Variant | Kᵢ (pM) |
|---|---|
| Cyclic MCoFx7 | 64 ± 4 |
| Cyclic [R12V]MCoFx7 | 69 ± 5 |
| Cyclic [R12T]MCoFx7 | 65 ± 5 |
| Cyclic [R12E]MCoFx7 | 130 ± 7 |
| Cyclic [R13K]MCoFx7 | 68 ± 3 |
| Cyclic [A25P]MCoFx7 | 93 ± 5 |
| Cyclic [A25K]MCoFx7 | 46 ± 5 |
| Cyclic [R27G]MCoFx7 | 210 ± 8 |
| Cyclic [R27H]MCoFx7 | 120 ± 8 |
| Cyclic [D34P]MCoFx7 | 91 ± 6 |

### Example 11—Anticoagulant Activity in Human Whole Blood

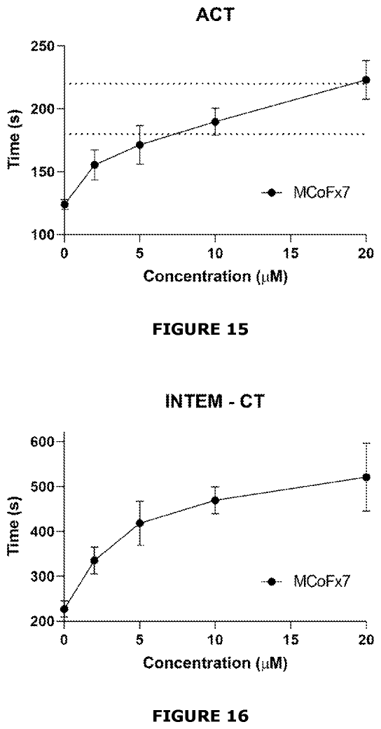

The anticoagulant activity of cyclic MCoFx7 was determined in human whole blood using two assays: activated clotting time (ACT) and rotational thromboelastometry (ROTEM). In ACT assays, the clotting time (y-axis) was increased in a dose-dependent manner from the control (124 s), reaching 223 s with 20 μM MCoFx7. 10 μM or 20 μM cyclic MCoFx7 (refer to Table 13) produced a clotting time that is within the therapeutic range (180-220 s, indicated by the dotted lines) for patients on ECMO receiving the standard-of-care anticoagulant (heparin) (refer to FIG. 15). A single concentration of cyclic [A25P]MCoFx7 (refer to Table 13) was also tested (10 μM) and this peptide inhibitor showed similar activity (clotting time=186 s). Anticoagulant activity was verified by thromboelastometry (TEM) that was measured after activation of the intrinsic pathway (INTEM) (refer to FIG. 16). For cyclic MCoFx7, the clotting time was also increased in a dose-dependent manner from the control (227 s), reaching 521 s with 20 μM peptide inhibitor. By comparison, a single concentration of cyclic [A25P]MCoFx7 (10 μM) produced a clotting time of 500 s, similar to cyclic MCoFx7 at 10 μM (469 s).

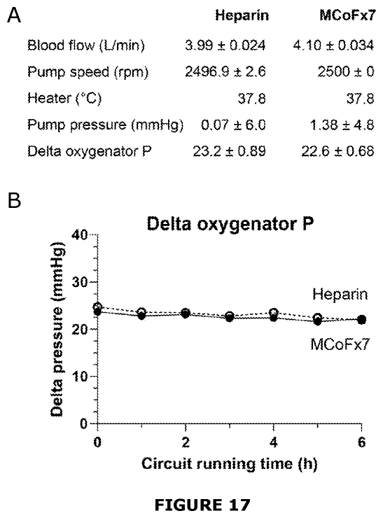

Cyclic MCoFx7 was subsequently tested as a replacement for the standard-of-care heparin in an ex vivo extracorporeal membrane oxygenation (ECMO) model. This experiment used a circuit setup based on the Permanent Life Support (PLS) System (Maquet C P, Rastatt, Germany) consisting of a Quadrox D Oxygenator and ROTAFLOW centrifugal pump that were incorporated into a tubing set with a tip-to-tip BIOLINE (albumin and heparin) coating. Human blood (470 mL) treated with heparin (initial dose: 350 UI, then 10 UI at 1 h, 20 UI at 2 h, 10 UI at 3 h, and 10 UI at 4 h) or cyclic MCoFx7 (single dose: 20 μM) was circulated in the ECMO system for 6 h, and blood samples were taken at 30 min, 2 h, 4 h, and 6 h for analysis. Circuit parameters were also monitored, indicating that cyclic MCoFx7 maintained similar blood flow rate, pump speed and pump pressure to heparin (refer to FIG. 17). Additionally, the difference in pressure at the inlet and outlet of the membrane oxygenator was monitored (reported as delta oxygenator pressure [P]), which allows for detection of clots that might lodge in the oxygenator. The delta oxygenator P was stable over the 6 h time course for both cyclic MCoFx7 and heparin (˜23 mmHg; refer to FIG. 17).

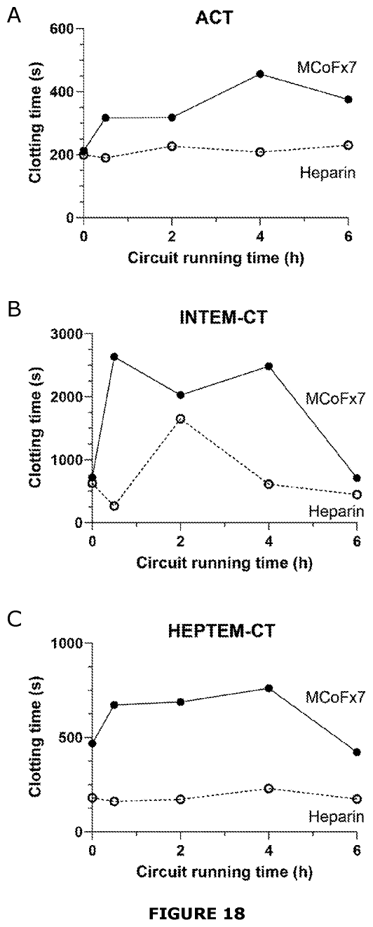

The duration of effect for each treatment was also monitored by taking blood samples from the circuit over time and performing clotting assays (refer to FIG. 18). In ACT assays, a clotting time of more than 200 s was maintained for cyclic MCoFx7 over the course of the experiment (>300 s from 0.5 h-6 h). Similarly, INTEM assays indicated that anticoagulant activity was maintained for 6 h. HEPTEM assays (similar to INTEM assays except that heparinase is added to degrade heparin present in the blood sample) verified that the anticoagulant activity for cyclic MCoFx7 was independent of heparin.

### Materials and Methods

All chemical reagents were purchased from Watanabe Chemical Industry, Nacalai Tasque, Tokyo Chemical Industry, Sigma-Aldrich Japan or Wako. Unless otherwise noted, all the chemical reagents obtained from commercial sources were used without any purification. H₂O used for buffer preparations was from a Sartorius filtration system (18.2M).

### MCoTI-II-Based Library Generation

Oligonucleotides corresponding to the designed library were ordered from Eurofins Genomics, with the 12 random residues in loop 1, 5 and 6 encoded with NNK codon (N=A, T, G, C and K=G, T) (refer to Table 4). The DNA library was constructed in a two-step PCR reaction using Q5 high-fidelity DNA polymerase (New England Biolabs), with the first extension step extending two pieces of oligos, MCoTI-II-NNK7.F80 and MCoTI-II-NNK5.R82, containing the whole peptide-coding region, while the second amplification step added upstream T7 promoter, GGG triplet, epsilon sequence and ribosome binding (Shine-Dalgarno) sequence and downstream puromycin linker binding sequence. The PCR reaction was conducted in 1×Q5 reaction buffer (New England Biolabs), 200 μM each dNTPs, 0.5 μM forward and reverse primers, 1% (v/v) 1×Q5 High-Fidelity DNA Polymerase (New England Biolabs), and sequential PCR conditions were listed in Table 15. To reach the library diversity of 10¹⁴ molecules, the first-step PCR was conducted in 650 μL scale and added directly to the second-step PCR (6500 μL scale), together with the other PCR recipes. The PCR products were extracted by phenol/chloroform, precipitated by ethanol, dissolved in 650 μL water, and used for in vitro transcription at 37° C. for 16 h in a 6500 μL reaction scale. The in vitro transcription reaction mixture contained 40 mM Tris-HCl, 1 mM spermidine, 0.01% (v/v) Triton X-100, 10 mM DTT, 30 mM MgCl₂, 5 mM NTPs, 30 mM KOH, 650 μL template DNA solution, 0.12 μM home-made T7 RNA polymerase at pH 8.0. The resulting mRNA transcripts were precipitated by adding 10% (v/v) 3M NaCl and 80% (v/v) of isopropanol followed by centrifuge. After wash with 70% (v/v) EtOH, the pellet was dissolved in water with 10% (v/v) volume of transcription reaction and equivalent volume of 2× RNA loading buffer (8M urea, 2 mM Na₂EDTA·2H₂O, 2 mM Tris-HCl, pH 7.5) was added. After heating at 95° C. for 2 mins, the mRNA was purified by 8% (v/v) polyacrylamide gel containing 6 M urea. The correct band was visualized by UV light, cut out and extracted in 0.3 M NaCl solution for at least 5 hours. After removal of the gel by centrifuge and filtration, twice volume of ethanol was added and the mRNA was precipitated by centrifuge at 13,000 rpm for 15 mins. The pellet was washed with 70% (v/v) ethanol, dried at room temperature and dissolved in H₂O to 10 PM.

TABLE 15
PCR REACTION CONDITIONS
FOR LIBRARY ASSEMBLY
Extension PCR for library construction

| | | | | |
|---|---|---|---|---|
| 98° C. | 1 | min | 1 | cycle |
| 66° C. | 1 | min | 5 | cycles |
| 72° C. | 1 | min | | |

Amplification PCR for library construction

| | | | | |
|---|---|---|---|---|
| 98° C. | 40 | s | 6 | cycles |
| 52° C. | 40 | s | | |
| 72° C. | 40 | s | | |

mRNA Display Against FXIIa

The mRNA template of MCoTI-II-based library was covalently linked to a puromycin linker (refer to Table 4) using home-made T4 RNA ligase, before in vitro translated using a translation cocktail as previously described in Goto et al. (2011) Nat Protoc, 6: 779-790. In brief, the translation mixture consisted of 50 mM HEPES-KOH (pH 7.6), 100 mM potassium acetate, 12.3 mM magnesium acetate, 2 mM ATP, 2 mM GTP, 1 mM CTP, 1 mM UTP, 20 mM creatine phosphate, 2 mM spermidine, 1 mM dithiothreitol, 100 μM 10-formyl-5,6,7,8-tetrahydrofolic acid, 1.5 mg ml⁻¹ E. coli total tRNA, 1.2 μM E. coli ribosome, 0.6 μM methionyl-tRNA formyltransferase, 2.7 μM IF1, 0.4 μM IF2, 1.5 μM IF3, 0.26 μM EF-G, 10 μM EF-Tu/EF-Ts complex, 0.25 μM RF2, 0.17 μM RF3, 0.5 μM RRF, 4 μg ml⁻¹ creatine kinase, 3 μg ml⁻¹ myokinase, 0.1 μM inorganic pyrophosphatase, 0.1 μM nucleotide diphosphate kinase, 0.1 μM T7 RNA polymerase, 0.73 μM AlaRS, 0.03 μM ArgRS, 0.38 μM AsnRS, 0.13 μM AspRS, 0.02 μM CysRS, 0.06 μM GlnRS, 0.23 μM GluRS, 0.09 μM GlyRS, 0.02 μM HisRS, 0.4 μM IleRS, 0.04 μM LeuRS, 0.11 μM LysRS, 0.03 μM MetRS, 0.68 μM PheRS, 0.16 μM ProRS, 0.04 μM SerRS, 0.09 μM ThrRS, 0.03 μM TrpRS, 0.02 μM TyrRS and 0.02 μM VaIRS, 0.5 mM each of Ala, Arg, Asn, Asp, Cys, Gln, Glu, Gly, His, Ile, Leu, Lys, Met, Phe, Pro, Ser, Thr Trp, Tyr and Val, and 1.2 μM mRNA library conjugated to a puromycin linker. The in vitro translation was performed at 37° C. for 45 min in 150 μl (for the first round of selection) or 10 μl (from the second to fourth rounds) scale. The reaction mixture was incubated at room temperature for 12 min, and a 0.2× volume of 100 mM EDTA (pH 8.0) was added and incubated at 37° C. for 30 min to induce the dissociation of ribosomes from the mRNA-peptide conjugates. As previously described in Hipolito et al. (2013) Molecules, 18: 10514-10530; and Katoh et al. (2021) “In vitro selection of thioether-closed macrocycle peptide ligands by means of the RaPID system” in: Peptide Macrocycles: Methods and Protocols, vol 2371, Springer, New York, reverse transcription was carried out at 42° C. for 15 min using the CGS3an13.R22 primer and M-MLV reverse transcriptase lacking RNase H activity (Promega). Following reverse transcription, the mRNA:cDNA-peptide library was panned through a 2× translation volume of Dynabeads M-280 Streptavidin (Thermo Fisher, half-saturated with biotin) and incubated at 4° C. for 15 min, three times as a negative selection. Note that the negative selection was not performed at the first round. The supernatant retrieved from negative selection was added to 1× translation volume of Dynabeads immobilized with biotinylated human β-factor XIIa (Molecular Innovations, beads loading: 2 pmol protein/μL beads) and the mixture was incubated at 4° C. for 30 min (positive selection). The beads were washed with 100 μL of cold PBST (137 mM NaCl, 2.7 mM KCl, 10 mM Na₂HPO₄, 1.8 mM KH₂PO₄, 0.05% (v/v) Tween-20) three times and the cDNA was eluted from the beads by heating to 95° C. for 5 min in 100 μL of 1× PCR buffer (10 mM Tris-HCl (pH 9.0), 50 mM KCl, 0.1% (v/v) Triton X-100, 0.25 mM dNTP, 2.5 mM MgCl₂, 0.25 μM T7g10M.F46 and CGS3an13.R22 primers, and amplified by PCR. The elute (1 μL) was mixed with 19 μl of 1× PCR buffer that contained SYBR Green I and Taq DNA polymerase and the amount of cDNAs was quantified by real-time PCR. The rest elute was extracted by phenol/chloroform, precipitated by ethanol, dissolved in 10 μL water, and used for in vitro transcription of the subsequent round with the same recipe as preparation of the library. The scheme of an integrated round of selection is illustrated in FIG. 2.

### Solid Phase Peptide Synthesis

Peptides were synthesized using standard 9-fluorenylmethyl carbamate (Fmoc) solid-phase peptide synthesis chemistry as previously described in Cheneval et al. (2014) J Org Chem, 79: 5538-5544, the entire contents of which is incorporated herein by reference. In brief, each sequence was assembled on 2-chlorotrityl chloride resin (Chem-Impex, 0.45 mmol eq/g) using an automated peptide synthesizer (Symphony, Protein Technologies Inc.). Couplings were performed twice with 4 equiv of Fmoc-protected amino acids, 4 equiv of O-(6-chlorobenzotriazole-1-yl)-1,1,3,3-tetramethylaminium hexafluorophosphate (HCTU), and 8 equiv of N,N-diisopropylethylamine (DIPEA) in dimethylformamide (DMF) for 10 min. Removal of the Fmoc group was achieved using 30% piperidine in DMF (1 min). For the cyclic form of each sequence, side-chain protected peptides were cleaved from the solid support using several resin bed volume washes with 1% (v/v) trifluoracetic acid (TFA) in dichloromethane followed by lyophilization. Backbone cyclization was performed in DMF (50 mL per 0.1 mmol peptide) using 4 equiv of 1-[bis(dimethylamino)methylene]-1H-1,2,3-triazolo[4,5-b]pyridinium 3-oxid hexafluorophosphate (HATU) and 8 equiv of DIPEA for 6 h, followed by extraction and lyophilization. Cyclic precursor peptides were deprotected in a cocktail containing TFA/triisopropylsilane/water (95:2.5:2.5, v/v) and purified using RP-HPLC. Intramolecular disulfide bonds were formed in 0.1 M ammonium bicarbonate buffer (pH 8.5) by vigorous stirring at room temperature overnight. For the acyclic form of each sequence, peptides were cleaved from the solid support and the side chains were deprotected using a cleavage cocktail containing TFA/triisopropylsilane/water (95:2.5:2.5, v/v) for 2 h, followed by precipitation in diethyl ether and purification using RP-HPLC. Formation of intramolecular disulfide bonds was performed as described above. Purification of all peptides was performed on a Prominence HPLC system (Shimadzu) using a water:acetonitrile gradient (with 0.05% TFA). The purity (>95%) and integrity of the synthetic peptides were verified using LC-MS (LCMS-2020, Shimadzu).

For Examples 8 and 10, the peptides were synthesised as peptide hydrazides using solid phase synthesis to enable subsequent cyclization by intramolecular native chemical ligation. 2-chlorotrityl resin was swelled in DMF, then derivatized using 5% (v/v) NH₂NH₂ in DMF (3×30 min). After washing the resin with DMF, unreacted sites were capped using 10% (v/v) methanol (MeOH) in DMF (10 min). The first residue was coupled manually using 4 equiv. Fmoc-Na protected amino acid, 4 equiv. PyBOP and 4 equiv. DIPEA in DMF (2×30 min) and remaining residues were coupled by automated synthesis (Symphony, Protein Technologies Inc.) using the same method as described above. Crude peptides were cleaved from the resin and deprotected using TFA/triisopropylsilane/H₂O (96:2:2) before purification by HPLC as described above. To generate peptide thioesters, purified peptide hydrazides (3 mM) were dissolved in 6 M guanidine hydrochloride pH 2.5 containing 3 equiv. acetylacetone and 200 mM 4-mercaptophenylacetic acid. After stirring for 4 h, peptides were diluted to 0.5 mM using 0.1 M phosphate buffer containing 6 M guanidine hydrochloride and 50 mM tris(2-carboxyethyl)phosphine (TCEP), and the pH adjusted to 7. The cyclization reaction proceeded overnight with stirring. Cyclic peptides were purified, then subjected to oxidative folding again as described above.

### ¹H NMR Spectroscopic Characterization

Lyophilized peptides (purity>95%) were dissolved in 90% H₂O/10% D₂O (v/v) to approximately 1 mM. ¹H one- and two-dimensional TOCSY (total correlation spectroscopy) and NOESY (nuclear Overhauser effect spectroscopy) spectra of MCoTI-II, MCoFx1-5 in both acyclic and cyclic forms, cMCoTI-fxL1, and cMCoTI-fxL5 were acquired using an Avance-600 MHz spectrometer (Bruker) at 25° C. The mixing time was 80 ms and 200 ms for TOCSY and NOESY, respectively. Spectra were internally referenced to 2,2-dimethyl-2-silapentane-5-sulfonic acid (DSS) at 0.00 ppm and analyzed using CcpNMR Analysis V2. The α-proton secondary chemical shifts of peptides were calculated by subtracting random coil chemical shifts as reported previously in Wishart et al. (1995) J Biomol NMR, 6: 135-140, from the experimentally observed shifts.

### Surface Plasmon Resonance for Binding Affinity Determination

Binding kinetics of each peptide towards biotinylated human β-FXIIa (Molecular Innovations) and non-labelled zymogen FXII (Haematologic Technologies) were determined using a Biacore T200 machine (Cytiva). For β-FXIIa binding measurements, the running buffer was HBS-EP+(10 mM HEPES, 150 mM NaCl, 3 mM EDTA and 0.05% (v/v) surfactant P20, pH 7.4). Biotinylated β-FXIIa was immobilized on a Sensor Chip CAP (Cytiva) using Biotin CAPture Reagent (Cytiva). A series of concentrations of each peptide were injected as analyte and the binding kinetics were modelled using a 1:1 binding model. Regeneration of sensor surface was performed after each measurement cycle. For FXII binding measurements, the running buffer was PBST (137 mM NaCl, 2.7 mM KCl, 10 mM Na₂HPO₄, 1.8 mM KH₂PO₄, 0.05% Tween-20). Non-labelled FXII was immobilized on a Sensor Chip CM5 (Cytiva) using Amine Coupling Kit (Cytiva), achieving a final immobilization level of 2500 RU. Each peptide was flowed through immobilized FXII at 5 μM for 60 s and allowed dissociation for 150 s.

### Protease Inhibition Assays

The activity of acyclic and cyclic MCoFx1-5 and analogues thereof was assessed in competitive inhibition assays, as previously described in Swedberg et al. (2016) J Med Chem, 59: 7287-7292. Human β-FXIIa was obtained from Molecular Innovations, together with human FXa, human FXIa, human α-thrombin, and human plasma kallikrein. Human cationic trypsin and human plasmin were obtained from Sigma-Aldrich, and recombinant human matriptase was sourced from R&D Systems. Recombinant human urokinase (uPA) and recombinant human tissue plasminogen activator (tPA) were expressed in Expi293 cells, as previously described in Li et al. (2019) Chembiochem, 20: 46-50. FXIIa inhibition assays were performed in clear, low-binding 96-well plates using 250 μL (final volume) of assay buffer (0.1 M Tris-HCl pH 8.0, 0.1 M NaCl, 10 mM CaCl₂ and 0.005% Triton X-100). Inhibitors were serially diluted and incubated with 10 nM FXIIa at room temperature to reach equilibrium. After adding 100 μM Ac-QRFR-pNA substrate (K_{M}=162 μM), enzymatic activity was measured by monitoring release of the pNA moiety using a TECAN infinite M1000 Pro plate reader (A=405 nm, reading interval, 10 s; assay time course, 7 min). Assays were conducted in three times in duplicate. Substrate kinetic constants (Michaelis-Menten) and inhibition constants (Morrison K) were determined by non-linear regression using Prism 7 (GraphPad). Selectivity assays with off-target proteases were performed in a similar way, except that different concentrations of enzyme and substrate were used, as indicated in Table 16. For assays using a fluorescent substrate (peptide-4-methylcoumaryl-7-amide, MCA), black low-binding 96-well plates were used and activity was measured using a TECAN infinite M1000 pro plate reader (λₑₓ 360 nm, λₑₘ 460 nm, reading interval, 30 s; assay time course, 5-30 min).

TABLE 16
PROTEASE-SPECIFIC CONDITIONS AND SUBSTRATES
FOR INHIBITION ACTIVITY EXPERIMENTS

| | | | | | | | | Buffer |
|---|---|---|---|---|---|---|---|---|
| Enzymeª | Conc. | | Substrate | Conc. | | K_{M} | | variation^{b} |
| Human β-FXIIa | 10 | nM^{c} | Ac-QRFR-pNA | 100 | μM | 162 | μM | 10 mM CaCl₂ |
| Human β-FXIIa | 0.5 | nM | Boc-QGR-MCA | 50 | μM | 187 | μM | 10 mM CaCl₂ |
| Human trypsin | 0.1 | nM | Boc-VPR-MCA | 5 | μM | 3.8 | μM | 10 mM CaCl₂ |
| Human FXa | 0.2 | nM | Z-Pyr-GR-MCA | 50 | μM | — | | 10 mM CaCl₂ |
| Human FXIa | 1 | nM | Boc-Glu(OBzl)-AR- | 100 | μM | — | | 10 mM CaCl₂ |
| | | | MCA | | | | | |
| Human thrombin | 0.1 | nM | Boc-VPR-MCA | 40 | μM | — | | 10 mM CaCl₂ |
| Human plasma kallikrein | 0.5 | nM | Z-FR-MCA | 50 | μM | — | | — |
| Human plasmin | 1 | nM | Ac-RM(O2)YR-pNA | 75 | μM | 24 | μM | — |
| Rec. human matriptase | 0.15 | nM | Boc-QAR-MCA | 10 | μM | 42 | μM | — |

| | | | | | | | |
|---|---|---|---|---|---|---|---|
| Rec. human uPA | 10 | nM | Z-Pyr-GR-MCA | 100 | μM | — | 10 mM CaCl₂ |
| Rec. human tPA | 10 | nM | Z-Pyr-GR-MCA | 100 | μM | — | 10 mM CaCl₂ |

ªEnzymes purified from human plasma, human tissue (trypsin) or recombinantly expressed (indicated by rec.)
^{b}Assay buffer was 0.1 M Tris-HCl pH 8.0, 0.1 M NaCl, 0.005% Triton X100 with 10 mM CaCl₂ included for the indicated enzymes
^{c}Assays with high-affinity inhibitors (MCoFx1 and MCoFx1-L1) were performed with 4.2 nM enzyme

### Serum Stability

To provide a guide for their potential in vivo stability, the proteolytic stability of acyclic MCoFx1, cMCoFx1, cMCoTI-fxL1 and the parent peptide MCoTI-II was evaluated in human serum as described previously in Huang et al. (2015) Sci Rep, 5: 12974. Briefly, peptides were incubated at a final concentration of 30 μM in human serum (human male AB serum, Sigma-Aldrich) at 37° C. for 0, 1, 12 and 24 h. The reaction was stopped at designated times, with the serum proteins being denatured by addition of two volumes of acetonitrile (v/v). The samples were then spun at 17,000 g for 10 min and the amount of peptide in the supernatant was quantified using analytical HPLC. The percentage of peptide remaining at 1, 12 and 24 h was calculated using the area of the serum-treated peptide peak from 0 h as 100%. A linear peptide (EAIYAAPFAKKK) was included as a control to evaluate the proteolytic activity of human serum, which was rapidly degraded (within 1 h) in serum that had been incubated at 37° C. for 0, 4, or 24 h.

### Coagulation Assays

Citrated, platelet-poor plasma samples from healthy adults were collected, prepared, and stored using previously established collection protocols, such as in Zdenek et al. (2019) Toxicol in Vitro, 58: 97-109.

Standard coagulation assays were used to determine the effect of the inhibitors on the clotting time of human plasma. Activated partial thromboplastin time (aPTT) uses a Kaolin reagent (clay) to activate the intrinsic/contact pathway of the coagulation cascade, whereas prothrombin time (PT) uses Neoplastine (lyophilized thromboplastin prepared from rabbit cerebral tissue) to activate the extrinsic/tissue-factor pathway of the coagulation cascade. A minor adjustment was made to both tests (addition of 25 μL inhibitor or buffer; refer to Table 17) to accommodate the addition of an inhibitor without changing the total volume (and therefore relative ratios of additives) of the assay. Concentrations of inhibitor are reported as the concentration at the point of incubation in the assay, i.e. not taking into account the addition of the start reagent (calcium for aPTT, or Neoplastine for PT).

TABLE 17
EXPERIMENTAL PROCEDURES FOR APTT AND PT ASSAYS

| Experiment | Methodology |
|---|---|
| aPTT* | Step 1: 25 μL inhibitor (solubilized in Owren-Koller |
| | (OK) Buffer (isotonic saline, Stago #00360)) + 50 μL |
| | kaolin/phospholipid (Stago #00597) + 50 μL human plasma |
| | Step 2: 240 s incubation at 37° C. |
| | Step 3: Addition of 50 μL 0.025 M calcium (Stago #00367) |
| PT* | Step 1: 25 μL inhibitor (diluted with OK Buffer) + 50 μL |
| | human plasma |
| | Step 2: 240 s incubation at 37° C. |
| | Step 3: Addition of 100 μL Neoplastine (Stago #00606) |

*Control assays replaced 25 μL inhibitor with 25 μL OK buffer.
aPTT = Activated Partial Thromboplastin Time;
PT = Prothrombin Time.

Clotting time (seconds) of plasma was automatically measured using a STA-R Max® analyzer (Stago, Asnieres sur Seine, France). Measurements were conducted using a viscosity-based (mechanical) detection system, whereby opposing magnets oscillate a small metal spherical pellet inside the test cuvette (250 μL total volume) until a clot is formed. Dilution of the inhibitor in Owren-Koller (OK) buffer for dose-response curves was performed automatically by the machine. Reagents were kept at 15-19° C. in the machine during experimentation and otherwise stored at 4° C. All tests were performed in triplicate.

### Cytotoxicity Assay

The cytotoxicity of cMCoFx1 was assessed as described previously (Huang et al. (2015), Sci Rep, 5:12974) on human umbilical vein endothelial cells (HUVECs). HUVECs were cultured in complete EGM™-2 medium until 80% confluence under 5% CO₂ at 37° C. before being seeded onto a 96-well microplate at 5,000 cells/well. Cells were grown for 24 h prior to the assay. Peptide stocks of cMCoFx1 and MCoTI-II were prepared in ultrapure H₂O at 640 μM and then serially diluted by two-fold dilution (640-10 μM). Peptides were diluted tenfold with serum-free EGM™-2 medium and incubated with HUVECs in triplicate. After 24 h incubation, peptide solutions were replaced with fresh complete EGM™-2 medium before the addition of 0.05% resazurin solution (Sigma-Aldrich). Cells were incubated for another 8 h and the absorbance of the plate was measured on a microplate reader at 540 and 620 nm. H₂O and 1% Triton-X were used as control for 100 and 0% viability, separately.

### ECMO Experiments

Ex vivo ECMO circuits (Permanent Life Support [PLS] System, Maquet C P, Rastatt, Germany) consisted of a Quadrox D Oxygenator and ROTAFLOW centrifugal pump, both incorporated into a tubing set, with a tip-to-tip BIOLINE (albumin and heparin) coating. Initially, the PLS system was primed with Plasma-Lyte 148 and heated to 37.8° C. Donated healthy human whole blood was collected into top-and-bottom bags with a built-in filter, containing citrate phosphate dextrose. Prior to loading on the circuit, blood was prepared by adding NaHCO₃, CaCl₂), and Plasma-Lyte (10 mL), followed by either heparin or cyclic MCoFx7. Heparin (350 UI) was added at 0 h, followed by 10 UI at 1 h, 20 UI at 2 h, 10 UI at 3 h, and 10 UI at 4 h. For cyclic MCoFx7, a single dose (20 μM) was added at 0 h. Hematocrit levels were checked and adjusted to 34±2% if necessary. Blood was subsequently introduced into the circuit (total blood volume in the circuit, 450±20 mL). Once blood flow had stabilized, the flow rate was adjusted to 4 L/min. Inlet and outlet pressures between the membrane oxygenator were monitored using a silicone-based pressure transducer (Omega Engineering, Norwalk, CT, USA) and were recorded as the delta oxygenator pressure (P). Blood samples were taken at 30 min, 2 h, 4 h, and 6 h (the volume taken was replaced with an equivalent volume of Plasma-Lyte).

### ACT and ROTEM Assays

Activated clotting time (ACT) was measured in fresh whole blood using celite tubes with the Hemochron 401 coagulation analyzer (Soma Technology, Bloomfield, CT, USA). Whole blood clot formation was recorded by ROTEM® Thromboelastometry (Haemoview Diagnostics, Brisbane, Australia) using INTEM (contact factor-initiated coagulation) and HEPTEM (contact factor-initiated coagulation with heparinase) activating reagents according to the manufacturer's instructions.

### Library Generation for Saturation Mutagenesis Study

The saturation mutagenesis library was designed based on the mRNA display selected sequence, MCoFx1 (MDGGICPRIGRLCRRDSDCPGACICRATRFCGSGSGS [SEQ ID NO: 98]), with a random residue replacing the parental residue at the core positions (DGGICPRIGRLCRRDSDCPGACICRATRFCGSGS [SEQ ID NO: 99]) in each mutant sequence. Oligonuclotides corresponding to the parental MCoFx1 and single-position mutants were ordered from Eurofins Genomics, with the random residue encoding with NNK codon (N=A, T, G, C and K=G, T) (refer to Table 18). The DNA library was constructed in a two-step PCR reaction using home-made KOD polymerase, with the first extension step extending two pieces of oligos, MCoFx1-reg1_original.F89 or MCoFx1-reg1_pn.F89 and MCoFx1.R70 for n=1-16, or MCoFx1.F72 and MCoFx1-reg2_pn.R87 for n=17-34. The second amplification PCR added T7g10M.F48 and Long_HA.R63 primers, with the product containing upstream T7 promoter, GGG triplet, epsilon sequence and ribosome binding (Shine-Dalgarno) sequence and downstream HA tag and puromycin linker binding sequence. For each parent or single-position mutated template, the PCR reaction was conducted in 100 mM Tris-HCl (pH 8.0), 6 mM (NH₄)₂SO₄, 10 mM KCl, 1% (v/v) Triton X-100, 0.25 mM dNTP, 2.5 mM MgCl₂, 0.5 μM forward and reverse primers, and 1% (v/v) 1× home-made KOD polymerase, and the PCR conditions used are listed in Table 19. The first-step extension PCR was conducted in 10 μL scale and added to 100 μL of the second-step amplification PCR reaction. The products were extracted by phenol/chloroform, precipitated by ethanol, dissolved in 20 μL water, and used for in vitro transcription at 37° C. for 16 h in a 200 μL reaction scale. The in vitro transcription reaction mixture contained 40 mM Tris-HCl, 1 mM spermidine, 0.01% (v/v) Triton X-100, 10 mM DTT, 30 mM MgCl₂, 5 mM NTPs, 30 mM KOH, 650 μL template DNA solution, 0.12 μM home-made T7 RNA polymerase at pH 8.0. The resulting mRNA transcripts were precipitated by adding 10% (v/v) 3M NaCl and 80% (v/v) of isopropanol followed by centrifuge. After wash with 70% (v/v) EtOH, the pellet was dissolved in water and the concentration was adjusted to 10 μM. The parent and single-position mutated mRNA templates were equally mixed, yielding a 10 μM mRNA library for the following target scanning.

TABLE 18
SEQUENCES OF OLIGONUCLEOTIDES USED FOR LIBRARY ASSEMBLY, REVERSE TRANSCRIPTION
AND PCR REACTIONS IN SATURATION MUTAGENESIS STUDY

Common

Oligonucleotides for saturation mutagenesis library generation

| | |
|---|---|
| T7g10M.F48 | TAATACGACTCACTATAGGGTTAACTTTAAGAAGGAGATATACATATG [SEQ ID NO: 107] |
| Long_HA.R63 | TTTCCGCCCCCCGTCCTAAGAACCAGAACCAGAACCTGCATAGTCGGGCACGTCGTATGGGTA |
| | [SEQ ID NO: 108] |
| Short_HA.R36 | TTTCCGCCCCCCGTCCTAAGAACCAGAACCAGAACC [SEQ ID NO: 109] |
| MCo_HA_Rd1T7g10M. | CACTCTTTCCCTACACGACGCTCTTCCGATCTAACTITAAGAAGGAGATATACAT [SEQ ID NO: |
| F55 | 110] |
| MCo_HA_Rd2R49c. | GACTGGAGTTCAGACGTGTGCTCTTCCGATCTAGTCGGGCACGTCGTATGGGTA [SEQ ID NO: |
| R54 | 111] |

For parental MCoFx1 and reg1_p1-16 templates

| | |
|---|---|
| MCoFx1- | CTTTAAGAAGGAGATATACATATGGATGGTGGCATTTGCCCGCGGATTGGGCGGCTTTGCCGT |
| reg1_original.F89 | CGTGATTCTGACTGCCCCGGTGCGTG [SEQ ID NO: 112] |
| MCoFx1- | CTTTAAGAAGGAGATATACATATGNNKGGTGGCATTTGCCCGCGGATTGGGCGGCTTTGCCGT |
| reg1_p1.F89 | CGTGATTCTGACTGCCCCGGTGCGTG [SEQ ID NO: 113] |
| MCoFx1- | CTTTAAGAAGGAGATATACATATGGATNNKGGCATTTGCCCGCGGATTGGGCGGCTTTGCCGT |
| reg1_p2.F89 | CGTGATTCTGACTGCCCCGGTGCGTG [SEQ ID NO: 114] |
| MCoFx1- | CTTTAAGAAGGAGATATACATATGGATGGTNNKATTTGCCCGCGGATTGGGCGGCTTTGCCGTC |
| reg1_p3.F89 | GTGATTCTGACTGCCCCGGTGCGTG [SEQ ID NO: 115] |
| MCoFx1- | CTTTAAGAAGGAGATATACATATGGATGGTGGCNNKTGCCCGCGGATTGGGCGGCTTTGCCGT |
| reg1_p4.F89 | CGTGATTCTGACTGCCCCGGTGCGTG [SEQ ID NO: 116] |
| MCoFx1- | CTTTAAGAAGGAGATATACATATGGATGGTGGCATTNNKCCGCGGATTGGGCGGCTTTGCCGT |
| reg1_p5.F89 | CGTGATTCTGACTGCCCCGGTGCGTG [SEQ ID NO: 117] |
| MCoFx1- | CTTTAAGAAGGAGATATACATATGGATGGTGGCATTTGCNNKCGGATTGGGCGGCTTTGCCGT |
| reg1_p6.F89 | CGTGATTCTGACTGCCCCGGTGCGTG [SEQ ID NO: 118] |
| MCoFx1- | CTTTAAGAAGGAGATATACATATGGATGGTGGCATTTGCCCGNNKATTGGGCGGCTTTGCCGTC |
| reg1_p7.F89 | GTGATTCTGACTGCCCCGGTGCGTG [SEQ ID NO: 119] |
| MCoFx1- | CTTTAAGAAGGAGATATACATATGGATGGTGGCATTTGCCCGCGGNNKGGGCGGCTTTGCCGT |
| reg1_p8.F89 | CGTGATTCTGACTGCCCCGGTGCGTG [SEQ ID NO: 120] |
| MCoFx1- | CTTTAAGAAGGAGATATACATATGGATGGTGGCATTTGCCCGCGGATTNNKCGGCTTTGCCGTC |
| reg1_p9.F89 | GTGATTCTGACTGCCCCGGTGCGTG [SEQ ID NO: 121] |
| MCoFx1- | CTTTAAGAAGGAGATATACATATGGATGGTGGCATTTGCCCGCGGATTGGGNNKCTTTGCCGTC |
| reg1_p10.F89 | GTGATTCTGACTGCCCCGGTGCGTG [SEQ ID NO: 122] |
| MCoFx1- | CTTTAAGAAGGAGATATACATATGGATGGTGGCATTTGCCCGCGGATTGGGCGGNNKTGCCGT |
| reg1_p11.F89 | CGTGATTCTGACTGCCCCGGTGCGTG [SEQ ID NO: 123] |
| MCoFx1- | CTTTAAGAAGGAGATATACATATGGATGGTGGCATTTGCCCGCGGATTGGGCGGCTTNNKCGT |
| reg1_p12.F89 | CGTGATTCTGACTGCCCCGGTGCGTG [SEQ ID NO: 124] |
| MCoFx1- | CTTTAAGAAGGAGATATACATATGGATGGTGGCATTTGCCCGCGGATTGGGCGGCTTTGCNNK |
| reg1_p13.F89 | CGTGATTCTGACTGCCCCGGTGCGTG [SEQ ID NO: 125] |
| MCoFx1- | CTTTAAGAAGGAGATATACATATGGATGGTGGCATTTGCCCGCGGATTGGGCGGCTTTGCCGT |
| reg1_p14.F89 | NNKGATTCTGACTGCCCCGGTGCGTG [SEQ ID NO: 126] |
| MCoFx1- | CTTTAAGAAGGAGATATACATATGGATGGTGGCATTTGCCCGCGGATTGGGCGGCTTTGCCGT |
| reg1_p15.F89 | CGTNNKTCTGACTGCCCCGGTGCGTG [SEQ ID NO: 127] |
| MCoFx1- | CTTTAAGAAGGAGATATACATATGGATGGTGGCATTTGCCCGCGGATTGGGCGGCTTTGCCGT |
| reg1_p16.F89 | CGTGATNNKGACTGCCCCGGTGCGTG [SEQ ID NO: 128] |
| MCoFx1.R70 | GCACGTCGTATGGGTAGCTGCCGCTGCCGCAAAACCTAGTAGCCCTGCAAATGCACGCACCGG |
| | GGCAGTC [SEQ ID NO: 129] |

For reg2_p17-34 templates

| | |
|---|---|
| MCoFx1.F72 | CTTTAAGAAGGAGATATACATATGGATGGTGGCATTTGCCCGCGGATTGGGCGGCTTTGCCGT |
| | CGTGATTCT [SEQ ID NO: 130] |
| MCoFx1- | GCACGTCGTATGGGTAGCTGCCGCTGCCGCAAAACCTAGTAGCCCTGCAAATGCACGCACCGG |
| reg2_p17.R87 | GGCAMNNAGAATCACGACGGCAAA [SEQ ID NO: 131] |
| MCoFx1- | GCACGTCGTATGGGTAGCTGCCGCTGCCGCAAAACCTAGTAGCCCTGCAAATGCACGCACCGG |
| reg2_p18.R87 | GMNNGTCAGAATCACGACGGCAAA [SEQ ID NO: 132] |
| MCoFx1- | GCACGTCGTATGGGTAGCTGCCGCTGCCGCAAAACCTAGTAGCCCTGCAAATGCACGCACCMN |
| reg2_p19.R87 | NGCAGTCAGAATCACGACGGCAAA [SEQ ID NO: 133] |
| MCoFx1- | GCACGTCGTATGGGTAGCTGCCGCTGCCGCAAAACCTAGTAGCCCTGCAAATGCACGCMNNGG |
| reg2_p20.R87 | GGCAGTCAGAATCACGACGGCAAA [SEQ ID NO: 134] |
| MCoFx1- | GCACGTCGTATGGGTAGCTGCCGCTGCCGCAAAACCTAGTAGCCCTGCAAATGCAMNNACCGG |
| reg2_p21.R87 | GGCAGTCAGAATCACGACGGCAAA [SEQ ID NO: 135] |
| MCoFx1- | GCACGTCGTATGGGTAGCTGCCGCTGCCGCAAAACCTAGTAGCCCTGCAAATMNNCGCACCGG |
| reg2_p22.R87 | GGCAGTCAGAATCACGACGGCAAA [SEQ ID NO: 136] |
| MCoFx1- | GCACGTCGTATGGGTAGCTGCCGCTGCCGCAAAACCTAGTAGCCCTGCAMNNGCACGCACCG |
| reg2_p23.R87 | GGGCAGTCAGAATCACGACGGCAAA [SEQ ID NO: 137] |
| MCoFx1- | GCACGTCGTATGGGTAGCTGCCGCTGCCGCAAAACCTAGTAGCCCTMNNAATGCACGCACCGG |
| reg2_p24.R87 | GGCAGTCAGAATCACGACGGCAAA [SEQ ID NO: 138] |
| MCoFx1- | GCACGTCGTATGGGTAGCTGCCGCTGCCGCAAAACCTAGTAGCMNNGCAAATGCACGCACCG |
| reg2_p25.R87 | GGGCAGTCAGAATCACGACGGCAAA [SEQ ID NO: 139] |
| MCoFx1- | GCACGTCGTATGGGTAGCTGCCGCTGCCGCAAAACCTAGTMNNCCTGCAAATGCACGCACCGG |
| reg2_p26.R87 | GGCAGTCAGAATCACGACGGCAAA [SEQ ID NO: 140] |
| MCoFx1- | GCACGTCGTATGGGTAGCTGCCGCTGCCGCAAAACCTMNNAGCCCTGCAAATGCACGCACCGG |
| reg2_p27.R87 | GGCAGTCAGAATCACGACGGCAAA [SEQ ID NO: 141] |
| MCoFx1- | GCACGTCGTATGGGTAGCTGCCGCTGCCGCAAAAMNNAGTAGCCCTGCAAATGCACGCACCG |
| reg2_p28.R87 | GGGCAGTCAGAATCACGACGGCAAA [SEQ ID NO: 142] |
| MCoFx1- | GCACGTCGTATGGGTAGCTGCCGCTGCCGCAMNNCCTAGTAGCCCTGCAAATGCACGCACCG |
| reg2_p29.R87 | GGGCAGTCAGAATCACGACGGCAAA [SEQ ID NO: 143] |
| MCoFx1- | GCACGTCGTATGGGTAGCTGCCGCTGCCMNNAAACCTAGTAGCCCTGCAAATGCACGCACCGG |
| reg2_p30.R87 | GGCAGTCAGAATCACGACGGCAAA [SEQ ID NO: 144] |
| MCoFx1- | GCACGTCGTATGGGTAGCTGCCGCTMNNGCAAAACCTAGTAGCCCTGCAAATGCACGCACCGG |
| reg2_p31.R87 | GGCAGTCAGAATCACGACGGCAAA [SEQ ID NO: 145] |
| MCoFx1- | GCACGTCGTATGGGTAGCTGCCMNNGCCGCAAAACCTAGTAGCCCTGCAAATGCACGCACCGG |
| reg2_p32.R87 | GGCAGTCAGAATCACGACGGCAAA [SEQ ID NO: 146] |
| MCoFx1- | GCACGTCGTATGGGTAGCTMNNGCTGCCGCAAAACCTAGTAGCCCTGCAAATGCACGCACCGG |
| reg2_p33.R87 | GGCAGTCAGAATCACGACGGCAAA [SEQ ID NO: 147] |
| MCoFx1- | GCACGTCGTATGGGTAMNNGCCGCTGCCGCAAAACCTAGTAGCCCTGCAAATGCACGCACCGG |
| reg2_p34.R87 | GGCAGTCAGAATCACGACGGCAAA [SEQ ID NO: 148] |

TABLE 19
PCR REACTION CONDITIONS
FOR SATURATION
MUTAGENESIS
LIBRARY ASSEMBLY
Extension PCR

| | | | | |
|---|---|---|---|---|
| 94° C. | 1 | min | 1 | cycle |
| 52° C. | 1 | min | 5 | cycles |
| 68° C. | 1 | min | | |

Amplification PCR

| | | | | |
|---|---|---|---|---|
| 94° C. | 40 | s | 12 | cycles |
| 50° C. | 40 | s | | |
| 68° C. | 40 | s | | |

### Translation and Reverse Transcription

The mRNA library was covalently linked to a puromycin linker (refer to Table 4) using home-made T4 RNA ligase. The reaction containing 1 μM mRNA, 1.5 μM puromycin linker and 1 μM T4 ligase in ligation buffer (40 mM Tris, pH 7.8, 10 mM MgCl₂, 10 mM DTT, 0.5 mM ATP) was incubated at 25° C. for 30 min. Ligated mRNA was extracted with phenol/chloroform, precipitated with ethanol, washed with 70% (v/v) ethanol, and redissolved in water. Success of puromycin ligation was judged by 8% polyacrylamide gel electrophoresis. Ligation product was diluted with water to 5 μM and frozen at −20° C. for storage. In vitro translation of the Pu-ligated mRNA library was conducted in a translation cocktail as previously described in Goto et al. (2011) Nat Protoc, 6: 779-790. In brief, the translation mixture consisted of 50 mM HEPES-KOH (pH 7.6), 100 mM potassium acetate, 12.3 mM magnesium acetate, 2 mM ATP, 2 mM GTP, 1 mM CTP, 1 mM UTP, 20 mM creatine phosphate, 2 mM spermidine, 1 mM dithiothreitol, 100 μM 10-formyl-5,6,7,8-tetrahydrofolic acid, 1.5 mg ml⁻¹ E. coli total tRNA, 1.2 μM E. coli ribosome, 0.6 μM methionyl-tRNA formyltransferase, 2.7 μM IF1, 0.4 μM IF2, 1.5 μM IF3, 0.26 μM EF-G, 10 μM EF-Tu/EF-Ts complex, 0.25 μM RF2, 0.17 μM RF3, 0.5 μM RRF, 4 μg ml⁻¹ creatine kinase, 3 μg ml⁻¹ myokinase, 0.1 μM inorganic pyrophosphatase, 0.1 μM nucleotide diphosphate kinase, 0.1 μM T7 RNA polymerase, 0.73 μM AlaRS, 0.03 μM ArgRS, 0.38 μM AsnRS, 0.13 μM AspRS, 0.02 μM CysRS, 0.06 μM GlnRS, 0.23 μM GluRS, 0.09 μM GlyRS, 0.02 μM HisRS, 0.4 μM IleRS, 0.04 μM LeuRS, 0.11 μM LysRS, 0.03 μM MetRS, 0.68 μM PheRS, 0.16 μM ProRS, 0.04 μM SerRS, 0.09 μM ThrRS, 0.03 μM TrpRS, 0.02 μM TyrRS and 0.02 μM VaIRS, 0.5 mM each of Ala, Arg, Asn, Asp, Cys, Gln, Glu, Gly, His, Ile, Leu, Lys, Met, Phe, Pro, Ser, Thr Trp, Tyr and Val, and 1.2 μM mRNA library conjugated to a puromycin linker. The in vitro translation was performed at 37° C. for 45 min in 5 μl scale. The reaction mixture was incubated at room temperature for 12 min, and a 0.2× volume of 100 mM EDTA (pH 8.0) was added and incubated at 37° C. for 30 min to induce the dissociation of ribosomes from the mRNA-peptide conjugates. Reverse transcription was carried out at 42° C. for 60 min using Short_HA.R36 primer and M-MLV reverse transcriptase lacking RNase H activity (Promega).

### HA Affinity Purification

To 10 μL of reverse transcription product, 1× volume of 2× blocking buffer was added (2× PBST supplemented with 2 mg/mL bovine serum albumin, where 2× PBST contained 274 mM NaCl, 5.4 mM KCl, 20 mM Na₂HPO₄, 3.6 mM of KH₂PO₄, and 0.1% (v/v) Tween-20). 20 μl of 10 mg/mL Anti-HA beads (Thermo Fisher) were washed twice with PBST before addition of the reverse transcription mixture. Incubation at 4° C. for 60 min ensued, after which the supernatant was discarded and the beads were washed twice with PBST. Bound mRNA:cDNA-peptide conjugates were eluted from the beads with HA peptide (2 mg/mL in blocking buffer; sequence: NH₂-YPYDVPDYA-CONH₂) by incubating the suspension at 37° C. for 15 min, and collecting the supernatant. HA affinity purification was indispensable to remove peptide-unconjugated, puromycin-unligated mRNA/cDNA, and other translation side-products.

Separation into Non-Binding and Binding Fractions

To the HA-purified mRNA:cDNA-peptide library, 1× translation volume of Dynabeads immobilized with biotinylated human β-factor XIIa (Molecular Innovations, beads loading: 2 pmol protein/μl beads) was added and the mixture was incubated at 25° C. for 3 h, allowing the binding reaction to reach equilibrium. The supernatant was retrieved and stored as “non-binding sample” at −20° C. The beads were washed twice using 1× blocking buffer at 4° C. for 16 h and 25° C. for 3 h. Elution of the “binding” cDNA was carried out by heating the beads suspended in 0.1% (v/v) Triton X-100 at 95° C. for 5 min.

### Sample Preparation for Next Generation Sequencing (NGS)

Concentrations of recovered “binding” cDNA, as well as the “non-binding” cDNA were determined from a qPCR assay. Recovered sample aliquots were analyzed by qPCR with Taq polymerase in 10 mM Tris-HCl (pH 9.0), 50 mM KCl, 0.1% (v/v) Triton X-100, 0.25 mM dNTP, 2.5 mM MgCl₂, 0.25 μM T7g10M.F48 and Short_HA.R36 primers, and home-made Taq polymerase.

Recovered “binding” cDNA, as well as the “non-binding” cDNA were PCR-amplified with Platinum SuperFi DNA Polymerase (Thermo Fisher) using manufacturer's protocol and 0.25 μM MCo_HA_Rd1T7g10M.F55 and MCo_HA_Rd2R49c.R54 primers (refer to Table 18). Thermal cycling was performed based on the outcomes of qPCR so as to avoid cDNA overamplification. The product was carried forward to the second PCR step, using SuperFi Polymerase and Nextera XT v2 Set (sequences from Illumina) primers to install sequencing barcodes on each sample. The success of PCR was confirmed by 3% agarose gel electrophoresis. After, PCR products were combined and column-purified using a NucleoSpin kit (TaKaRa) adhering to the manufacturer's protocol. Concentration of the combined cDNA sample was measured with Qubit (Thermo Fisher) using the dsDNA BR kit. The resulting DNA was appropriately diluted and analyzed by NGS.

Sequencing with NGS and Data Analysis

Denatured cDNA library (10 μM containing 20% (mol/mol) PhiX Control v3 (Illumina) was sequenced on Illumina's MiSeq instrument in the single read 1×151 cycle mode using v3 chip, collecting data in the .fastq format. The details of the downstream data analysis can be found at https://github.com/avngrdv/FastqProcessor. Briefly, .fastq data files containing base calls were parsed to retrieve DNA sequences, which were in silico translated. Resulting peptide lists were filtered to discard sequences containing ambiguous symbols or not conforming to the library design criteria (overall peptide length). Constant region sequences were trimmed and the variable regions were further filtered to discard double or poly-mutants. The resulting peptides comprised the final data set for each sample. With these data sets, frequencies for each mutant were computed, and then “binding” and “non-binding” frequency matrices were compared to calculate W-scores of each mutant. The W-score is defined as below:

Define fₘᵤₜ, i.e. frequency of an individual mutant mut in a sequencing sample as

$${f}_{mut} = \frac{{c}_{mut}}{{c}_{total}}$$

where cₘᵤₜ is the number of reads corresponding to mutant mut, and cₜₒₜₐₗ is the total number of reads in the sample.

Then, Y-score for mutant mut is defined as

$${Y}_{mut} = \frac{{f}_{mut} ( binding sample )}{{f}_{mut} ( non - binding sample )}$$

To compare the binding trend of mutants compared with the parental MCoFx1, W-score for mutant mut is defined as

$${W}_{mut} = {log}_{2} ( {Y}_{mut} ) - {log}_{2} ( {Y}_{MCoFx 1} )$$

where Y_{MCoFx1} is the Y-score corresponding to parental sequence MCoFx1.

Based on the definition, W-score of the parental MCoFx1 is 0; for mutant mut with Wₘᵤₜ>0, the mutation is beneficial for target-binding compared with MCoFx1; for mutant mut with Wₘᵤₜ<0, the mutation is disadvantageous for target-binding compared with MCoFx1.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### EMBODIMENTS

Exemplary embodiments include, but are not limited to:

1. A proteinaceous molecule comprising an amino acid sequence represented by Formula I:

CX₁X₂X₃X₄X₅X₆CX₇X₈DSDCPGACICX₉X₁₀X₁₁X₁₂X₁₃C (I)

wherein:
X₁ is selected from P and modified forms thereof; C and modified forms thereof; and F and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; hydrophobic amino acid residues including V, L, I, Ne and modified forms thereof; and acidic amino acid residues including D, E, hGlu and modified forms thereof;
X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; basic amino acid residues including K, R, H and modified forms thereof; and amide containing amino acid residues including N, Q and modified forms thereof;
X₆ is selected from any amino acid residue;
X₇ is selected from basic amino acid residues including K, R, H and modified forms thereof; amide containing amino acid residues including N, Q and modified forms thereof; and small amino acid residues including S, T, A, G and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof; wherein the proteinaceous molecule is other than a proteinaceous molecule comprising or consisting of the amino acid sequence of any one of SEQ ID NOs: 1 to 7:

[SEQ ID NO: 1]
CPKILKKCRRDSDCPGACICRGNGYC;
[SEQ ID NO: 2]
CPKILQRCRRDSDCPGACICRGNGYC;
[SEQ ID NO: 3]
CPRILKKCRRDSDCPGACICRGNGYC;
[SEQ ID NO: 4]
CPKILQRCRRDSDCPGACICLGNGYC;
[SEQ ID NO: 5]
CPKILKKCRHDSDCPGACICRGNGYC;
[SEQ ID NO: 6]
CFRILKKCRRDSDCPGACICRGNGYC;
or
[SEQ ID NO: 7]
CFRIWKKCRRDSDCPGACICRGNGYC.

2. A proteinaceous molecule comprising an amino acid sequence represented by Formula I:

CX₁X₂X₃X₄X₅X₆CX₇X₈DSDCPGACICX₉X₁₀X₁₁X₁₂X₁₃C (I)

wherein:
X₁ is selected from P and modified forms thereof; C and modified forms thereof; and F and modified forms thereof;
X₂ is selected from basic amino acid residues including K, R, H and modified forms thereof; and small amino acid residues including S, T, A, G and modified forms thereof;
X₃ is selected from small amino acid residues including S, T, A, G and modified forms thereof; aromatic amino acid residues including F, Y, W, 4-F-Phe, 4-Me-Phe and modified forms thereof; and hydrophobic amino acid residues including V, L, I, Ne and modified forms thereof;
X₄ is selected from small amino acid residues including S, T, A, G and modified forms thereof; aromatic amino acid residues including F, Y, W and modified forms thereof; hydrophobic amino acid residues including V, L, I, Ne and modified forms thereof; and acidic amino acid residues including D, E, hGlu and modified forms thereof;
X₅ is selected from small amino acid residues including S, T, A, G and modified forms thereof; aromatic amino acid residues including F, Y, W and modified forms thereof; hydrophobic amino acid residues including V, L, I, M, Ne and modified forms thereof; basic amino acid residues including K, R, H and modified forms thereof; and amide containing amino acid residues including N, Q and modified forms thereof;
X₆ is selected from any amino acid residue;
X₇ is selected from basic amino acid residues including K, R, H and modified forms thereof; amide containing amino acid residues including N, Q and modified forms thereof; small amino acid residues including S, T, A, G and modified forms thereof; acidic amino acid residues including D, E and modified forms thereof; and hydrophobic amino acid residues including V, L, I, Ne and modified forms thereof;
X₈ is selected from basic amino acid residues including K, R, H and modified forms thereof; and amide containing amino acid residues including N, Q and modified forms thereof;
X₉ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof;
X₁₀ is selected from P and modified forms thereof; small amino acid residues including S, T, A, G and modified forms thereof; aromatic amino acid residues including F, Y, W and modified forms thereof; and basic amino acid residues including K, R, H and modified forms thereof;
X₁₁ is selected from amide containing amino acid residues including N, Q and modified forms thereof; small amino acid residues including S, T, A, G and modified forms thereof; and basic amino acid residues including K, R, H and modified forms thereof;
X₁₂ is selected from small amino acid residues including S, T, A, G and modified forms thereof; basic amino acid residues including K, R, H and modified forms thereof; and amide containing amino acid residues including N, Q and modified forms thereof; and
X₁₃ is selected from basic amino acid residues including K, R, H and modified forms thereof; aromatic amino acid residues including F, Y, W and modified forms thereof; and hydrophobic amino acid residues including V, L, I, Ne and modified forms thereof; wherein the proteinaceous molecule is other than a proteinaceous molecule comprising or consisting of the amino acid sequence of any one of SEQ ID NOs: 1 to 7:

[SEQ ID NO: 1]
CPKILKKCRRDSDCPGACICRGNGYC;
[SEQ ID NO: 2]
CPKILQRCRRDSDCPGACICRGNGYC;
[SEQ ID NO: 3]
CPRILKKCRRDSDCPGACICRGNGYC;
[SEQ ID NO: 4]
CPKILQRCRRDSDCPGACICLGNGYC;
[SEQ ID NO: 5]
CPKILKKCRHDSDCPGACICRGNGYC;
[SEQ ID NO: 6]
CFRILKKCRRDSDCPGACICRGNGYC;
or
[SEQ ID NO: 7]
CFRIWKKCRRDSDCPGACICRGNGYC.

3. The proteinaceous molecule according to embodiment 1 or embodiment 2, wherein X₁ is P or C.

4. The proteinaceous molecule according to any one of embodiments 1-3, wherein X₂ is R, G or K.

5. The proteinaceous molecule according to any one of embodiments 1-4, wherein X₂ is R.

6. The proteinaceous molecule according to any one of embodiments 1-5, wherein X₃ is I, L, V, F, G, Nle, 4-F-Phe or 4-Me-Phe.

7. The proteinaceous molecule according to any one of embodiments 1-6, wherein X₄ is G, L, E, Y, V, W or Ne.

8. The proteinaceous molecule according to any one of embodiments 1-7, wherein X₅ is selected from aromatic amino acid residues including F, Y, W and modified forms thereof; hydrophobic amino acid residues including V, L, I, Ne and modified forms thereof; and basic amino acid residues including K, R, H and modified forms thereof.

9. The proteinaceous molecule according to any one of embodiments 1-8, wherein X₅ is R, K, V, W or L.

10. The proteinaceous molecule according to any one of embodiments 1-9, wherein X₆ is selected from small amino acid residues including S, T, A, G and modified forms thereof; aromatic amino acid residues including F, Y, W and modified forms thereof; hydrophobic amino acid residues including V, L, I, Ne and modified forms thereof; and basic amino acid residues including K, R, H and modified forms thereof.

11. The proteinaceous molecule according to any one of embodiments 1-10, wherein X₆ is K, L, Y, W, R, A or V.

12. The proteinaceous molecule according to any one of embodiments 1-11, wherein X₇ is K or R.

13. The proteinaceous molecule according to any one of embodiments 1-12, wherein X₈ is K or R.

14. The proteinaceous molecule according to any one of embodiments 1-13, wherein X₉ is R, I, A, Y or V.

15. The proteinaceous molecule according to any one of embodiments 1-14, wherein X₁₀ is G, A, R, P or F.

16. The proteinaceous molecule according to any one of embodiments 1-15, wherein X₁₁ is N, T, R, G or K.

17. The proteinaceous molecule according to any one of embodiments 1-16, wherein X₁₂ is G, R, T or K.

18. The proteinaceous molecule according to any one of embodiments 1-17, wherein X₁₃ is Y, F, L, W or H.

19. The proteinaceous molecule according to any one of embodiments 1-18, wherein:
X₁ is P;
X₂ is R;
X₃ is I, F, L, V or 4-F-Phe;
X₄ is L, E, Nle, V, W or G;
X₅ is K, R, V or W;
X₆ is K, L, Y, W, R or A;
X₇ is R or K;
X₈ is R or K;
X₉ is R, I, A or Y;
X₁₀ is G, A, R or P;
X₁₁ is N, T, R or G;
X₁₂ is R, T, G or K; and
X₁₃ is Y, F, L or W.

20. The proteinaceous molecule according to embodiment 19, wherein:
X₁ is P;
X₂ is R;
X₃ is I, F, or 4-F-Phe;
X₄ is L, E, Nle, V, W or G;
X₅ is K or R;
X₆ is K, L or A;
X₇ is R or K;
X₈ is R or K;
X₉ is R;
X₁₀ is G or A;
X₁₁ is N or T;
X₁₂ is R or G; and
X₃ is Y or F.

21. The proteinaceous molecule according to any one of embodiments 1-20, wherein the proteinaceous molecule comprises, consists or consists essentially of an amino acid sequence represented by any one of SEQ ID NOs: 8-36:

[SEQ ID NO: 8]
DGGICPRIGRLCRRDSDCPGACICRATRFCGSGY;
[SEQ ID NO: 9]
GGICPRIGRLCRRDSDCPGACICRATRFCGSGYD;
[SEQ ID NO: 10]
GGICPRIGRLCRRDSDCPGACICRATRFCGSGSD;
[SEQ ID NO: 11]
DGGICPRILVYCRRDSDCPGACICIRRTYCGSGS;
[SEQ ID NO: 12]
GGICPRILVYCRRDSDCPGACICIRRTYCGSGSD;
[SEQ ID NO: 13]
DGGRCPRLLRWCRRDSDCPGACICARGGLCGSGS;
[SEQ ID NO: 14]
GGRCPRLLRWCRRDSDCPGACICARGGLCGSGSD;
[SEQ ID NO: 15]
DGGVCPRVGWRCRRDSDCPGACICYPTKWCGSGS;
[SEQ ID NO: 16]
GGVCPRVGWRCRRDSDCPGACICYPTKWCGSGSD;
[SEQ ID NO: 17]
DGGRCCGGYLVCRRDSDCPGACICVFKKHCGSGS;
[SEQ ID NO: 18]
GGRCCGGYLVCRRDSDCPGACICVFKKHCGSGSD;
[SEQ ID NO: 19]
DGGICPRIGRLCRRDSDCPGACICRGNGYCGSGS;
[SEQ ID NO: 20]
GGICPRIGRLCRRDSDCPGACICRGNGYCGSGSD;
[SEQ ID NO: 21]
DGGVCPKILKKCRRDSDCPGACICRATRFCGSGS;
[SEQ ID NO: 22]
GGVCPKILKKCRRDSDCPGACICRATRFCGSGSD;
[SEQ ID NO: 23]
RICPRIGRLCRRDSDCPGACICRATRFCG;
[SEQ ID NO: 24]
GGICPRIGRLCKRDSDCPGACICRATRFCGSGSD;
[SEQ ID NO: 25]
GGICPRIGRLCRKDSDCPGACICRATRFCGSGSD;
[SEQ ID NO: 26]
GGICPRIGRLCRRDSDCPGACICRATRFCGSGKD;
[SEQ ID NO: 27]
GGICPRFGRLCRRDSDCPGACICRATRFCGSGSD;
[SEQ ID NO: 28]
GGRCPRIGRLCRRDSDCPGACICRATRFCGSGSD;
[SEQ ID NO: 29]
RVCPR[4-F-Phe]EKKCRRDSDCPGACICRGNGYCG;
[SEQ ID NO: 30]
RVCPR[4-F-Phe]VKKCRRDSDCPGACICRGNGYCG;
[SEQ ID NO: 31]
RVCPR[4-F-Phe]WKKCRRDSDCPGACICRGNGYCG;
[SEQ ID NO: 32]
RVCPR [4-F-Phe]ERKCRRDSDCPGACICRGNGYCG;
[SEQ ID NO: 33]
RVCPR [4-F-Phe]VRKCRRDSDCPGACICRGNGYCG;
[SEQ ID NO: 34]
RVCPR[4-F-Phe]WRKCRRDSDCPGACICRGNGYCG;
[SEQ ID NO: 35]
RVCPR[4-F-Phe][Nle]KACRRDSDCPGACICRGNGYCG;
or
[SEQ ID NO: 36]
GGVCPR[4-F-Phe ]EKKCRRDSDCPGACICRGNGYCGSGSD.

22. The proteinaceous molecule according to embodiment 21, wherein the proteinaceous molecule comprises, consists or consists essentially of an amino acid sequence represented by SEQ ID NO: 8 or 19.

23. A proteinaceous molecule comprising, consisting or consisting essentially of an amino acid sequence represented by Formula VII:

CPRIGRX₆CX₇X₈X₂₇X₂₈X₂₉CX₂₃GACX₂₆CRX₁₀TX₁₂FC (VII)

wherein:
X₆ is selected from L, V, T, I and modified forms of any of the foregoing amino acids;
X₇ is selected from R, W, V, T, S, Q, N, M, Nle, L, K, I, F, E, D, A and modified forms of any of the foregoing amino acids;
X₈ is selected from R, Y, V, T, Q, M, Nle, L, K, I, H, F, E, A and modified forms of any of the foregoing amino acids;
X₂₇ is selected from D, T, N, H and modified forms of any of the foregoing amino acids;
X₂₈ is selected from S, T, A and modified forms of any of the foregoing amino acids;
X₂₉ is selected from D, E and modified forms of any of the foregoing amino acids;
X₂₃ is selected from P, Y, M, Nle, L, I, F and modified forms of any of the foregoing amino acids;
X₂₆ is selected from I, V, K and modified forms of any of the foregoing amino acids;
X₁₀ is selected from A, V, T, S, R, P, K and modified forms of any of the foregoing amino acids; and
X₁₂ is selected from R, K, H, G and modified forms of any of the foregoing amino acids.

24. The proteinaceous molecule according to any one of embodiments 1-23, wherein the proteinaceous molecule is a cyclic molecule.

25. The proteinaceous molecule according to embodiment 24, wherein the proteinaceous molecule is cyclized through N-to-C cyclization.

26. The proteinaceous molecule according to any one of embodiments 1-25, wherein the six cysteine residues in the proteinaceous molecule are bonded in pairs to form three disulfide bonds.

27. The proteinaceous molecule according to embodiment 26, wherein the disulfide bonds are formed between the side chains of Cys 1 and Cys 18, Cys 8 and Cys 20, and Cys 14 and Cys 26 (numbered in accordance with Formula I).

28. A composition comprising, consisting or consisting essentially of a proteinaceous molecule according to any one of embodiments 1-27 and a pharmaceutically acceptable carrier or diluent.

29. A method of treating or inhibiting the development of a condition in which inhibiting FXIIa activity is associated with effective treatment or inhibition, comprising administering the proteinaceous molecule according to any one of embodiments 1-27.

30. The method according to embodiment 29, wherein the condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, sickle cell disease, thrombophilia, and thrombosis resulting from a medical implant, device or extracorporeal circulation procedure in which blood is exposed to an artificial surface that promotes thrombosis.

31. The method according to embodiment 29, wherein the condition is an inflammatory condition.

32. The method according to embodiment 31, wherein the condition is hereditary angioedema, anaphylaxis, rheumatoid arthritis, pancreatitis, sepsis, multiple sclerosis or lupus.

33. A method of inhibiting an activity of FXIIa, comprising contacting FXIIa with a proteinaceous molecule according to any one of embodiments 1-27.

34. A method of treating or inhibiting the development of a thrombosis in a subject, comprising administering a proteinaceous molecule according to any one of embodiments 1-27 to the subject.

35. A method of inhibiting coagulation in a subject, comprising administering a proteinaceous molecule according to any one of embodiments 1-27 to the subject.

36. A method for inhibiting thrombus or embolus formation in a subject, comprising administering the proteinaceous molecule according to any one of embodiments 1-27 to the subject to thereby inhibit thrombus or embolus formation in the subject.

37. A method for treating or inhibiting the development of a thromboembolism-associated condition in a subject, comprising administering the proteinaceous molecule according to any one of embodiments 1-27 to the subject.

38. The method according to embodiment 37, wherein the thromboembolism-associated condition is selected from an arterial cardiovascular thromboembolic disorder, a venous cardiovascular or cerebrovascular thromboembolic disorder and a thromboembolic disorder in a chamber of the heart or in the peripheral circulation.

39. The method according to embodiment 37, wherein the thromboembolism-associated condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from a medical implant, device or extracorporeal circulation (ECMO, cardiopulmonary bypass) procedure in which blood is exposed to an artificial surface that promotes thrombosis.

40. The method according to embodiment 39, wherein the medical implant or device is selected from a prosthetic valve, artificial valve, indwelling catheter, stent, blood oxygenator, shunt, vascular access port, ventricular assist device and artificial heart or heart chamber, and vessel graft.

41. The method according to embodiment 39, wherein the procedure is selected from cardiopulmonary bypass, percutaneous coronary intervention and hemodialysis.

42. The method according to embodiment 37, wherein the thromboembolism-associated condition is selected from acute coronary syndrome, stroke, deep vein thrombosis and pulmonary embolism.

43. A method for treating or inhibiting the development of a thrombosis-associated hematologic disorder in a subject, comprising administering the proteinaceous molecule according to any one of embodiments 1-27 to the subject.

44. The method according to embodiment 43, wherein the hematologic disorder is sickle cell disease or thrombophilia.

45. An in vitro method for identifying a disulfide rich peptide which binds to a target substance comprising:
a) preparing an mRNA library based on a disulfide rich peptide scaffold;
b) ligating mRNA in the library to puromycin to form mRNA-puromycin conjugates;
c) translating the mRNA-puromycin conjugates using a prokaryotic translation system to produce mRNA-puromycin-peptide conjugates;
d) reverse transcribing the conjugates to form mRNA:cDNA-puromycin-peptide conjugates;
e) performing affinity selection against the target substance to select for mRNA:cDNA-puromycin-peptide conjugates that bind to the target substance;
f) performing nucleic acid amplification on the cDNA of the selected mRNA:cDNA-puromycin-peptide conjugates to generate an enriched cDNA library; and
g) sequencing the enriched cDNA library to identify a disulfide rich peptide which binds to the target substance.

46. The method according to embodiment 45, wherein the disulfide rich peptide contains at least six cysteine residues.

47. The method according to embodiment 45 or embodiment 46, wherein the disulfide rich peptide contains at least three disulfide bonds.

48. The method according to any one of embodiments 45-47, wherein the disulfide rich peptide contains a cystine knot motif.

49. The method according to any one of embodiments 45-48, wherein the disulfide rich peptide has at least about 2-fold greater binding affinity for the target substance than the disulfide rich peptide scaffold.

50. The method according to any one of embodiments 45-49, wherein the disulfide rich peptide has at least about 2-fold greater selectivity for the target substance than the disulfide rich peptide scaffold.

51. The method according to any one of embodiments 45-50, wherein the prokaryote is E. coli.

52. The method according to embodiment 51, wherein the prokaryotic translation system does not comprise release factor 1 (RF1).

53. The method according to any one of embodiments 45-52, wherein the prokaryotic translation system comprises tRNAs, initiation factors, elongation factors, release factors, T7 RNA polymerase, nucleoside triphosphates, aminoacyl-tRNA synthetases (ARS), ribosomes and the 20 natural amino acids.

54. The method according to embodiment 53, wherein the tRNAs, initiation factors, elongation factors and/or release factors are from E. coli.

55. The method according to any one of embodiments 45-54, wherein the translation system comprises E. coli ribosome, initiation factor 1 (IF1), initiation factor 2 (IF2), initiation factor 3 (IF3), elongation factor G (EF-G), elongation factor thermo unstable (EF-Tu), elongation factor thermo stable (EF-Ts), release factor 2 (RF2), release factor 3 (RF3), ribosome release factor (RRF), alanyl-tRNA synthetase (AlaRS), arginyl-tRNA synthetase (ArgRS), asparaginyl-tRNA synthetase (AsnRS), aspartyl-tRNA synthetase (AspRS), cysteinyl-tRNA synthetase (CysRS), glutamyl-tRNA synthetase (GluRS), glutaminyl-tRNA synthetase (GInRS), glycyl-tRNA synthetase (GlyRS), histidyl-tRNA synthetase (HisRS), isoleucyl-tRNA synthetase (IleRS), leucyl-tRNA synthetase (LeuRS), lysyl-tRNA synthetase (LysRS), methionyl-tRNA synthetase (MetRS), phenylalanyl-tRNA synthetase (PheRS), prolyl-tRNA synthetase (ProRS), seryl-tRNA synthetase (SerRS), threonyl-tRNA synthetase (ThrRS), tryptophanyl-tRNA synthetase (TrpRS), tyrosyl-tRNA synthetase (TyrRS), valyl-tRNA synthetase (VaIRS), methionyl-tRNA formyltransferase (MTF), T7 RNA polymerase, E. coli total tRNA, adenosine triphosphate (ATP), guanosine triphosphate (GTP), cytidine triphosphate (CTP) and uridine triphosphate (UTP) and the 20 natural amino acids.

56. The method according to embodiment 55, wherein the translation system further comprises inorganic pyrophosphatase, nucleoside diphosphate kinase, creatine phosphate, 10-formyl-5,6,7,8-tetrahydrofolic acid, spermidine, dithiothreitol (DTT), potassium acetate, magnesium acetate, HEPES-KOH buffer, myokinase and creatine kinase.

57. The method according to any one of embodiments 45-56, wherein, prior to step g), an mRNA library is prepared based on the enriched cDNA library produced in step f), and steps b) to f) are repeated.

58. A proteinaceous molecule according to any one of embodiments 1-27 for use in therapy.

59. A proteinaceous molecule according to any one of embodiments 1-27 for use in treating or inhibiting the development of a condition in which inhibiting FXIIa activity is associated with effective treatment or inhibition.

60. The proteinaceous molecule for use according to embodiment 59, wherein the condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, sickle cell disease, thrombophilia, and thrombosis resulting from a medical implant, device or extracorporeal circulation procedure in which blood is exposed to an artificial surface that promotes thrombosis.

61. The proteinaceous molecule for use according to embodiment 59, wherein the condition is an inflammatory condition.

62. The proteinaceous molecule for use according to embodiment 61, wherein the condition is hereditary angioedema, anaphylaxis, rheumatoid arthritis, pancreatitis, sepsis, multiple sclerosis or lupus.

63. A proteinaceous molecule according to any one of embodiments 1-27 for use in inhibiting an activity of FXIIa.

64. A proteinaceous molecule according to any one of embodiments 1-27 for use in treating or inhibiting the development of a thrombosis in a subject.

65. A proteinaceous molecule according to any one of embodiments 1-27 for use in inhibiting coagulation in a subject.

66. A proteinaceous molecule according to any one of embodiments 1-27 for use in inhibiting thrombus or embolus formation in a subject.

67. A proteinaceous molecule according to any one of embodiments 1-27 for use in treating or inhibiting the development of a thromboembolism-associated condition in a subject.

68. The proteinaceous molecule for use according to embodiment 67, wherein the thromboembolism-associated condition is selected from an arterial cardiovascular thromboembolic disorder, a venous cardiovascular or cerebrovascular thromboembolic disorder and a thromboembolic disorder in a chamber of the heart or in the peripheral circulation.

69. The proteinaceous molecule for use according to embodiment 67, wherein the thromboembolism-associated condition is selected from unstable angina or other abdominal aortic aneurysm, acute coronary syndrome, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from a medical implant, device or extracorporeal circulation (ECMO, cardiopulmonary bypass) procedure in which blood is exposed to an artificial surface that promotes thrombosis.

70. The proteinaceous molecule for use according to embodiment 69, wherein the medical implant or device is selected from a prosthetic valve, artificial valve, indwelling catheter, stent, blood oxygenator, shunt, vascular access port, ventricular assist device and artificial heart or heart chamber, and vessel graft.

71. The proteinaceous molecule for use according to embodiment 69, wherein the procedure is selected from cardiopulmonary bypass, percutaneous coronary intervention and hemodialysis.

72. The proteinaceous molecule for use according to embodiment 67, wherein the thromboembolism-associated condition is selected from acute coronary syndrome, stroke, deep vein thrombosis and pulmonary embolism.

73. A proteinaceous molecule according to any one of embodiments 1-27 for use in treating or inhibiting the development of a thrombosis-associated hematologic disorder in a subject.

74. The proteinaceous molecule for use according to embodiment 73, wherein the hematologic disorder is sickle cell disease or thrombophilia.

1.-28. (canceled)

29. An in vitro method for identifying a disulfide rich peptide which binds to a target substance comprising:
a) preparing an mRNA library based on a disulfide rich peptide scaffold;
b) ligating mRNA in the library to puromycin to form mRNA-puromycin conjugates;
c) translating the mRNA-puromycin conjugates using a prokaryotic translation system to produce mRNA-puromycin-peptide conjugates;
d) reverse transcribing the conjugates to form mRNA:cDNA-puromycin-peptide conjugates;
e) performing affinity selection against the target substance to select for mRNA:cDNA-puromycin-peptide conjugates that bind to the target substance;
f) performing nucleic acid amplification on the cDNA of the selected mRNA:cDNA-puromycin-peptide conjugates to generate an enriched cDNA library; and
g) sequencing the enriched cDNA library to identify a disulfide rich peptide which binds to the target substance.

30. The method of claim 29, wherein the disulfide rich peptide contains at least six cysteine residues.

31. The method of claim 30, wherein the disulfide rich peptide contains at least three disulfide bonds.

32. The method of claim 31, wherein the disulfide rich peptide contains a cystine knot motif.

33. The method of claim 29, wherein the disulfide rich peptide has at least about 2-fold greater binding affinity for the target substance than the disulfide rich peptide scaffold.

34. The method of claim 33, wherein the disulfide rich peptide has at least about 2-fold greater selectivity for the target substance than the disulfide rich peptide scaffold.

35. The method of claim 29, wherein the prokaryote is Escherichia coli.

36. The method of claim 35, wherein the prokaryotic translation system does not comprise release factor 1 (RF1).

37. The method of claim 36, wherein the prokaryotic translation system comprises tRNAs, initiation factors, elongation factors, release factors, T7 RNA polymerase, nucleoside triphosphates, aminoacyl-tRNA synthetases (ARS), ribosomes and the 20 natural amino acids.

38. The method of claim 37, wherein the prokaryotic translation system comprises E. coli ribosome, initiation factor 1 (IF1), initiation factor 2 (IF2), initiation factor 3 (IF3), elongation factor G (EF-G), elongation factor thermo unstable (EF-Tu), elongation factor thermo stable (EF-Ts), release factor 2 (RF2), release factor 3 (RF3), ribosome release factor (RRF), alanyl-tRNA synthetase (AlaRS), arginyl-tRNA synthetase (ArgRS), asparaginyl-tRNA synthetase (AsnRS), aspartyl-tRNA synthetase (AspRS), cysteinyl-tRNA synthetase (CysRS), glutamyl-tRNA synthetase (GluRS), glutaminyl-tRNA synthetase (GlnRS), glycyl-tRNA synthetase (GlyRS), histidyl-tRNA synthetase (HisRS), isoleucyl-tRNA synthetase (IleRS), leucyl-tRNA synthetase (LeuRS), lysyl-tRNA synthetase (LysRS), methionyl-tRNA synthetase (MetRS), phenylalanyl-tRNA synthetase (PheRS), prolyl-tRNA synthetase (ProRS), seryl-tRNA synthetase (SerRS), threonyl-tRNA synthetase (ThrRS), tryptophanyl-tRNA synthetase (TrpRS), tyrosyl-tRNA synthetase (TyrRS), valyl-tRNA synthetase (ValRS), methionyl-tRNA formyltransferase (MTF), T7 RNA polymerase, E. coli total tRNA, adenosine triphosphate (ATP), guanosine triphosphate (GTP), cytidine triphosphate (CTP) and uridine triphosphate (UTP) and the 20 natural amino acids.

39. The method of claim 38, wherein the translation system further comprises inorganic pyrophosphatase, nucleoside diphosphate kinase, creatine phosphate, 10-formyl-5,6,7,8-tetrahydrofolic acid, spermidine, dithiothreitol (DTT), potassium acetate, magnesium acetate, HEPES-KOH buffer, myokinase and creatine kinase.

40. The method of claim 29, wherein prior to step g), an mRNA library is prepared based on the enriched cDNA library produced in step f), and steps b) to f) are repeated.

41. The method of claim 32, wherein the disulfide rich peptide scaffold is a cyclotide.

42. The method of claim 41, wherein the disulfide rich peptide scaffold is a peptide comprising the amino acid sequence of SEQ ID NO: 1, 43 or 44.
